(19) United States
(12) Patent Application Publication
(10) Pub. No.: US 2025/0051410 A1
(43) Pub. Date: Feb. 13, 2025

(54) LILRB POLYPEPTIDES AND USES THEREOF

(71) Applicant: KAHR Medical Ltd., ModiIn Makabim-ReUt (IL)

(72) Inventors: Ami TAMIR, Rehovot (IL); Itai BLOCH, Rehovot (IL)

(73) Assignee: KAHR Medical Ltd., ModiIn Makabim-ReUt (IL)

(21) Appl. No.: 18/722,601

(22) PCT Filed: Dec. 22, 2022
(86) PCT No.: PCT/IL2022/051378

§ 371 Date: Jun. 21, 2024

Related U.S. Application Data
(60) Provisional application No. 63/407,763, filed on Sep. 19, 2022.
(60) Provisional application No. 63/293,264, filed on Dec. 23, 2021.

(51) Int. Cl. C07K 14/47; A61K 38/00; A61P 37/04; C07K 14/705

(52) U.S. Cl. CPC C07K 14/4705; A61P 37/04; C07K 14/70503; A61K 38/00; C07K 2319/30

(57) ABSTRACT

LILRB polypeptides are provided. Accordingly, there is provided a LILRB polypeptide capable of binding an HLA-G polypeptide as set forth in SEQ ID NO: 3 and having at least one mutation located within amino acid positions 40-60 of a D1 domain of LILRB, wherein the LILRB polypeptide has an increased stability and/or increased affinity to HLA-G compared to a LILRB polypeptide of the same length and sequence not comprising the at least one mutation. Also provided are polynucleotides encoding the LILRB polypeptide, host cells expressing the LILRB polypeptide and methods of producing and using same.

### RELATED APPLICATION/S

This application claims the benefit of priority of U.S. Provisional Patent Application No. 63/293,264 filed on Dec. 23, 2021 and U.S. Provisional Patent Application No. 63/407,763 filed on Sep. 19, 2022, the contents of which are incorporated herein by reference in their entirety.

### SEQUENCE LISTING STATEMENT

The XML file, entitled 94087.xml, created on 21 Dec. 2022, comprising 206,301 bytes, submitted concurrently with the filing of this application is incorporated herein by reference.

### FIELD AND BACKGROUND OF THE INVENTION

The present invention, in some embodiments thereof, relates to LILRB polypeptides and uses thereof.

One of the major immune escape mechanisms in cancer is the expression of inhibitory molecules on the cancerous cell' surface impairing immune activation signaling. Many of these inhibitory molecules are considered as Immune Check Points (ICP) belonging to numerous inhibitory pathways first demonstrated to maintain self-tolerance and to modulate the duration and amplitude of physiological immune responses within peripheral tissues to avoid collateral tissue damages.

HLA-G, a non-classical MHC class I molecule, is a molecule that was first known to confer protection to the fetus from destruction by the immune system of its mother, thus critically contributing to fetal-maternal tolerance [Carosella et al. Adv Immunol (2015) 127:33-144]. HLA-G, membrane-bound or soluble, strongly binds its inhibitory receptors on immune cells, inhibits the functions of these effectors, and so functions as an ICP molecule to induce immune inhibition. The reported receptors for HLA-G include leukocyte immunoglobulin-like receptor B1 (LILRB1, also known as ILT2 or CD85j), LILRB2 (also known as ILT4 or CD85d), as well as KIR2DL4 (killer cell Ig-like receptor 2DL4, also known as CD158d) expressed on natural killer cells and some T cells [e.g. Shiroishi M et al., Proc Natl Acad Sci USA. (2006) 103 (44): 16412-16417; Attia J V D, et al. Int J Mol Sci. (2020) 21 (22): 8678]. LILRB1 is expressed on human macrophages, some T cells, NK cells, B cells, monocytes, various dendritic cell subsets including myeloid, plasmacytoid and tolerogenic DCs; while LILRB2 is expressed on human monocytes, B cells and at lower levels on the cell surface of myeloid and plasmacytoid dendritic cells (Katz H R. Adv Immunol. (2006) 91:251-272; Kang X. et al. Cell Cycle. (2016) 15 (1): 25-40). Both LILRB1 and LILRB2 have immune-receptor tyrosine-based inhibitory motifs in their cytoplasmic tails to recruit the protein tyrosine phosphatase SHP-1, resulting in the inhibitory signaling. Because the LILRBs are expressed on a wide range of leukocytes and mediate inhibitory signals, HLA-G is believed to have a pivotal role in a broad range of immune suppression functions in the placenta

While HLA-G is predominantly expressed on placenta trophoblasts and thymic epithelial cells (e.g. Shiroishi M et al., 2006), it has been shown that many tumors (including e.g. pancreatic, breast, skin, colorectal, gastric, ovarian) upregulate expression of HLA-G (e.g. Lin, A. et al, Mol Med. 21 (2015) 782-791; Amiot, L., et al, Cell Mol Life Sci. 68 (2011) 417-431). Moreover, it has been shown that tumor cells escape host immune surveillance by inducing immune tolerance/suppression via HLA-G expression; and that expression of HLA-G is associated with poor prognosis. Furthermore, HLA-G can also be neo-expressed and/or up-regulated in other pathological conditions such as viral infections, auto-immune and inflammatory diseases or after allo-transplantation. For instance, viruses such as HCMV, HSV-1, RABV, HCV, IAV and HIV-1 seem to up-regulate the expression of HLA-G to prevent infected cells from being recognized and attacked by immune cells.

As the relevance of the HLA-G-LILRB1/2 signaling as an escape mechanism employed by pathologic cells has been widely demonstrated, several approaches targeting this pathway have been developed [e.g. Carosella E D et al., Trends Immunol (2008) 29:125-32; Carosella E D et al., Blood (2008) 111:4862-70; Yan W H, Endocr Metab Immune Disord Drug Targets (2011) 11:76-89; Blaschitz A et al., Hum Immunol 2000; 61:1074-85; Menier C, et al., Hum Immunol 2003; 64:315-26; François et al., J Immunother Cancer (2021) 9 (3): e001998; US Patent Application Publication No. US20210301020; and International patent application Nos. WO2014/072534, WO2017207775 and WO2018091580.

### Additional Background Art Includes

Shiroishi M et al., Proc Natl Acad Sci USA. (2003) 100 (15): 8856-61;
Shiroishi M et al., J Biol Chem. (2006) 281 (15): 10439-47;
Clements C S et al., Proc Natl Acad Sci USA. (2005) 102 (9): 3360-5.

### SUMMARY OF THE INVENTION

According to an aspect of some embodiments of the present invention there is provided a LILRB polypeptide capable of binding an HLA-G polypeptide as set forth in SEQ ID NO: 3 and having at least one mutation located within amino acid positions 40-60 of a D1 domain of LILRB, wherein the LILRB polypeptide has an increased stability and/or increased affinity to the HLA-G compared to a LILRB polypeptide of the same length and sequence not comprising the at least one mutation.

According to some embodiments of the invention, the LILRB is selected from the group consisting of LILRB1 and LILRB2.

According to some embodiments of the invention, the LILRB is LILRB2 and the at least one mutation is at an amino acid position selected from the group consisting of S45, I49, T50 and V57 corresponding to SEQ ID NO: 1.

According to an aspect of some embodiments of the present invention there is provided a LIL2B2 polypeptide capable of binding an HLA-G polypeptide as set forth in SEQ ID NO: 3 and having at least one mutation at an amino acid position selected from the group consisting of S45, 149, T50 and V57 corresponding to SEQ ID NO: 1, wherein the LILRB2 polypeptide has an increased stability and/or increased affinity to the HLA-G compared to a LILRB2 polypeptide of the same length and sequence not comprising the at least one mutation.

According to some embodiments of the invention, the mutation in S45 comprises a S45R, S45N, S45Q, S45H, S45L, S45K, S45M, S45F, S45W or S45Y mutation, the mutation in I49 comprises a I49R, I49K, I49F or I49Y mutation, the mutation in T50 comprises a T50R, T50N, T50L, T50K, T50F, T50W or T50Y mutation, and/or the mutation in V57 comprises a V57R, V57K, V57F or V57W mutation.

According to some embodiments of the invention, the mutation in S45 comprises a S45Q mutation, the mutation in I49 comprises a I49K mutation, the mutation in T50 comprises a T50F mutation, and/or the mutation in V57 comprises a V57R mutation.

According to some embodiments of the invention, the at least one mutation comprises at least two mutations.

According to some embodiments of the invention, the at least one mutation comprises a mutation at the S45 and an additional mutation at the I49, T50 and/or V57.

According to some embodiments of the invention, the LILRB2 polypeptide comprising S45N and T50R mutations, S45Y and T50K mutations, S45R and I49F mutations, S45Q and V57R mutations, S45Q and I49K mutations, or S45Y and T50N mutations.

According to some embodiments of the invention, the LILRB2 polypeptide having increased affinity to the HLA-G compared to a LILRB2 polypeptide as set forth in SEQ ID NO: 1.

According to some embodiments of the invention, the LILRB2 polypeptide having increased stability compared to a LILRB2 polypeptide as set forth in SEQ ID NO: 1.

According to some embodiments of the invention, the LILRB2 polypeptide amino acid sequence is at least 90% identical to an amino acid sequence selected from the group consisting of SEQ ID NO: 5, 7, 9, 11, 13, 15, 17, 19, 21 and 23.

According to some embodiments of the invention, the LILRB2 polypeptide amino acid sequence comprises an amino acid sequence selected from the group consisting of SEQ ID NO: 5, 7, 9, 11, 13, 15, 17, 19, 21 and 23.

According to some embodiments of the invention, the LILRB2 polypeptide amino acid sequence is as set forth in SEQ ID NO: 5, 7, 9, 11, 13, 15, 17, 19, 21 or 23.

According to some embodiments of the invention, the LILRB is LILRB1 and the at least one mutation is at an amino acid position selected from the group consisting of T43, 147, T48 and V55 corresponding to SEQ ID NO: 102.

According to some embodiments of the invention, the mutation in T43 comprises a T43R, T43N, T43Q, T43H, T43L, T43K, T43M, T43F, T43W or T43Y mutation, the mutation in 147 comprises a 147R, 147K, 147F or 147Y mutation, the mutation in T48 comprises a T48R, T48N, T48L, T48K, T48F, T48W or T48Y mutation, and/or the mutation in V55 comprises a V55R, V55K, V55F or V55W mutation.

According to some embodiments of the invention, the mutation in V55 comprises a V55R mutation.

According to some embodiments of the invention, the LILRB1 polypeptide having increased affinity to the HLA-G compared to a LILRB1 polypeptide as set forth in SEQ ID NO: 102.

According to some embodiments of the invention, the LILRB1 polypeptide having increased stability compared to a LILRB1 polypeptide as set forth in SEQ ID NO: 102.

According to an aspect of some embodiments of the present invention there is provided a composition of matter comprising the LILRB polypeptide and a non-proteinaceous moiety attached to the LILRB polypeptide.

According to an aspect of some embodiments of the present invention there is provided a composition of matter comprising the LILRB2 polypeptide and a non-proteinaceous moiety attached to the LILRB2 polypeptide.

According to some embodiments of the invention, the non-proteinaceous moiety is selected from the group consisting of a drug, a chemical, a small molecule, a polynucleotide, a detectable moiety, polyethylene glycol (PEG), Polyvinyl pyrrolidone (PVP), poly (styrene comaleic anhydride) (SMA), and divinyl ether and maleic anhydride copolymer (DIVEMA).

According to some embodiments of the invention, the non-proteinaceous moiety is a dimerizing moiety.

According to an aspect of some embodiments of the present invention there is provided a fusion polypeptide comprising the LILRB polypeptide attached to a heterologous proteinaceous moiety.

According to an aspect of some embodiments of the present invention there is provided a fusion polypeptide comprising the LILRB2 polypeptide attached to a heterologous proteinaceous moiety.

According to some embodiments of the invention, the heterologous proteinaceous moiety is a dimerizing moiety.

According to some embodiments of the invention, the heterologous proteinaceous moiety comprises an Fc domain of an antibody or a fragment thereof.

According to some embodiments of the invention, the Fc domain is of IgG1 or IgG4. According to some embodiments of the invention, the Fc domain is modified to alter it's binding to an Fc receptor, reduce an immune activating function thereof and/or improve half-life of the fusion.

According to an aspect of some embodiments of the present invention there is provided a dimer comprising the LILRB polypeptide, the composition or the fusion polypeptide.

According to an aspect of some embodiments of the present invention there is provided a dimer comprising the LILRB2 polypeptide, the composition or the fusion polypeptide.

According to some embodiments of the invention, the dimer being a heterodimer.

According to some embodiments of the invention, a first monomer of the heterodimer comprises the LILRB polypeptide and a second monomer comprising an amino acid sequence of a protein selected from the group consisting of SIRPα, PD1, TIGIT and SIGLEC10, wherein the amino acid sequence is capable of binding it's natural binding pair.

According to some embodiments of the invention, a first monomer of the heterodimer comprises the LILRB2 polypeptide and a second monomer comprising an amino acid sequence of a protein selected from the group consisting of SIRPα, PD1, TIGIT and SIGLEC10, wherein the amino acid sequence is capable of binding it's natural binding pair.

According to some embodiments of the invention, a first monomer of the heterodimer comprises the LILRB polypeptide and a second monomer comprising an amino acid sequence of SIRPα, wherein the amino acid sequence is capable of binding CD47.

According to some embodiments of the invention, a first monomer of the heterodimer comprises the LILRB2 polypeptide and a second monomer comprising an amino acid sequence of SIRPα, wherein the amino acid sequence is capable of binding CD47.

According to an aspect of some embodiments of the present invention there is provided a heterodimer comprising a first monomer comprising the fusion polypeptide and a second monomer comprising an amino acid sequence of SIRPα attached to an Fc domain of an antibody or a fragment thereof, wherein the amino acid sequence of SIRPα is capable of binding CD47.

According to some embodiments of the invention, the SIRPα amino acid sequence is at least 90% identical to SEQ ID NO: 27 or 88.

According to some embodiments of the invention, the SIRPα amino acid sequence comprises SEQ ID NO: 27 or 88.

According to some embodiments of the invention, the SIRPα amino acid sequence is as set forth in SEQ ID NO: 27 or 88.

According to an aspect of some embodiments of the present invention there is provided a composition comprising the dimer, wherein the dimer is the predominant form of the LILRB in the composition.

According to an aspect of some embodiments of the present invention there is provided a composition comprising the dimer, wherein the dimer is the predominant form of the LILRB2 in the composition.

According to an aspect of some embodiments of the present invention there is provided a polynucleotide encoding the LILRB polypeptide, the fusion polypeptide or the dimer.

According to an aspect of some embodiments of the present invention there is provided a polynucleotide encoding the LILRB2 polypeptide, the fusion polypeptide or the dimer.

According to an aspect of some embodiments of the present invention there is provided a nucleic acid construct comprising a polynucleotide encoding the LILRB polypeptide, the fusion polypeptide or the dimer, and a regulatory element for directing expression of the polynucleotide in a host cell.

According to an aspect of some embodiments of the present invention there is provided a nucleic acid construct comprising a polynucleotide encoding the LILRB2 polypeptide, the fusion polypeptide or the dimer, and a regulatory element for directing expression of the polynucleotide in a host cell.

According to an aspect of some embodiments of the present invention there is provided a host cell comprising the LILRB polypeptide, the fusion polypeptide or the dimer, the polynucleotide or the nucleic acid construct.

According to an aspect of some embodiments of the present invention there is provided a host cell comprising the LILRB2 polypeptide, the fusion polypeptide or the dimer, the polynucleotide or the nucleic acid construct.

According to an aspect of some embodiments of the present invention there is provided a method of producing a polypeptide, the method comprising introducing the polynucleotide or the nucleic acid construct to a host cell or culturing the cells.

According to some embodiments of the invention, the method comprising isolating the LILRB polypeptide, the fusion polypeptide or the dimer.

According to some embodiments of the invention, the method comprising isolating the LILRB2 polypeptide, the fusion polypeptide or the dimer.

According to an aspect of some embodiments of the present invention there is provided a method of treating a disease associated with pathologic cells expressing HLA-G in a subject in need thereof, the method comprising administering to the subject a therapeutically effective amount of the LILRB polypeptide, the composition, the fusion polypeptide or the dimer, the polynucleotide, the nucleic acid construct or the host cell, thereby treating the disease in the subject.

According to an aspect of some embodiments of the present invention there is provided a method of treating a disease associated with pathologic cells expressing HLA-G in a subject in need thereof, the method comprising administering to the subject a therapeutically effective amount of the LILRB2 polypeptide, the composition, the fusion polypeptide or the dimer, the polynucleotide, the nucleic acid construct or the host cell, thereby treating the disease in the subject.

According to an aspect of some embodiments of the present invention there is provided the LILRB polypeptide, the composition, the fusion polypeptide or the dimer, the polynucleotide, the nucleic acid construct or the host cell, for use in treating a disease associated with pathologic cells expressing HLA-G in a subject in need thereof.

According to an aspect of some embodiments of the present invention there is provided the LILRB2 polypeptide, the composition, the fusion polypeptide or the dimer, the polynucleotide, the nucleic acid construct or the host cell, for use in treating a disease associated with pathologic cells expressing HLA-G in a subject in need thereof.

According to some embodiments of the invention, the disease is cancer.

According to some embodiments of the invention, the cancer is selected from the group consisting of pancreatic, breast, skin, colorectal, gastric and ovarian cancer.

According to an aspect of some embodiments of the present invention there is provided a method of activating immune cells, the method comprising in-vitro activating immune cells in the presence of the LILRB polypeptide, the composition, the fusion polypeptide or the dimer, the polynucleotide, the nucleic acid construct or the host cell.

According to an aspect of some embodiments of the present invention there is provided a method of activating immune cells, the method comprising in-vitro activating immune cells in the presence of the LILRB2 polypeptide, the composition, the fusion polypeptide or the dimer, the polynucleotide, the nucleic acid construct or the host cell.

According to some embodiments of the invention, the activating is in the presence of cells expressing HLA-G.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWING(S)

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

### In the Drawings:

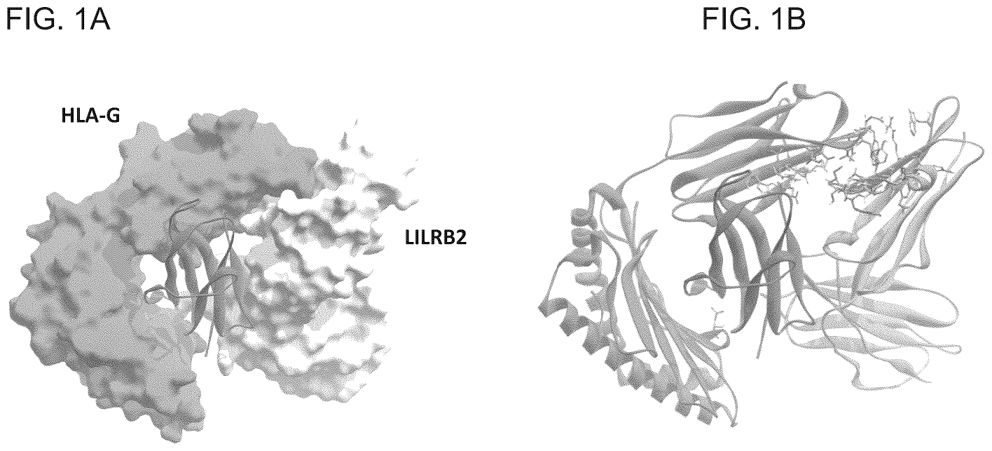

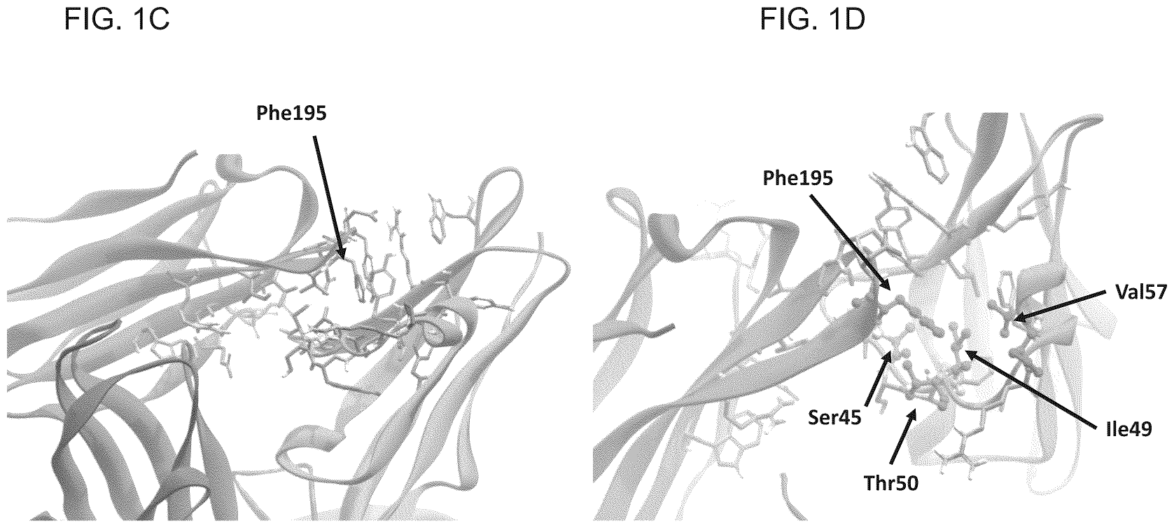

FIGS. 1A-D demonstrate structural analysis of LILRB2-HLA-G binding interface.

FIG. 1A shows the PDB ID-2DYP Complex structure of LILRB2 (SEQ ID NO: 1, also referred to herein as “WT LILRB2”) and HLA-G (SEQ ID NO: 3). HLA-G is shown in dark-grey surface representation, beta-2-microglobulin (SEQ ID NO: 4) is shown in grey ribbons display and LILRB2 is shown in white surface representation. FIG. 1B shows mapping of the interface residues between HLA-G and LILRB2. Both HLA-G and beta-2-microglobulin are shown in dark-grey ribbons and LILRB2 is shown in grey ribbons. The interacting residues are represented with balls and sticks. FIG. 1C shows zooming into the interaction interface between HLA-G and LILRB2 shown in FIG. 1B. In the interacting interface, only a single dominant difference exists between HLA-G and its homologues at PHE195. FIG. 1D shows four amino acids in LILRB2, namely Ser45, Ile49, Thr50 and Val57, which were found to have significant influence on binding energy.

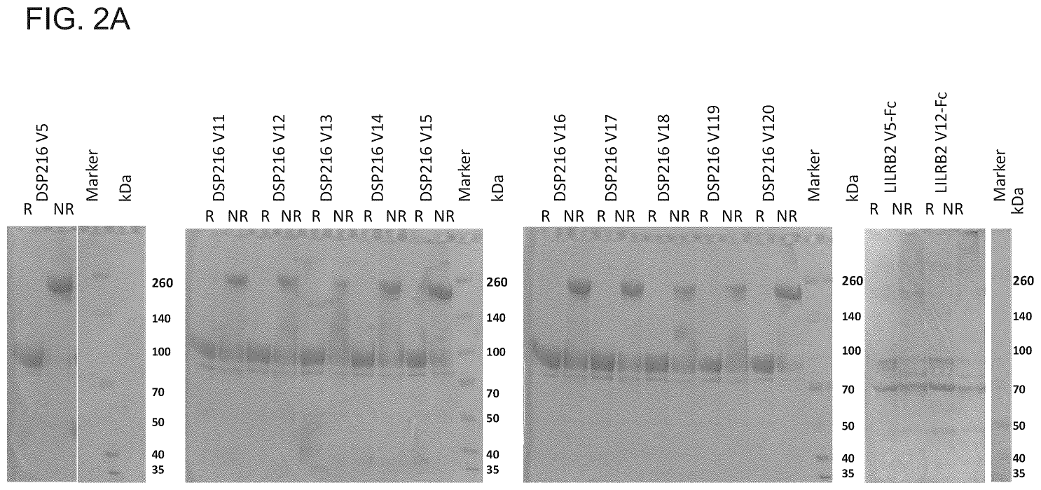

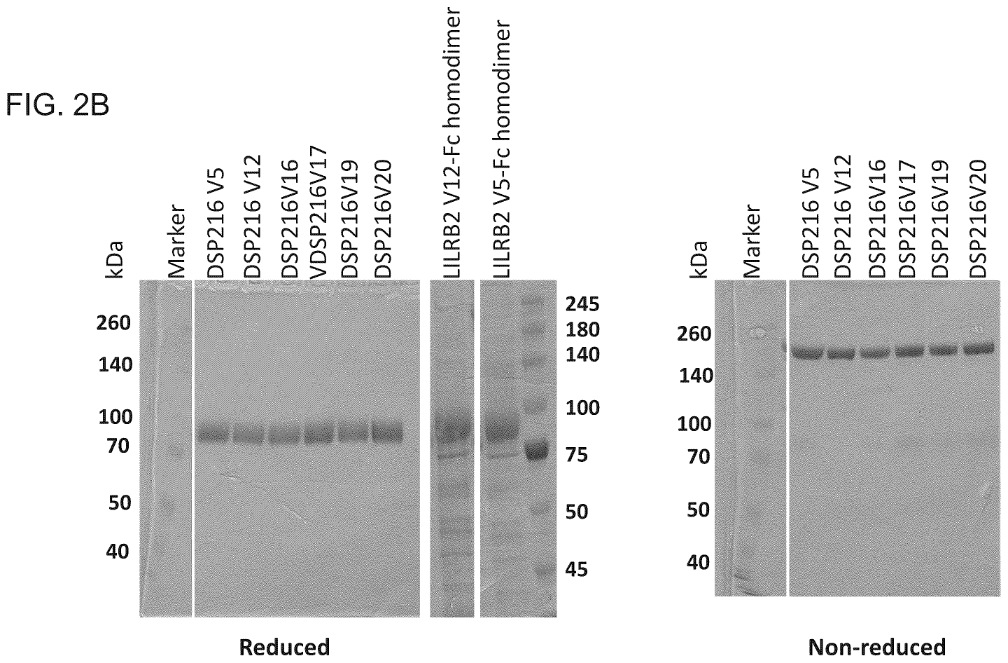

FIGS. 2A-B show photographs of SDS poly acrylamide gel electrophoresis (SDS-PAGE) analysis of several heterodimer proteins comprising either a WT LILRB2 (SEQ ID NO: 1) or a mutated LILRB2 (referred to herein as “LILRB2 variant”)-Fc fusion and a SIRPα-Fc fusion (see Table 3 hereinbelow for full description of each heterodimer), separated under reducing and/or non-reducing conditions. The samples presented in the figures are of crude (non-purified, FIG. 2A) or protein-A purified (FIG. 2B)-five days-supernatant. The supernatants are from Expi293F cells that were transfected with plasmids encoding the recombinant proteins as indicated.

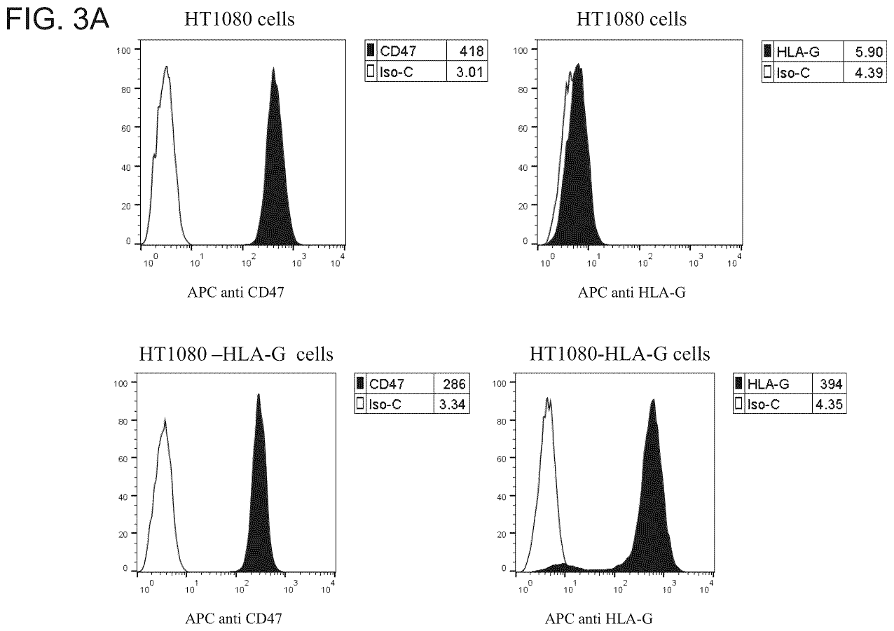

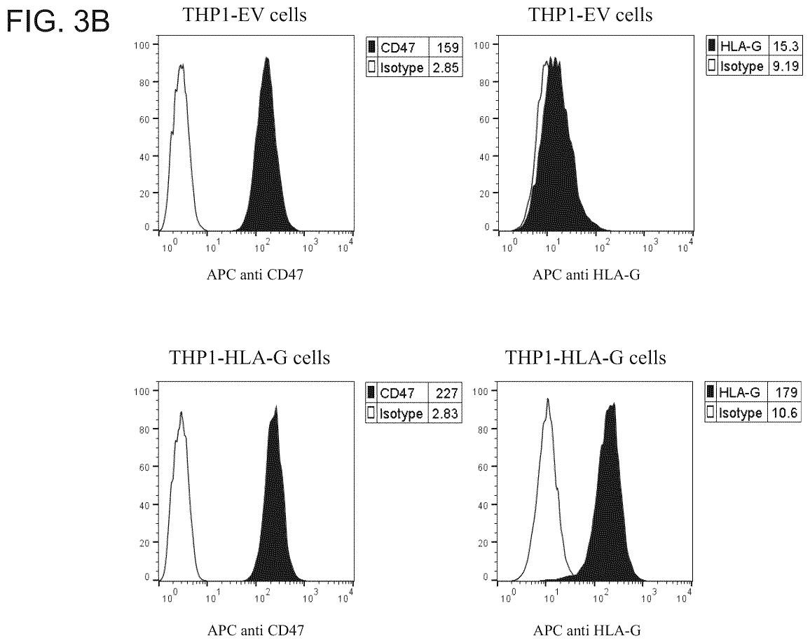

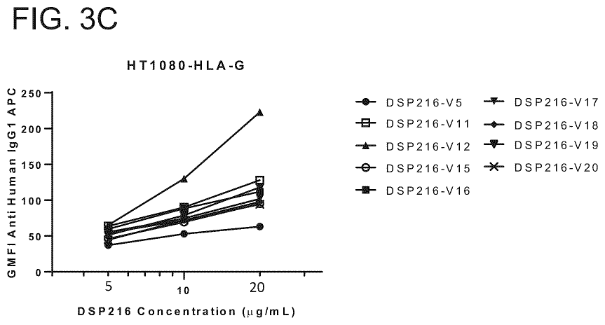

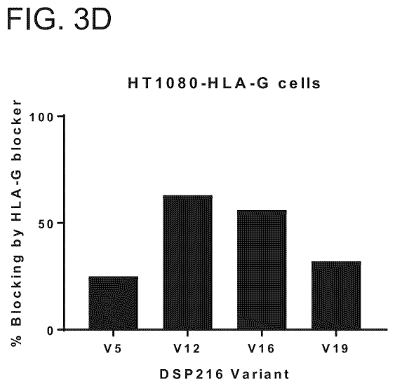

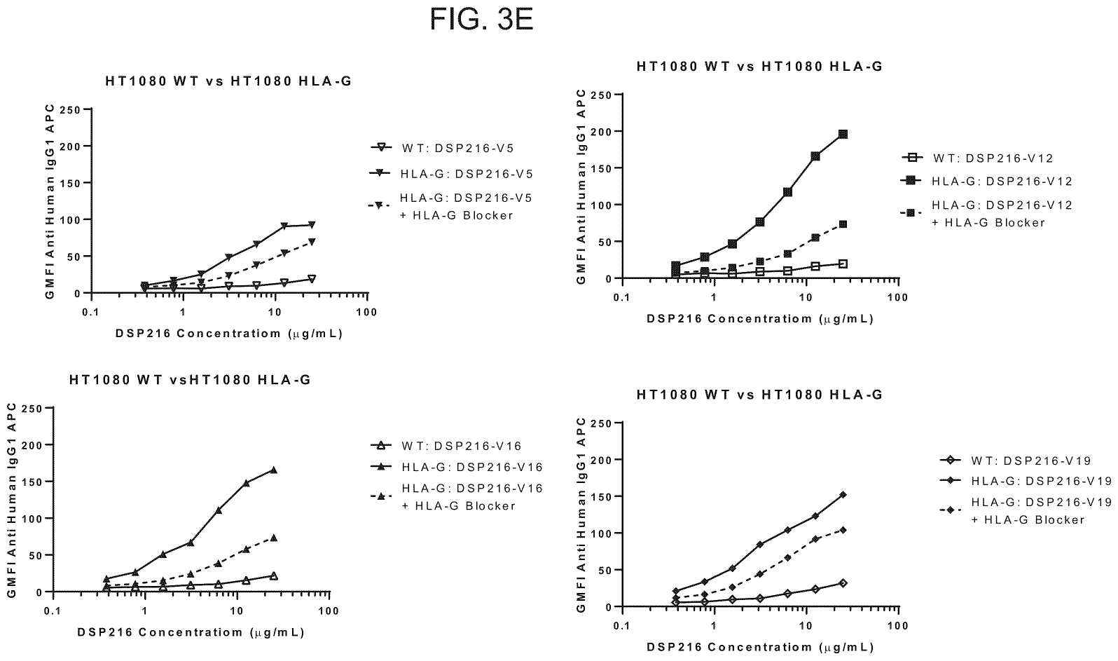

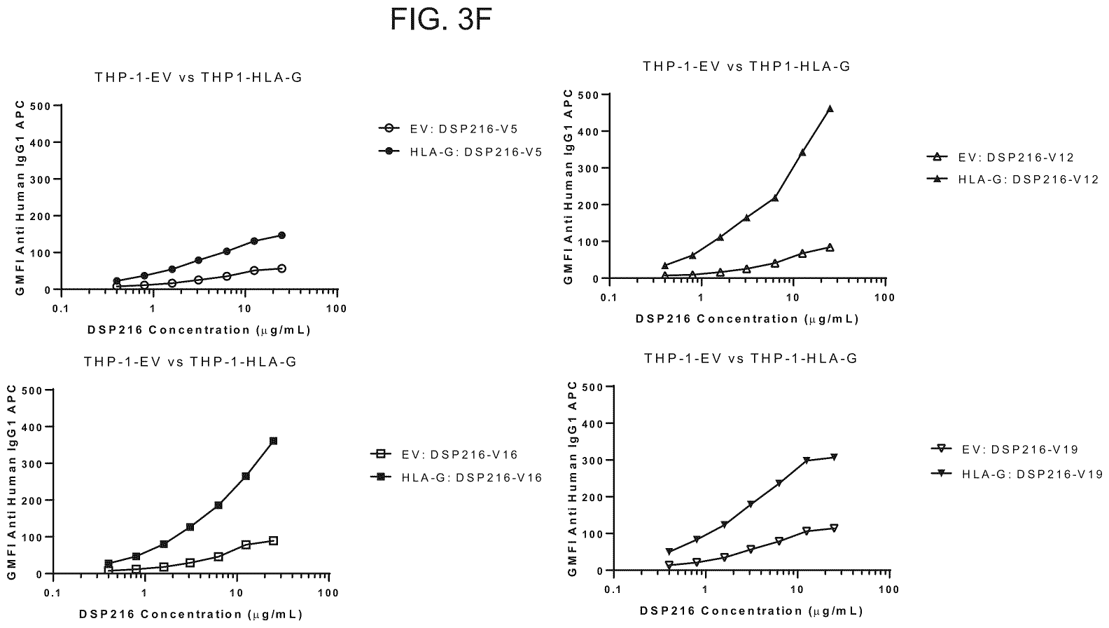

FIGS. 3A-F demonstrate binding of several heterodimer proteins comprising the LILRB2 variants to HLA-G expressed on the cell surface as compared WT LILRB2. FIGS. 3A-B demonstrate the expression levels of CD47 and HLA-G on HT1080 and HT1080-HLA-G cells (FIG. 3A) and THP1-EV and THP1-HLA-G cells (FIG. 3B). The cell surface expression levels of CD47 and HLA-G were determined by immune-staining of the cell lines with an anti-human-CD47 antibody and IgG1 as an isotype control or anti-human HLA-G antibody and IgG4 as an isotype control, followed by flow cytometry analysis. FIG. 3C demonstrates binding of the indicated heterodimers to the HLA-G overexpressing cells HT1080-HLA-G. Binding was determined following incubation, by immune-staining of the IgG backbone using an anti-human-IgG1 antibody, followed by flow cytometry analysis. GMFI values were used to create the binding curve graphs with the GraphPad Prism software. FIG. 3D demonstrates the blocking percentages of the indicated heterodimers' binding to HT1080-HLA-G cells by an anti-HLA-G blocking antibody. FIGS. 3E-F demonstrates binding of the indicated heterodimers to HT1080 cells or to the HLA-G overexpressing cells HT1080-HLA-G (FIG. 3E) or to THP-1-EV cells or the HLA-G overexpressing cells THP-1-HLA-G (FIG. 3F) with or without a blocking antibody, as indicated. Binding was determined following incubation, by immune-staining of the IgG backbone using an anti-human-IgG1 antibody, followed by flow cytometry analysis. GMFI values were used to create the binding curve graphs with the GraphPad Prism software.

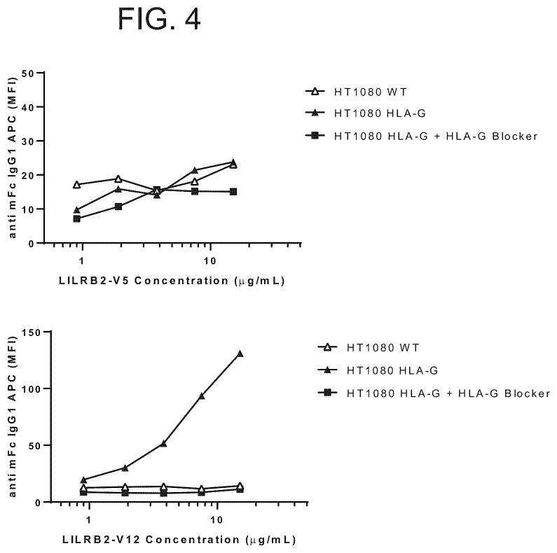

FIG. 4 demonstrates binding of a homodimer protein comprising the LILRB2 variant LILRB-V12 to HLA-G expressed on the cell surface as compared to a homodimer protein comprising the WT LILRB2 (LILRB2-V5). Binding of the homodimers to HT1080 cells or to the HLA-G overexpressing cells HT1080-HLA-G cells, with or without a blocking antibody, was determined following incubation by immune-staining of the IgG backbone using anti human-IgG1 antibody, followed by flow cytometry analysis. GMFI values are presented and were used to create binding curve graphs with the GraphPad Prism software.

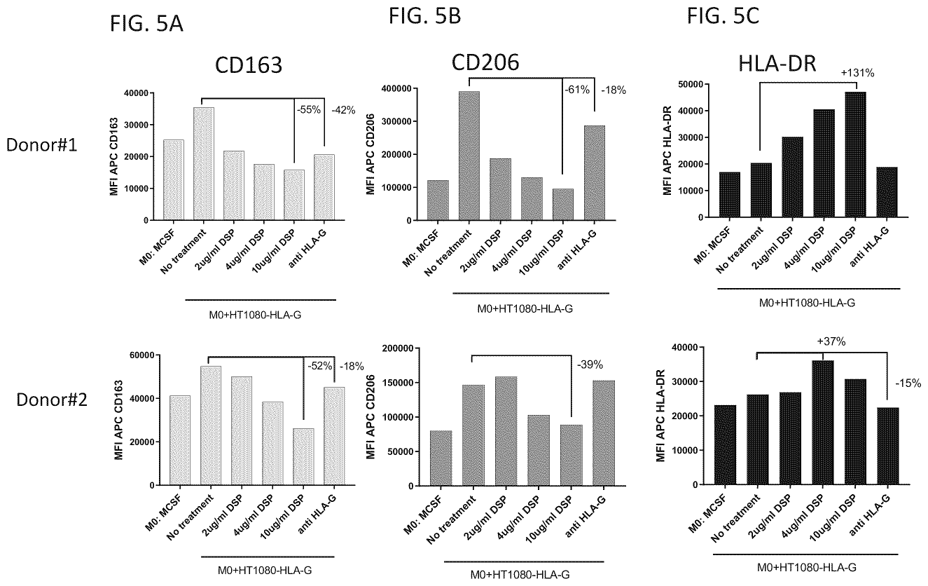

FIGS. 5A-C demonstrate the expression levels of the M2 markers, CD163 (FIG. 5A) and CD206 (FIG. 5B); and the M1 marker HLA-DR (FIG. 5C), in M-CSF treated macrophages co-cultured with HT1080-HLA-G cells and treated with the LILRB2 variant-Fc fusion and a SIRPα-Fc fusion heterodimer referred to herein as “DSP216-V12” (marked on the Figures as “DSP”), see Table 3 hereinbelow for full description of each heterodimer) at the indicated concentrations or with 1.5 μg/ml anti HLA-G antibody as a positive control. The cell surface expression levels were determined by immunostaining of the cells with anti-human-CD163, anti-human-CD206 or anti-human HLA-DR antibodies, followed by flow cytometry analysis. MFI values were used to create the binding curve graphs with the GraphPad Prism software.

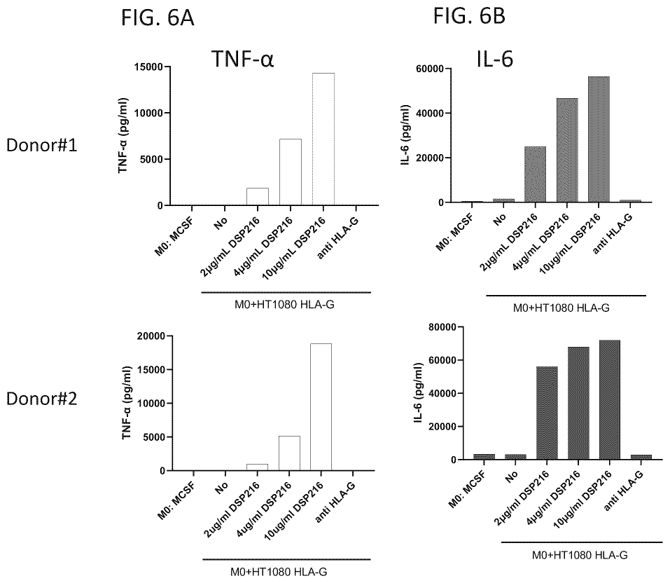

FIGS. 6A-B demonstrate the levels of TNF-α (FIG. 6A) or IL-6 (FIG. 6B) in supernatants of MCS-F treated macrophages and HT1080 HLA-G cells co-cultures, 24 hours following beginning of the co-culture and treatment with DSP216-V12 (marked on the Figures as “DSP2016”) at the indicated concentrations or 1.5 μg/ml anti HLA-G antibody as a positive control. The cytokine levels (ng/mL) were determined by immunostaining of the supernatants with Cytometric Bead Array (CBA), followed by flow cytometry analysis. MFI values were used to create the binding curve graphs with the GraphPad Prism software.

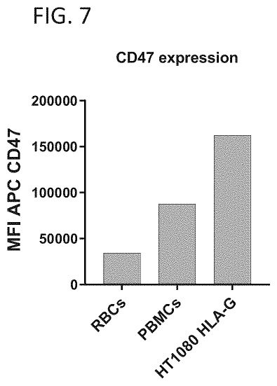

FIG. 7 demonstrates the expression levels of CD47 on RBCs, PBMCs or on HT1080-HLA-G cells. The cell surface expression levels were determined by immunostaining of the cells with an anti-human-CD47 antibody or IgG1 as an isotype control, followed by flow cytometry analysis. MFI values were used to create the binding curve graphs with the GraphPad Prism software.

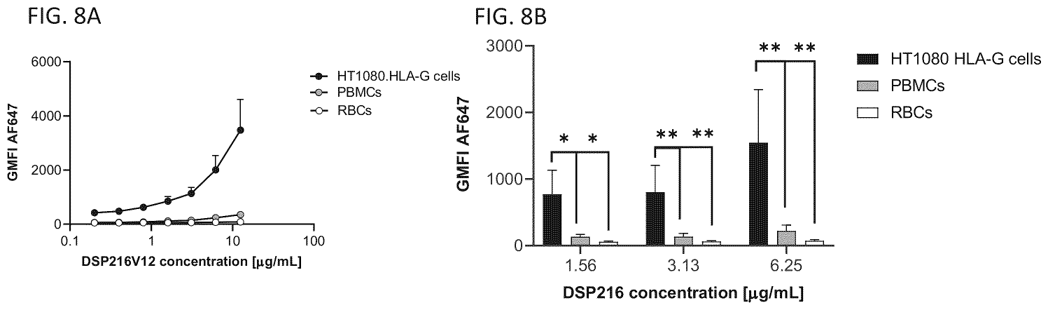

FIGS. 8A-B demonstrate binding of DSP216-V12 to RBCs, PBMCs or HT1080-HLA-G cells, as determined by flow cytometry. The graphs represent the average (±SEM) of the MFI from four independent donor samples. FIG. 8A is a graph showing the average (±SEM) of binding of DS216-V12 at concentrations of 0.4-12.5 μg/mL to RBCs, PBMCs or HT1080-HLA-G cells. FIG. 8B is a bar graph showing the significantly higher binding of DSP216-V12 (marked on the Figure as “DSP2016”) to HT1080-HLA-G cells as compared to PBMCs and RBCs at all concentrations (1.56, 3.125, 6.25 μg/mL). *P≤0.05, **P≤0.01. P values for the 3.125 μg/mL concentration were 0.0044 vs. PBMCs and 0.0027 vs. RBCs (T-Test).

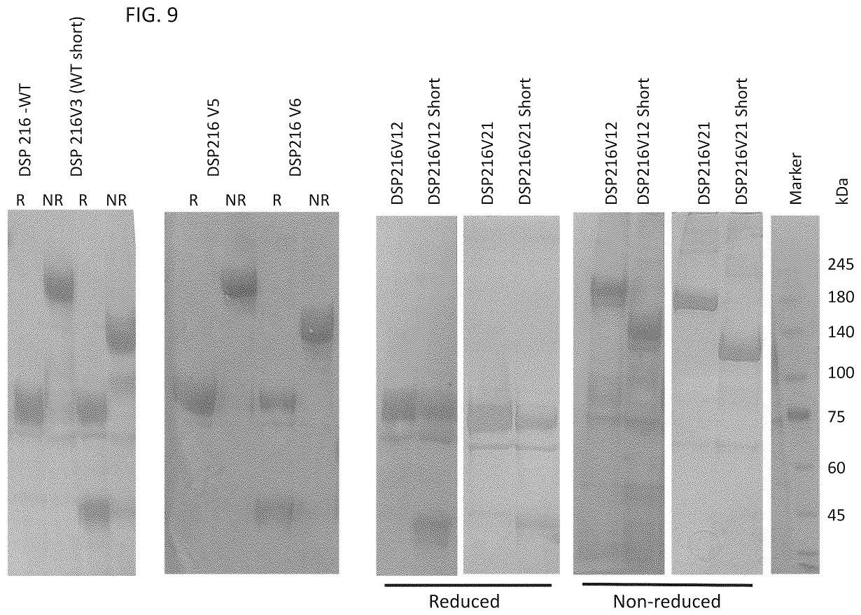

FIG. 9 shows photographs of SDS poly acrylamide gel electrophoresis (SDS-PAGE) analysis of several heterodimer proteins comprising a SIRPα-Fc fusion subunit and a WT LILRB2- or LILRB2 variant-Fc fusion subunit (see Table 3 hereinbelow for full description of each heterodimer indicated in the Figure), separated under reducing and non-reducing conditions. The samples presented in the figures are of crude (non-purified-five days-supernatant). The supernatants are from Expi293F cells that were transfected with plasmids encoding the recombinant proteins as indicated.

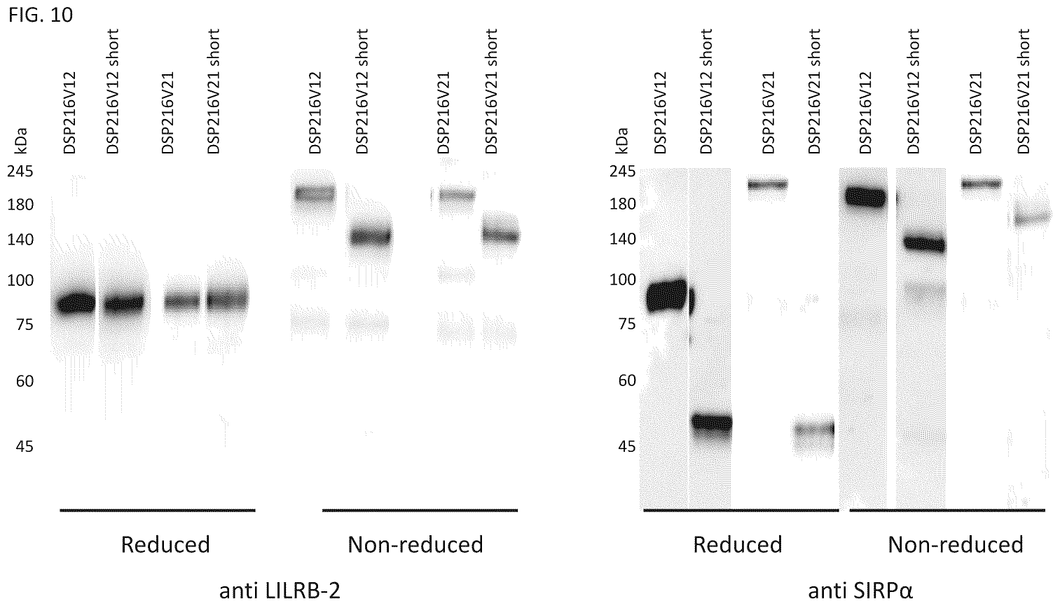

FIG. 10 are photographs of Western Blot analysis of several heterodimer proteins comprising a SIRPα-Fc fusion subunit and a LILRB2 variant-Fc fusion subunit (see Table 3 hereinbelow for full description of each heterodimer indicated in the Figure). The samples presented in the figures are of protein A purified-five days-supernatant. The supernatants are from Expi293F cells that were transfected with plasmids encoding to the heterodimers as indicating. The proteins were separated on SDS-PAGE under reducing and non-reducing conditions, followed by immunoblotting with anti-LILRB2 or anti-SIRPα antibodies.

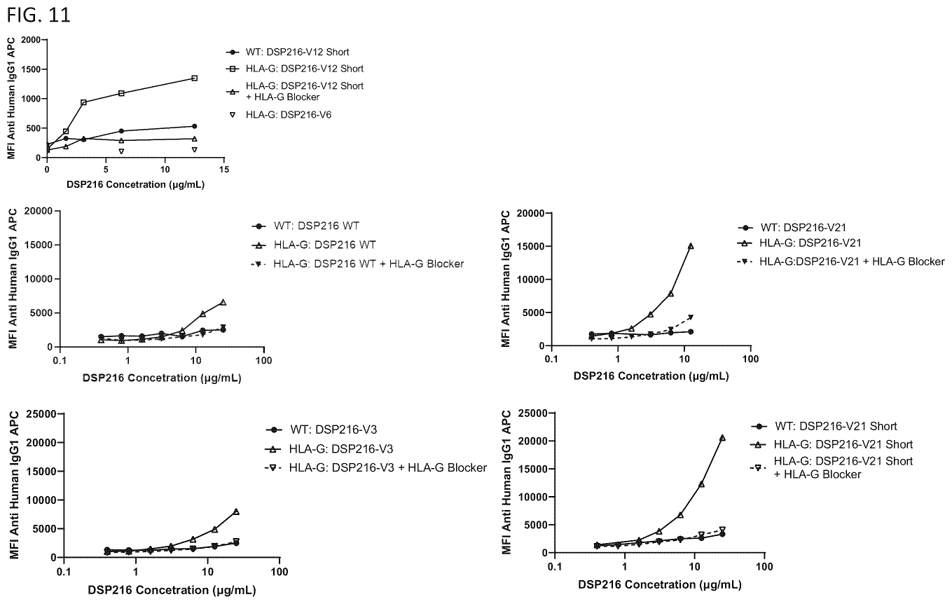

FIG. 11 demonstrates binding of several heterodimer proteins comprising a SIRPα-Fc fusion subunit and a WT LILRB2- or LILRB2 variant-Fc fusion subunit (see Table 3 hereinbelow for full description of each heterodimer indicated in the Figure) to CD47 expressed on the cell surface of HT1080 or to CD47 and HLA-G expressed on the cell surface of HT1080-HLA-G cells overexpressing HLA-G. The binding with or without anti HLA-G (an HLA-G/LILRB2 interacting-blocking antibody) demonstrate binding to CD47 only compared to binding to both HLA-G and CD47. Binding was determined following immunostaining of the IgG backbone using anti human-IgG1 antibody, followed by flow cytometry analysis. GMFI values are presented and were used to create binding curve graphs with the GraphPad Prism software.

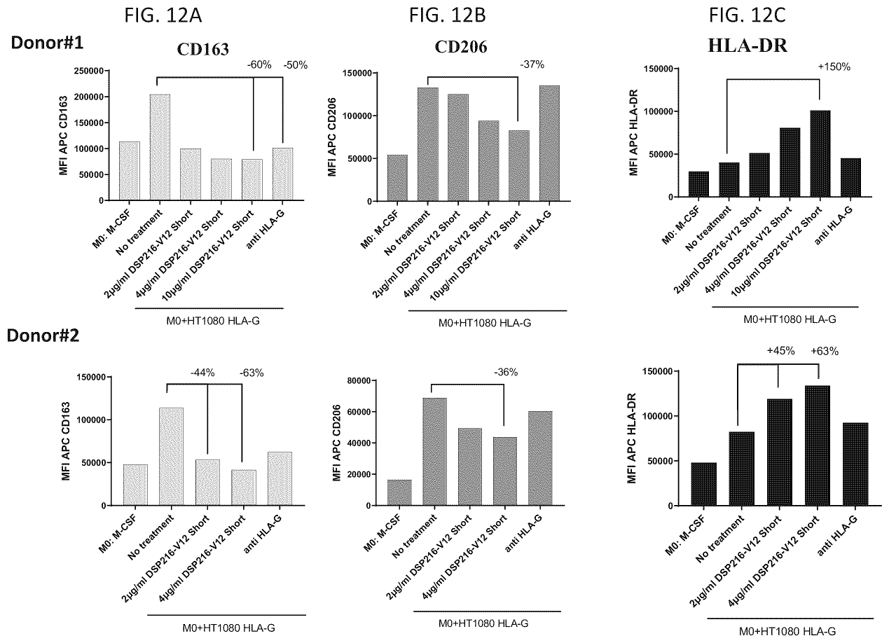

FIGS. 12A-C demonstrate the cell surface expression levels of the M2 markers, CD163 (FIG. 12A) and CD206 (FIG. 12B); and the M1 marker HLA-DR (FIG. 12C), in M-CSF treated macrophages co-cultured with HT1080-HLA-G cells and treated with the LILRB2 variant-Fc fusion and a short SIRPα-Fc fusion heterodimer referred to herein as “DSP216-V12 short” (see Table 3 hereinbelow for full description of the heterodimer) at the indicated concentrations or with 1.5 μg/ml anti HLA-G antibody as a control. The cell surface expression levels were determined by immunostaining of the cells with anti-human-CD163, anti-human-CD206 or anti-human HLA-DR antibodies, followed by flow cytometry analysis. MFI values were used to create the binding curve graphs with the GraphPad Prism software.

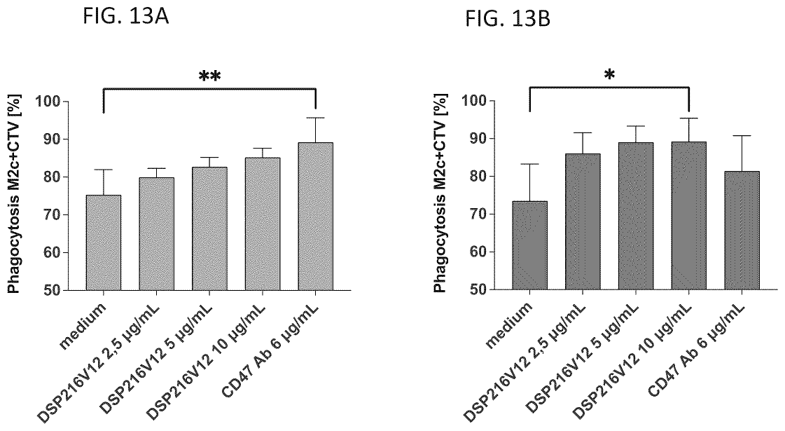

FIGS. 13A-B demonstrate phagocytosis of cancer cells treated with DSP216-V12 or an anti-CD47 antibody by M2c macrophages as determined by flow cytometry. The graphs show average % (±SD) of M2c cells that are positive for cell trace violet after uptake of stained cancer cells. The graphs show the average uptake by M2c macrophages of CD47⁺ HLA-G-721.221 cells that were transduced with empty vector (721.221EV) (FIG. 13A) or CD47⁺ HLA-G⁺721.221 cells that were transduced with HLA-G expressing vector (721.221-HLA-G) (FIG. 13B). Both 721.221 cell lines were incubated in a medium containing different concentrations of DSP216-V12 or 6 μg/mL CD47 blocking antibody prior to the mixing with the M2c macrophages. *P≤0.05, **P≤0.01. P value for the % phagocytosis in medium vs. 6 μg/mL CD47 antibody treatments, presented in FIG. 13A=0.0028 and for % phagocytosis in medium vs. 10 μg/mL DSP216-V12 treatments, presented in FIG. 13B=0.0465 (paired T-Test).

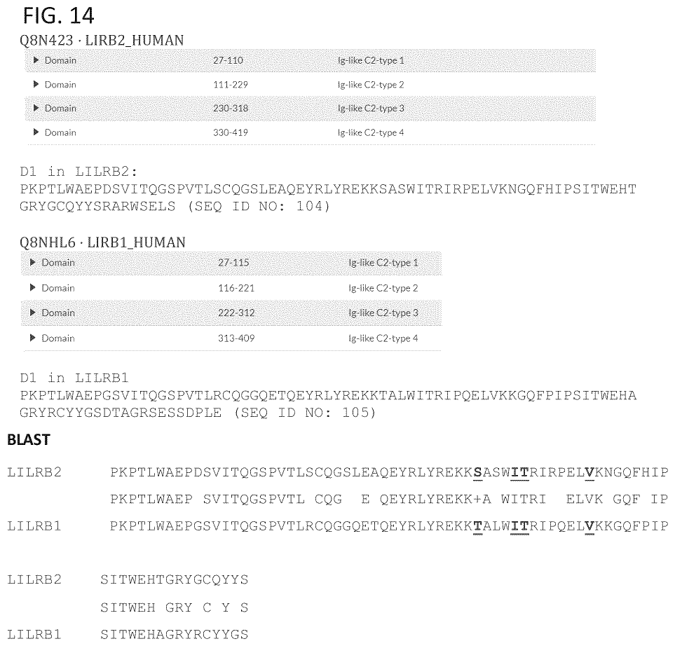

FIG. 14 shows blast homology analysis between LILRB2 and LILRB1.

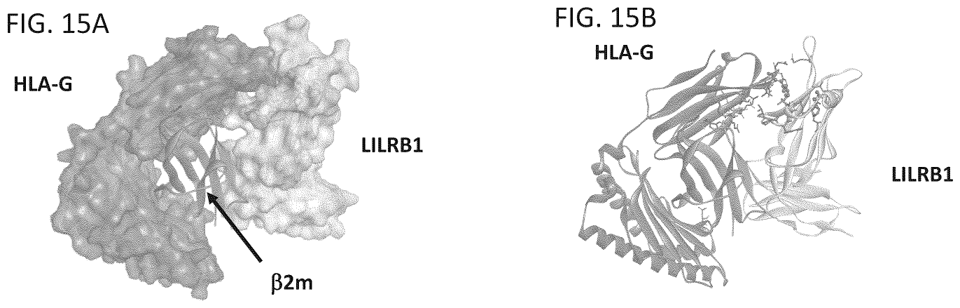

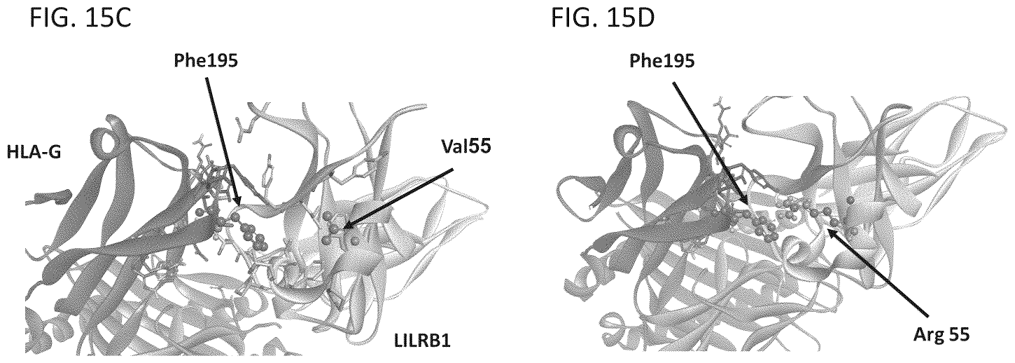

FIGS. 15A-D demonstrate structural analysis of LILRB1-HLA-G binding interface. FIG. 15A shows the PDB ID-2DYP Complex structure of LILRB1 (SEQ ID NO: 102, also referred to herein as “WT LILRB1”) and HLA-G (SEQ ID NO: 3). HLA-G is shown in grey surface representation, beta-2-microglobulin (SEQ ID NO: 4) is shown in dark-grey ribbons display and LILRB1 is shown in white surface representation. FIG. 15B shows mapping of the interface residues between HLA-G and LILRB1. Both HLA-G and beta-2-microglobulin are shown in dark-grey ribbons and LILRB1 is shown in grey ribbons. The interacting residues are represented with balls and sticks. FIG. 15C shows zoom-in into the interaction interface between HLA-G and LILRB1 shown in FIG. 15B, specifically demonstrating Val55 of LILRB1 and Phe 195 of HLA-G. FIG. 15D demonstrates the proximity of Phe195 of HLA-G to amino acid residue 55 of LILRB1 following substituting the WT Valine to Arginine (the variant LILRB1 sequence is shown in SEQ ID NO: 103).

### DESCRIPTION OF SPECIFIC EMBODIMENTS OF THE INVENTION

The present invention, in some embodiments thereof, relates to LILRB polypeptides and uses thereof.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

HLA-G, a non-classical MHC class I molecule, was first known to confer protection to the fetus from destruction by the immune system of its mother, thus critically contributing to fetal-maternal tolerance. The reported receptors for HLA-G include LILRB1, LILRB2 and KIR2DL4. While HLA-G is predominantly expressed on placenta trophoblasts and thymic epithelial cells, it has been shown that cells associated with various pathological conditions, including e.g. cancer, viral infections, auto-immune and inflammatory diseases or after allo-transplantation, overexpress HLA-G; and in-fact HLA-G-LILRB1/2 signaling has been suggested as an immune escape mechanism employed by pathologic cells.

Whilst reducing specific embodiments of the present invention to practice the present inventors have employed structural-functional tools to introduce mutations in LILRB2 and LILRB1 which aim at increasing stability and binding affinity to HLA-G (Examples 1 and 11 of the Examples section which follows). Using this methodology, the present inventors were able to generate novel polypeptides having mutations within a specific region in the D1 domain of the protein having improved stability and binding affinity to HLA-G as compared to wild-type LILRB2 (Examples 1-9 of the Examples section which follows).

Consequently, specific embodiments of the present teachings suggest LILRB (e.g. LILRB2, LILRB1) polypeptides having these novel mutations; and their use in therapy.

Thus, according to an aspect of the present invention, there is provided a LILRB polypeptide capable of binding an HLA-G polypeptide as set forth in SEQ ID NO: 3 and having at least one mutation located within amino acid positions 40-60 of a D1 domain of LILRB, wherein said LILRB polypeptide has an increased stability and/or increased affinity to said HLA-G compared to a LILRB polypeptide of the same length and sequence not comprising said at least one mutation.

According to an additional or an alternative aspect of the present invention, there is provided a LIL2B2 polypeptide capable of binding an HLA-G polypeptide as set forth in SEQ ID NO: 3 and having at least one mutation at an amino acid position selected from the group consisting of S45, I49, T50 and V57 corresponding to SEQ ID NO: 1, wherein said LILRB2 polypeptide has an increased stability and/or increased affinity to said HLA-G compared to a LILRB2 polypeptide of the same length and sequence not comprising said at least one mutation.

“LILRB (Leukocyte immunoglobulin-like receptor subfamily B)” refers to a family of receptors comprising 2-4 extracellular immunoglobulin-like domains (specifically, C-type Ig-like domains, InterPro database entry IPR008424 or Pfam database entry PF05790) and a cytoplasmic tail containing an ITIM domain including LILRB1, LILRB2, LILRB3, LILRB4 and LILRB5.

According to specific embodiments, the LILRB is a human LILRB.

According to specific embodiments, the LILRB is LILRB2.

“LILRB2 (Leukocyte immunoglobulin-like receptor subfamily B member 2)” refers to the polypeptide encoded by the LILRB2 gene (corresponding to human Gene ID 10288). According to specific embodiments, LILRB2 is human LILRB2. According to a specific embodiment, the LILRB2 refers to the human LILRB2, such as provided in the following GenBank Number NP_001074447, NP_001265332, NP_001265333, NP_001265334, NP_001265335 or the UniProt Number Q8N423.

According to specific embodiments, the LILRB is LILRB1.

“LILRB1 (Leukocyte immunoglobulin-like receptor subfamily B member 1)” refers to the polypeptide encoded by the LILRB1 gene (corresponding to human Gene ID 10859). According to specific embodiments, LILRB1 is human LILRB1. According to a specific embodiment, the LILRB1 refers to the human LILRB1, such as provided in the following GenBank Number NP_001075106, NP_001075107, NP_001075108, NP_001265327, NP_001265328 or the UniProt Number Q8NHL6.

One of the known binding pairs of LILRB (e.g. LILRB2, LILRB1) is a major histocompatibility molecule (MHC, e.g. HLA-G).

“HLA-G (human leukocyte antigen G)” refers to the polypeptide encoded by the HLA-G gene (Gene ID 3135). According to a specific embodiment, HLA-G amino acid sequence is as provided in SEQ ID NO: 3.

According to specific embodiments, the LILRB (e.g. LILRB2, LILRB1) binds HLA-G in the context of beta2-microglobulin. A non-limiting example of beta2-microglobulin sequence is provided is SEQ ID NO: 4.

According to specific embodiments, the LILRB (e.g. LILRB2, LILRB1) binds free HLA-G or HLA-G not in the context of non-beta2-microglobulin.

According to specific embodiments, the LILRB (e.g. LILRB2, LILRB1) binds a free HLA-G (i.e. not bound to a peptide).

According to specific embodiments, the LILRB (e.g. LILRB2, LILRB1) binds an HLA-G presenting a peptide.

According to specific embodiments, the LILRB (e.g. LILRB2, LILRB1) binds the HLA-G independent of the peptide sequence.

Assays for testing binding are well known in the art and include, but not limited to flow cytometry, BiaCore, bio-layer interferometry Blitz® assay, HPLC.

As used herein, the terms “LILRB polypeptide”, “LILRB2 polypeptide” and “LILRB1 polypeptide” refer to full-length LILRB, LILRB2 and LILRB1, functional fragments thereof or homologs thereof which maintain at least the ability to bind HLA-G. For example, according to specific embodiments, the amino acid sequence of the polypeptide comprises a substitution, addition and/or deletion mutation as compared to the sequence of the wild type protein, as further described herein.

According to specific embodiments, the amino acid sequence of the LILRB2 polypeptide comprises substitution, addition and deletion mutations (e.g. as compared to a LILRB2 polypeptide as set forth in SEQ ID NO: 1) as further described hereinabove and below.

According to specific embodiments, the amino acid sequence of the LILRB1 polypeptide comprises substitution, addition and deletion mutations (e.g. as compared to a LILRB1 polypeptide as set forth in SEQ ID NO: 102) as further described hereinabove and below.

According to specific embodiments, the LILRB (e.g. LILRB2, LILRB1) polypeptide comprises an extracellular domain of LILRB (e.g. LILRB2, LILRB1) or a functional fragment thereof capable of at least binding HLA-G.

As noted hereinabove, the extracellular domain of LILRBs comprises 2-4 immunoglobulin-like domains which are known in the art as D domains and are marked from 1-4 by their position from distal-to-proximal relative to the membrane (which also corresponds to their order from N to C on the amino acid sequence of the protein).

The extracellular domain of LILRB2 or LILRB1 comprises 4 Ig-like domains, known as D1-D4. Non-limiting examples of LILRB2 and LILRB1 extracellular domains are provided in SEQ ID Nos: 1, 100 and 102.

Hence, according to specific embodiments, the amino acid sequence of LILRB (e.g. LILRB2, LILRB1) polypeptide comprises at least one Ig-like domain or a functional fragment thereof.

According to specific embodiments, the amino acid sequence of LILRB (e.g. LILRB2, LILRB1) polypeptide comprises at least one Ig-like domain.

According to specific embodiments, the LILRB (e.g. LILRB2, LILRB1) polypeptide comprises at least two Ig-like domains, at least three Ig-like domains or four Ig-like domains or functional fragments thereof.

According to specific embodiments, the LILRB (e.g. LILRB2, LILRB1) polypeptide comprises at least two Ig-like domains, at least three Ig-like domains or four Ig-like domains.

According to specific embodiments, the LILRB (e.g. LILRB2, LILRB1) polypeptide comprises at least the D1 domain.

According to specific embodiments, the LILRB2 polypeptide comprises domains D1 and D2 of LILRB2; domains D1, D2 and D3 of LILRB2, domains D1, D2 and D4 or LILRB2, or domains D1, D2, D3 and D4 of LILRB2.

According to specific embodiments, the LILRB1 polypeptide comprises domains D1 and D2 of LILRB1; domains D1, D2 and D3 of LILRB1, domains D1, D2 and D4 or LILRB1, or domains D1, D2, D3 and D4 of LILRB1.

According to specific embodiments, the LILRB (e.g. LILRB2, LILRB1) polypeptide comprises at least 70, at least 80, at least 90, at least 100 amino acids, each possibility represents a separate embodiment of the present invention.

According to specific embodiments, LILRB (e.g. LILRB2, LILRB1) polypeptide comprises 100-597 amino acids, 100-500 amino acids, 100-400 amino acids, 150-400 amino acids, 300-400 amino acids, 350-400 amino acids, 150-250 amino acids, each possibility represents a separate embodiment of the present invention.

The terms “LILRB polypeptide”, “LILRB2 polypeptide” and “LILRB1 polypeptide” also encompass functional homologues which exhibit the desired activity (i.e., binding MHC, e.g. HLA-G). Such homologues can be, for example, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identical or homologous to the amino acid sequences of LILRB, LILRB2 and LILRB1 that are described herein; or at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identical to the polynucleotide sequence encoding same (as further described hereinbelow).

As used herein, “identity” or “sequence identity” refers to global identity, i.e., an identity over the entire amino acid or nucleic acid sequences disclosed herein and not over portions thereof.

Sequence identity or homology can be determined using any protein or nucleic acid sequence alignment algorithm such as Blast, ClustalW, and MUSCLE.

The homolog may also refer to an ortholog, a deletion, insertion, or substitution variant, including an amino acid substitution, as further described hereinbelow.

According to specific embodiments, the LILRB (e.g. LILRB2, LILRB1) polypeptide may comprise conservative and/or non-conservative amino acid substitutions (also referred to herein as “mutations”), as further described in details hereinbelow.

The LILRB polypeptides described herein comprise at least one mutation in the D1 domain of LILRB. Non-limiting examples of D1 domains of LILRB2 and LILRB1 are provided in SEQ ID NO: 104 and 105, respectively.

The mutation may be, for instance, a point mutation, a substitution (replacing one amino acid by another), addition and/or deletion mutation.

According to specific embodiments, the at least one mutation is a substitution mutation.

According to specific embodiments, the at least one mutation is a single mutation.

According to other specific embodiments, the at least one mutation comprise at least two mutations.

According to other specific embodiments, the at least one mutation comprise at least three or at least four mutations.

According to specific embodiments, the at least one mutation is located within amino acid positions 40-60 of the D1 domain of LILRB.

According to specific embodiments, the at least one mutation is located within amino acid positions 43-45, 47-50 and/or 55-77 of LILRB.

According to specific embodiments, the at least one mutation is in an amino acid selected from the group consisting of S, I, T and V.

According to specific embodiments, the at least one mutation is in an amino acid of the interface between LILRB and Phe195 of the HLA-G wherein the numbering corresponding to SEQ ID NO: 3.

According to specific embodiments, the LILRB2 polypeptide comprises at least one mutation at an amino acid position selected from the group consisting of S45, I49, T50 and V57 corresponding to SEQ ID NO: 1.

As used herein, the phrase “corresponding to SEQ ID NO: 1”, intends to include the corresponding amino acid residue relative to any other LILRB2 amino acid sequence.

According to specific embodiments, the mutation comprises a conservative substitution.

According to other specific embodiments, the mutation comprises a non-conservative substitution.

According to specific embodiments, the mutation is a non-naturally occurring mutation.

According to specific embodiments, the S45 corresponding to SEQ ID NO: 1 comprises a S45R, S45N, S45Q, S45H, S45L, S45K, S45M, S45F, S45W or S45Y mutation.

According to specific embodiments, the S45 corresponding to SEQ ID NO: 1 comprises a S45Q mutation.

According to specific embodiments, the mutation in I49 corresponding to SEQ ID NO: 1 comprises a I49R, I49K, I49F or I49Y mutation.

According to specific embodiments, the mutation in I49 corresponding to SEQ ID NO: 1 comprises a I49K mutation.

According to specific embodiments, the mutation in T50 corresponding to SEQ ID NO: 1 comprises a T50R, T50N, T50L, T50K, T50F, T50W or T50Y mutation,

According to specific embodiments, the mutation in T50 corresponding to SEQ ID NO: 1 comprises a T50F mutation.

According to specific embodiments, the mutation in V57 corresponding to SEQ ID NO: 1 comprises a V57R, V57K, V57F or V57W mutation.

According to specific embodiments, the mutation in V57 corresponding to SEQ ID NO: 1 comprises a V57R mutation.

According to specific embodiments, the LILRB2 polypeptide comprises one of the disclosed mutations.

According to specific embodiments, the LILRB2 polypeptide comprises at least two of the disclosed mutations.

Hence, according to specific embodiments, the LILRB2 polypeptide comprises mutations at amino acids S45 and I49 corresponding to SEQ ID NO: 1, mutations at amino acids S45 and T50 corresponding to SEQ ID NO: 1, mutations at amino acids S45 and V57 corresponding to SEQ ID NO: 1, mutations at amino acids I49 and T50 corresponding to SEQ ID NO: 1, mutations at amino acids I49 and V57 corresponding to SEQ ID NO: 1 or mutations at amino acids T50 and V57 corresponding to SEQ ID NO: 1.

According to specific embodiments, the LILRB2 polypeptide comprises a mutation at amino acid S45 corresponding to SEQ ID NO: 1 and an additional mutation at amino acid I49, T50 and/or V57 corresponding to SEQ ID NO: 1.

According to specific embodiments, the LILRB2 polypeptide comprises S45N and T50R mutations corresponding to SEQ ID NO: 1.

According to specific embodiments, the LILRB2 polypeptide comprises S45Y and T50K mutations corresponding to SEQ ID NO: 1.

According to specific embodiments, the LILRB2 polypeptide comprises S45R and I49F mutations corresponding to SEQ ID NO: 1.

According to specific embodiments, the LILRB2 polypeptide comprises S45Q and V57R mutations corresponding to SEQ ID NO: 1.

According to specific embodiments, the LILRB2 polypeptide comprises S45Q and I49K mutations corresponding to SEQ ID NO: 1.

According to specific embodiments, the LILRB2 polypeptide comprises S45Y and T50N mutations corresponding to SEQ ID NO: 1.

According to specific embodiments, the LILRB2 polypeptide amino acid sequence is at least 70%, at least 75%, at least 80%, at least 81%, at least 82%, at least 83%, at least 84%, at least 85%, at least 86%, at least 87%, at least 88%, at least 89%, at least 90%, at least 91% at least 92%, at least 93%, at least 94%, at least 95%, at least 96%, at least 97%, at least 98%, at least 99% or 100% identical or homologous to an amino acid sequence selected from the group consisting of SEQ ID NO: 5, 7, 9, 11, 13, 15, 17, 19, 21 and 23; or at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identical to the polynucleotide sequence encoding same (as further described hereinbelow).

According to specific embodiments, the LILRB2 polypeptide amino acid sequence is at least 90% identical to an amino acid sequence selected from the group consisting of SEQ ID NO: 5, 7, 9, 11, 13, 15, 17, 19, 21 and 23, each possibility represents a separate embodiments of the present invention.

According to specific embodiments, the LILRB2 polypeptide amino acid sequence comprises an amino acid sequence selected from the group consisting of SEQ ID NO: 5, 7, 9, 11, 13, 15, 17, 19, 21 and 23, each possibility represents a separate embodiments of the present invention.

According to specific embodiments, the LILRB2 polypeptide amino acid sequence is as set forth in SEQ ID NO: 5, 7, 9, 11, 13, 15, 17, 19, 21 or 23, each possibility represents a separate embodiments of the present invention.

According to specific embodiments, the LILRB1 polypeptide comprises at least one mutation at an amino acid position selected from the group consisting of T43, 147, T48 and V55 corresponding to SEQ ID NO: 102.

As used herein, the phrase “corresponding to SEQ ID NO: 102”, intends to include the corresponding amino acid residue relative to any other LILRB1 amino acid sequence.

According to specific embodiments, the mutation comprises a conservative substitution.

According to other specific embodiments, the mutation comprises a non-conservative substitution.

According to specific embodiments, the mutation is a non-naturally occurring mutation.

According to specific embodiments, the T43 corresponding to SEQ ID NO: 102 comprises a T43R, T43N, T43Q, T43H, T43L, T43K, T43M, T43F, T43W or T43Y mutation.

According to specific embodiments, the mutation in 147 corresponding to SEQ ID NO: 102 comprises a 147R, 147K, 147F or 147Y mutation.

According to specific embodiments, the mutation in T48 corresponding to SEQ ID NO: 102 comprises a T48R, T48N, T48L, T48K, T48F, T48W or T48Y mutation.

According to specific embodiments, the mutation in V55 corresponding to SEQ ID NO: 102 comprises a V55R, V55K, V55F or V55W mutation.

According to specific embodiments, the mutation in V55 corresponding to SEQ ID NO: 102 comprises a V55R mutation.

According to specific embodiments, the LILRB1 polypeptide comprises one of the disclosed mutations.

According to specific embodiments, the LILRB1 polypeptide comprises at least two of the disclosed mutations.

According to specific embodiments, the LILRB1 polypeptide amino acid sequence is at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identical or homologous to SEQ ID NO: 103; or at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identical to the polynucleotide sequence encoding same (as further described hereinbelow).

According to specific embodiments, the LILRB1 polypeptide amino acid sequence is at least 90% identical to SEQ ID NO: 103.

According to specific embodiments, the LILRB1 polypeptide amino acid sequence comprises SEQ ID NO: 103.

According to specific embodiments, the LILRB1 polypeptide amino acid sequence is as set forth in SEQ ID NO: 103.

According to specific embodiments, the LILRB (e.g. LILRB2, LILRB1) polypeptide has an increased stability compared to a LILRB (e.g. LILRB2, LILRB1) polypeptide of the same length and sequence not comprising said at least one mutation.

According to specific embodiments, the LILRB2 polypeptide has an increased stability compared to a LILRB2 polypeptide as set forth in SEQ ID NO: 1.

According to specific embodiments, the LILRB1 polypeptide has an increased stability compared to a LILRB2 polypeptide as set forth in SEQ ID NO: 102.

As used herein, the phrase “increased stability” refers to a statistically significant increase in stability of the LILRB (e.g. LILRB2, LILRB1) polypeptide comprising the at least one mutation disclosed herein as compared to LILRB (e.g. LILRB2, LILRB1) polypeptide of the same length and sequence not comprising said at least one mutation. According to specific embodiments, the increased stability is manifested by increased stability of the LILRB-HLAG complex. According to specific embodiments, the increase is in at least 2%, 5%, 10%, 30%, 40% or even higher say, 50%, 60%, 70%, 80%, 90% or 100%. According to specific embodiments, the increase is of at least 1.5 fold, at least 2 fold, at least 3 fold, at least 5 fold, at least 10 fold, or at least 20 fold.

Methods of determining stability of a polypeptide are well known in the art and include e.g. Size-exclusion-high performance liquid chromatography (SEC-HPLC) to defined physical conditions and time dependency of aggregates formation, SDS-PAGE to defined physical conditions and time dependency of the proteins-integrity, analyzing the melting temperature (Tm) e.g. with differential scanning calorimetry (DSC).

According to specific embodiments, the LILRB (e.g. LILRB2, LILRB1) polypeptide has an increased affinity to HLA-G compared to a LILRB (e.g. LILRB2, LILRB1) polypeptide of the same length and sequence not comprising said at least one mutation.

According to specific embodiments, the LILRB2 polypeptide has an increased affinity to HLA-G compared to a LILRB2 polypeptide as set forth in SEQ ID NO: 1.

According to specific embodiments, the LILRB1 polypeptide has an increased affinity to HLA-G compared to a LILRB2 polypeptide as set forth in SEQ ID NO: 102.

As used herein, the phrase “increased affinity to HLA-G” refers to a statistically significant increase in binding affinity of the LILRB (e.g. LILRB2, LILRB1) polypeptide comprising the at least one mutation disclosed herein to an HLA-G polypeptide (such as set forth in SEQ ID NO: 3) as compared to LILRB (e.g. LILRB2, LILRB1) polypeptide of the same length and sequence not comprising said at least one mutation, which may be determined directly or through inhibition of binding of native ligands. The increase in binding affinity may be manifested by a higher affinity (e.g. Kd, Ka) to HLA-G and/or a higher selective binding to HLA-G as compared to other HLAs (e.g. HLA-A, HLA-B, HLA-C). According to specific embodiments, the increase is in at least 2%, 5%, 10%, 30%, 40% or even higher say, 50%, 60%, 70%, 80%, 90% or 100%. According to specific embodiments, the increase is of at least 1.5 fold, at least 2 fold, at least 3 fold, at least 5 fold, at least 10 fold, or at least 20 fold. According to specific embodiments, the increase is of at least 5, 10, 100, 1000 or 10000 fold.

Methods of determining affinity are well known in the art and are also described hereinabove and below and include e.g. BiaCore, HPLC, Surface Plasmon Resonance assay (SPR) and flow cytometry (FACS).

According to specific embodiments, the LILRB (e.g. LILRB2, LILRB1) polypeptide can induce or enhance phagocytosis of e.g. cancer cells.

Methods of determining phagocytosis are known in the art and are further described in the Examples section which follows.

According to specific embodiments, the LILRB (e.g. LILRB2, LILRB1) polypeptide can prevent or reduce induction of tumor-supportive M2 macrophages and lead to induction of tumor-suppressive M1 macrophages.

According to specific embodiments, the LILRB (e.g. LILRB2, LILRB1) polypeptide can convert M0 (M2-like) macrophages to M1 macrophages.

Methods of determining macrophage phenotype are known in the art and are further described in the Examples section which follows.

The LILRB (e.g. LILRB2, LILRB1) polypeptides of some embodiments of the invention may be attached to a non-proteinaceous or a proteinaceous moiety.

Thus, according to an aspect of the present invention, there is provided a composition of matter comprising the LILRB (e.g. LILRB2, LILRB1) polypeptide disclosed herein and a non-proteinaceous moiety attached to the LILRB (e.g. LILRB2, LILRB1) polypeptide.

The phrase “non-proteinaceous moiety” as used herein refers to a molecule not including peptide bonded amino acids that is attached to the peptide. According to a specific embodiment the non-proteinaceous is a non-toxic moiety. Exemplary non-proteinaceous moieties which may be used according to the present teachings include, but are not limited to a drug, a chemical, a small molecule, a polynucleotide, a detectable moiety, polyethylene glycol (PEG), Polyvinyl pyrrolidone (PVP), poly (styrene comaleic anhydride) (SMA), and divinyl ether and maleic anhydride copolymer (DIVEMA). According to specific embodiments of the invention, the non-proteinaceous moiety comprises polyethylene glycol (PEG).

Such a molecule is highly stable (resistant to in-vivo proteolytic activity probably due to steric hindrance conferred by the non-proteinaceous moiety) and may be produced using common solid phase synthesis methods which are inexpensive and highly efficient, as further described hereinbelow. However, it will be appreciated that recombinant techniques may still be used, whereby the recombinant peptide product is subjected to in-vitro modification (e.g., PEGylation as further described hereinbelow).

Bioconjugation of the polypeptide amino acid sequence with PEG (i.e., PEGylation) can be effected using PEG derivatives such as N-hydroxysuccinimide (NHS) esters of PEG carboxylic acids, monomethoxyPEG₂-NHS, succinimidyl ester of carboxymethylated PEG (SCM-PEG), benzotriazole carbonate derivatives of PEG, glycidyl ethers of PEG, PEG p-nitrophenyl carbonates (PEG-NPC, such as methoxy PEG-NPC), PEG aldehydes, PEG-orthopyridyl-disulfide, carbonyldimidazol-activated PEGs, PEG-thiol, PEG-maleimide. Such PEG derivatives are commercially available at various molecular weights [See, e.g., Catalog, Polyethylene Glycol and Derivatives, 2000 (Shearwater Polymers, Inc., Huntsvlle, Ala.)]. If desired, many of the above derivatives are available in a monofunctional monomethoxyPEG (mPEG) form. In general, the PEG added to the polypeptide of the present invention should range from a molecular weight (MW) of several hundred Daltons to about 100 kDa (e.g., between 3-30 kDa). Larger MW PEG may be used but may result in some loss of yield of PEGylated polypeptides. The purity of larger PEG molecules should be also watched, as it may be difficult to obtain larger MW PEG of purity as high as that obtainable for lower MW PEG. It is preferable to use PEG of at least 85% purity, and more preferably of at least 90% purity, 95% purity, or higher. PEGylation of molecules is further discussed in, e.g., Hermanson, Bioconjugate Techniques, Academic Press San Diego, Calif. (1996), at Chapter 15 and in Zalipsky et al., “Succinimidyl Carbonates of Polyethylene Glycol,” in Dunn and Ottenbrite, eds., Polymeric Drugs and Drug Delivery Systems, American Chemical Society, Washington, D.C. (1991).

Conveniently, PEG can be attached to a chosen position in the polypeptide by site-specific mutagenesis as long as the activity of the conjugate is retained. A target for PEGylation could be any Cysteine residue at the N-terminus or the C-terminus of the peptide sequence. Additionally or alternatively, other Cysteine residues can be added to the polypeptide amino acid sequence (e.g., at the N-terminus or the C-terminus) to thereby serve as a target for PEGylation. Computational analysis may be effected to select a preferred position for mutagenesis without compromising the activity.

Various conjugation chemistries of activated PEG such as PEG-maleimide, PEG-vinylsulfone (VS), PEG-acrylate (AC), PEG-orthopyridyl disulfide can be employed. Methods of preparing activated PEG molecules are known in the arts. For example, PEG-VS can be prepared under argon by reacting a dichloromethane (DCM) solution of the PEG-OH with NaH and then with di-vinylsulfone (molar ratios: OH 1:NaH 5:divinyl sulfone 50, at 0.2 gram PEG/mL DCM). PEG-AC is made under argon by reacting a DCM solution of the PEG-OH with acryloyl chloride and triethylamine (molar ratios: OH 1:acryloyl chloride 1.5:triethylamine 2, at 0.2 gram PEG/mL DCM). Such chemical groups can be attached to linearized, 2-arm, 4-arm, or 8-arm PEG molecules.

Resultant conjugated molecules (e.g., PEGylated or PVP-conjugated polypeptide) are separated, purified and qualified using e.g., high-performance liquid chromatography (HPLC) as well as biological assays.

According to specific embodiments, the non-proteinaceous moiety is a dimerizing moiety. Such dimerizing moieties are well known to the skilled in the art and are further described hereinbelow.

According to an additional or an alternative aspect of the present invention there is provided a fusion polypeptide comprising the LILRB (e.g. LILRB2, LILRB1) polypeptide disclosed herein attached to a heterologous proteinaceous moiety.

As used herein, the term “fusion polypeptide” refers to an amino acid sequence having two or more parts which are not found together in a single amino acid sequence in nature.

As used herein, the term “heterologous” refers to an amino acid sequence which is not native to the recited amino acid sequence (e.g. a LILRB polypeptide e.g. a LILRB2 polypeptide, a LILRB1 polypeptide) at least in localization or is completely absent from the native sequence of the recited amino acid sequence.

Non-limiting examples of heterologous proteinaceous moieties that can be fused to the LILRB (e.g. LILRB2, LILRB1) polypeptide of some embodiments of the invention include, a dimerizing moiety, a detectable moiety, a therapeutic moiety, a cleavable moiety and the like, as further described hereinbelow.

According to specific embodiments, the heterologous proteinaceous moiety is a dimerizing moiety. Such dimerizing moieties are well known to the skilled in the art and are further described hereinbelow.

As used herein the term “dimerizing moiety” refers to a moiety capable of attaching two different monomers to form a dimer. Such dimerizing moieties are known in the art and include chemical and proteinaceous moieties.

According to specific embodiments, the dimerizing moiety is directly attached to the polypeptide.

According to specific embodiments, the dimerizing moiety is non-directly attached to the polypeptide.

According to specific embodiments, the dimerizing moiety is covalently attached to the polypeptide.

According to specific embodiments, the dimerizing moiety is non-covalently attached to the polypeptide.

According to specific embodiments, the dimerizing moiety is a composition of at least two different molecules.

According to specific embodiments, the dimerizing moiety is a non-proteinaceous moiety, e.g. a cross linker, an organic polymer, a synthetic polymer, a small molecule and the like.

Numerous such non-proteinaceous moieties are known in the art and can be commercially obtained from e.g. Santa Cruz, Sigma-Aldrich, Proteochem and the like. According to specific embodiments, the non-proteinaceous moiety is a heterobifunctional cross linker. Heterobifunctional cross linkers have two different reactive ends. Typically, in the first step, a monomer is modified with one reactive group of the heterobifunctional reagent; the remaining free reagent is removed. In the second step, the modified monomer is mixed with a second monomer, which is then allowed to react with modifier group at the other end of the reagent. The most widely used couple proteins through amine and sulfhydryl groups (the least stable amine reactive NHS-esters couple first and after removal of uncoupled reagent, the coupling to the sulfhydryl group proceeds). The sulfhydryl reactive groups are generally maleimides, pyridyl disulfides and alpha-halocetyls. Other crosslinkers include carbodiimides, which link between carboxyl groups (—COOH) and primary amines (—NH2). Another approach is to modify the lysine residues of one monomer to thiols and the second monomer is modified by addition of maleimide groups followed by formation of stable thioester bonds between the monomers. If one of the monomers has native thiols, these groups can be reacted directly with maleimide attached to the other monomer. There are also heterobifunational cross-linkers with one phororeactive end, such as Bis [2-(4-azidosalicylamido)ethyl)] disulfide, BASED. Photoreactive groups are used when no specific groups are available to react with—as photoreactive groups react non-specifically upon exposure to UV light. Non-limiting Examples of such heterobifunctional cross linkers include, but are not limited to: Alkyne-PEG4-malcimide, Alkyne-PEG5-N-hydroxysuccinimidyl ester, Maleimide-PEG-succinimidyl ester, Azido-PEG4-phenyloxadiazole methylsulfone, LC-SMCC (succinimidyl-4-(N-malcimidomethyl)cyclohexane-1-carboxy-(6-amidocaproate)), MPBH (4-(4-N-Malcimidophenyl) butyric acid hydrazide hydrochloride+1/2 dioxane), PDPH (3-(2-pyridyldithio) propionyl hydrazide), SIAB (N-succinimidyl (4-iodoacetyl)aminobenzoate), SMPH (succinimidyl-6-((b-malcimidopropionamido) hexanoate), Sulfo-KMUS (N—(K-malcimidoundecanoyloxy) sulfosuccinimide ester), Sulfo-SIAB (sulfosuccinimidyl (4-iodoacetyl)aminobenzoate), 3-(Maleimido) propionic acid N-hydroxysuccinimide ester, Methoxycarbonylsulfenyl chloride, ester, BocNH-PEG5-acid, BMPH (N—(B-Propargyl-PEG-acid, Amino-PEG-t-butyl maleimidopropionic acid) hydrazide, trifluoroacetic acid salt), ANB-NOS, BMPS, EMCS, GMBS, LC-SPDP, MBS, SBA, SIA, Sulfo-SIA, SMCC, SMPB, SMPH, SPDP, Sulfo-LC-SPDP, Sulfo-MBS, Sulfo-SANPAH, Sulfo-SMCC.

According to other specific embodiments, the dimerizing moiety is a proteinaceous moiety.

According to specific embodiments, the LILRB (e.g. LILRB2, LILRB1) polypeptide is attached to an N-terminus of the dimerizing proteinaceous moiety.

According to specific embodiments, the LILRB (e.g. LILRB2, LILRB1) polypeptide is attached to a C-terminus of the dimerizing proteinaceous moiety.

According to specific embodiments, the dimerizing moiety comprises members of affinity pair polypeptides having two distinct affinity moieties for two different affinity complementary tags. Such affinity pairs are well known in the art and include, but are not limited to hemagglutinin (HA), anti-HA, AviTagTM, V5, Myc, T7, FLAG, HSV, VSV-G, His, biotin, avidin, streptavidin, rhizavedin, metal affinity tags, lectins affinity tags. The skilled artisan would know which tag to select.

According to specific embodiments, the dimerizing moiety comprises a leucine zipper or a helix-loop-helix.

According to specific embodiments, the dimerizing moiety is an Fc domain of an antibody or a fragment thereof.

According to specific embodiments, the Fc is of IgG, IgA, IgD or IgE.

According to specific embodiments, the Fc domain of IgG.

According to specific embodiments, the Fc domain is of IgG1 or IgG4.

According to specific embodiments, the Fc domain is of human IgG4.

A non-limiting example of human IgG4 Fc domain that can be used with specific embodiments of the invention is provided in SEQ ID NO: 63.

According to specific embodiments, the Fc domain is of human IgG1.

A non-limiting example of human IgG1 Fc domain that can be used with specific embodiments of the invention is provided in SEQ ID NO: 64.

According to specific embodiments, the Fc domain may comprise conservative and non-conservative amino acid substitutions (detailed description on conservative and non-conservative substitutions is provided hereinbelow). Such substitutions in an Fc domain are known in the art and are further described hereinbelow.

For example, there are a number of mechanisms that can be used to generate a heterodimer using an Fc domain of an antibody, such as, but not limited to, knob-into-hole or charge pairs (see e.g. Gunasekaran et al., J. Biol. Chem. (2010) 285 (25): 19637, hereby incorporated by reference in its entirety).

A representative example, which can be used with specific embodiments of the invention is the “knob-into-hole” (“KIH”) form. Such knob and hole mutations are well known in the art and disclosed e.g. in U.S. Pat. No. 8,216,805, Shane Atwell et Al. J. Mol. Biol. (1997) 270, 26-35; Cater et al. (Protein Engineering vol. 9 no. 7 pp. 617-621, 1996); and A. Margaret Merchant et. al. Nature Biotechnology (1998) 16 July, the contents of which are fully incorporated herein by reference. In addition, as described in Merchant et al., Nature Biotech. 16:677 (1998), these “knobs and hole” mutations can be combined with disulfide bonds to skew formation to heterodimerization.

Thus, according to specific embodiments, one of the monomers comprises an Fc domain comprising a knob mutation(s) and the other monomer comprises an Fc domain comprising a hole mutation(s).

It is within the scope of those skilled in the art to select a specific immunoglobulin Fc domain from particular immunoglobulin classes and subclasses and to select a first Fc variant for knob mutation and the other for hole mutation. Non-limiting Examples of substitutions that can be used with specific embodiments include S228P, L235E, T366W, Y349C, T366S, L368A, Y407V and/or E356C [according to EU numbering (Kabat, E. A., T. T. Wu, M. Reid-Miller, H. M. Perry and K. S. Gottesman. 1987. Sequences of proteins of Immunological Interest. US. Dept. of Health and Human Services, Bethesda), corresponding to the human IgG4 as part of a full length antibody], or L235A, Y349C, T366W, T354C, D356C, T366S, L368A and/or Y407V [according to EU numbering (Kabat, E. A., T. T. Wu, M. Reid-Miller, H. M. Perry and K. S. Gottesman. 1987. Sequences of proteins of Immunological Interest. US. Dept. of Health and Human Services, Bethesda) corresponding to the human IgG1 as part of a full length antibody].

Non-limiting examples of IgG4 Fc domains comprising a knob mutation that can be used with specific embodiments of the invention are provided in SEQ ID NO: 65, 59 and 60.

Non-limiting examples of IgG4 Fc domains comprising a hole mutation that can be used with specific embodiments of the invention are provided in SEQ ID NO: 66, 61 and 62.

Non-limiting examples of IgG1 Fc domains comprising a knob mutation that can be used with specific embodiments of the invention are provided in SEQ ID NO: 31, 67 and 86.

Non-limiting example of IgG1 Fc domains comprising a hole mutation that can be used with specific embodiments of the invention is provided in SEQ ID NO: 29, 68 and 85.

According to specific embodiments, the Fc domain comprises an amino acid sequence having at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identity or homology to an amino acid sequence selected from the group consisting of SEQ ID NO: 63, 64, 65, 59, 60, 66, 61, 62, 31, 67, 86, 29, 68 and 85 or a functional fragment thereof which exhibits the desired activity as disclosed herein; or at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identity to the polynucleotide sequence encoding same.

According to specific embodiments, the Fc domain is modified to alter it's binding to an Fc receptor, reduce an immune activating function thereof and/or improve half-life of the fusion.

According to other specific embodiments, the Fc domain is not-modified to alter it's binding to an Fc receptor, reduce an immune activating function thereof and/or improve half-life of the fusion.

According to specific embodiments, the Fc domain is modified to reduce or prevent binding to Fc receptors (e.g. Fc.gamma.RI, Fc.gamma.RII and Fc.gamma.RIII) in vivo. Such modifications have been described by, for example, Clark and colleagues, who have designed and described a series of mutant IgG1, IgG2 and IgG4 Fc domains and their Fc.gamma.R binding properties (Armour et al., 1999; Armour et al., 2002, the content of which are incorporated herein by reference in their entirety). Additional or alternative modifications in the Fc of human IgG1 to reduce it binding to Fc receptors are described by CHAPPEL and colleagues (Proc. Natl. Acad. Sci (1991) 88:9036-9040, the content of which are incorporated herein by reference in their entirety), who identified amino acids L234 and L235 [according to EU numbering (Kabat et al.) corresponding to a full length antibody] as essential for Fc receptor binding. An additional substitution of P329 to G even weaker the binding, this LALA-PG combination of substitutions is described by e.g. Schlothauer, T., et al. (2016) Protein Eng. Des. Sel. 29, 457-466; and International Patent Application Publication No. WO 2012/130831, the contents of which are incorporated herein by reference in their entirety). Additional or alternative modifications in the Fc of human IgG4 to prevent Fab arm exchange and to reduce it binding to Fc receptor are described by John-Paul Silva et al., (THE JOURNAL OF BIOLOGICAL CHEMISTRY (2015), 290:9, 5462-5469, the content of which are incorporated herein by reference in their entirety) and Newman et al., (Clinical Immunology (2001) 98:2, the content of which are incorporated herein by reference in their entirety), who identified S228P and L235E [according to EU numbering (Kabat et al.) corresponding to a full length antibody], respectively.

According to specific embodiments, the Fc domain is modified to maximize FcγRIIIa binding. Such modifications have been described by, for example, Shields R L J Biol Chem. (2001) 276:6591, Smith P, Proc Natl Acad Sci USA. (2012) 109:6181, Stavenhagen et al., Cancer Res (2007) 67:8882, Lazar et al., Proc Natl Acad Sci UCA (2006) 103:4005, Richards et al., 2008 Cancer Ther 7:2517 and Mimoto et al., (2013) MAbs 5:229, the content of which are incorporated herein by reference in their entirety. Non-limiting examples of such modifications which can be used with specific embodiments include substitution in one or more amino residues [according to EU numbering (Kabat et al.) corresponding to a full length antibody] selected from S298, E333 and K334 (e.g. S298A, E333A, K334A); G236A, S239A, A330L and 1332E; F243L, R292P, Y300L, V3051 and P396L; S239D, 1332E and A330L; 236A, S239D and 1332E; and asymmetric substitution-L234Y/L235Q/G236W/S239M/H268D/D270E/S298A in one heavy chain and D270E/K326D/A330M/K334E in the opposing heavy chain.

According to specific embodiments, the Fc domain is modified to alter effector function, such as to reduce complement binding and/or to reduce or abolish complement dependent cytotoxicity. Such modifications have been described in, for example, U.S. Pat. Nos. 5,624,821 and 5,648,260, 6,194,551, WO 99/51642, Wines et al., 2000, Idusogie et al. (2000) J. Immunol. 164:4178; Tao et al. (1993) J. Exp. Med. 178:661 and Canfield & Morrison (1991) J. Exp. Med. 173:1483, the content of which are incorporated herein by reference in their entirety. Non-limiting examples of such modifications which can be used with specific embodiments include substitution in one or more amino acids at positions [according to EU numbering (Kabat et al.) corresponding to a full length antibody] selected from 234, 235, 236, 237, 297, 318, 320 and 322; 329, 331 and 322; L234 and/or L235 (e.g. L234A and/or L235A); D270, K322, P329 and P331 (e.g. D270A, K322A, P329A and P331A).

According to specific embodiments, Fc domain is modified to improve the half-life of the fusion protein. Such alterations are described for instance in U.S. Pat. Nos. 5,869,046 and 6,121,022, the content of which are incorporated herein by reference in their entirety. For example, substitution in one or more amino acids at positions [according to EU numbering (Kabat et al.) corresponding to a full length antibody] selected from 252 (e.g., to introduce Thr), 254 (e.g., to introduce Ser) and 256 (e.g., to introduce Phe). Another modification to improve half-life may be by altering the CH1 or CL region to introduce a salvage receptor motif, such as that found in the two loops of a CH2 domain of an Fc region of an IgG.

Maximizing FcRn binding and extending half-life has also been described e.g. in Stapleton N M, Nat Commun. (2011) 2:599, Shields R L. J Biol Chem. (2001) 276:6591, Dall'acqua WF J Immunol. (2002) 169:5171, Zalevsky J, Nat Biotechnol. (2010) 28:157, Ghetie V, Nat. Biotechnol. (1997) 15:637 and Monnet C, MAbs. (2014) 6:422, the content of which are incorporated herein by reference in their entirety. Non-limiting examples of such modifications which can be used with specific embodiments include substitution in one or more amino acids residues [according to EU numbering (Kabat et al.) corresponding to a full length antibody] selected from Arg435His; Asn434Ala; Met252Tyr, Ser254Thr, and Thr256Glu; Met428Leu and Asn434Ser; Thr252Leu, Thr253Ser and Thr254Phe; Glu294delta, Thr307Pro and Asn434Tyr; Thr256Asn, Ala378Val, Ser383Asn and Asn434Tyr.

Non-limiting examples of LILRB2-Fc fusion sequences which may be used with specific embodiments of the invention are described in Table 3 hereinbelow.

Non-limiting examples of LILRB2-Fc fusion sequences that can be used with specific embodiments of the invention are provided in SEQ ID NO: 37, 39, 41, 43, 45, 47, 49, 51, 53, 55, 57, 96, 98, and 101.

According to specific embodiments, the LILRB2-Fc fusion comprises an amino acid sequence having at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identity or homology to an amino acid sequence selected from the group consisting of SEQ ID NO: 39, 41, 43, 45, 47, 49, 51, 53, 55 and 57; or at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identity to the polynucleotide sequence encoding same (as further described hereinbelow).

According to specific embodiments, the LILRB2-Fc fusion amino acid sequence is at least 90% identical to an amino acid sequence selected from the group consisting of SEQ ID NO: 39, 41, 43, 45, 47, 49, 51, 53, 55 and 57, each possibility represents a separate embodiments of the present invention.

According to specific embodiments, the LILRB2-Fc fusion amino acid sequence comprises an amino acid sequence selected from the group consisting of SEQ ID NO: 39, 41, 43, 45, 47, 49, 51, 53, 55 and 57, each possibility represents a separate embodiments of the present invention.

According to specific embodiments, the LILRB2-Fc fusion amino acid sequence is as set forth in SEQ ID NO: 39, 41, 43, 45, 47, 49, 51, 53, 55 and 57, each possibility represents a separate embodiments of the present invention.

As the LILRB (e.g. LILRB2, LILRB1) polypeptide of some embodiments comprises a dimerizing moiety, according to an additional or an alternative aspect of the present invention there is provided a dimer comprising the LILRB (e.g. LILRB2, LILRB1) polypeptide, the composition comprising same or the fusion polypeptide comprising same disclosed herein.

According to an additional or an alternative aspect of the present invention there is provided a composition comprising the dimer disclosed herein, wherein the dimer is the predominant form of the LILRB (e.g. LILRB2, LILRB1) in the composition.

According to specific embodiments at least 55%, at least 60%, at least 65%, at least 70%, at least 75%, at least 80%, at least 85%, at least 90%, at least 95%, at least 96%, at least 97%, at least 98%, at least 99% of the LILRB2 polypeptide in the composition is the dimer disclosed herein.

According to specific embodiments at least 90% of the LILRB (e.g. LILRB2, LILRB1) polypeptide in the composition is the dimer disclosed herein.

Methods of determining dimerization are well known in the art and include, but are not limited to NATIVE-PAGE, SEC-HPLC 2D gels, gel filtration, SEC-MALS, Analytical ultracentrifugation (AUC) Mass spectrometry (MS), capillary gel electrophoresis (CGE).

According to specific embodiments, the monomers of the dimer are not covalently attached.

According to other specific embodiments, the monomers of the dimer are covalently attached.

According to other specific embodiments, the monomers of the dimer are attached by a disulfide bond.

The dimer may be a homodimer or a heterodimer.

According to specific embodiments, the dimer is a heterodimer.

As used herein, the term “heterodimer” refers to a non-naturally occurring dimeric protein formed by the artificial attachment of two different proteins (referred to herein as monomers).

Thus, according to an additional or an alternative aspect of the present invention there is provided a heterodimer comprising a first monomer comprising the LILRB (e.g. LILRB2, LILRB1) polypeptide, the composition comprising same or the fusion polypeptide comprising same disclosed herein, and a second monomer comprising a polypeptide distinct from said LILRB (e.g. LILRB2, LILRB1).

It is within the scope of those skilled in the art to select the suitable distinct polypeptide comprised in the second monomer. Non-limiting examples of such polypeptides include a type I membrane protein, a type II membrane protein and an immune check point protein or functional fragments or homologs thereof capable of at least binding their natural binding pair.

As used herein, the phrase “type I membrane protein” refers to a transmembrane protein having an N-terminus extracellular domain.

Non-limiting examples of such Type I membrane proteins which can be used with specific embodiments of the invention include PD1, SIRPα, LAG3, BTN3A1, CD27, CD80, CD86, ENG, NLGN4X, CD84, TIGIT, CD40, IL-8, IL-10, CD164, LY6G6F, CD28, CTLA4, BTLA, LILRB1, LILRB4, TYROBP, ICOS, VEGFA, CSF1, CSFIR, VEGFB, BMP2, BMP3, GDNF, PDGFC, PDGFD, RAETIE, CD155, CD166, MICA, NRG1, HVEM, DR3, TEK, TGFBR (e.g. TGFBR1), LY96, CD96, KIT, CD244 GFER and SIGLEC (e.g. SIGLEC10).

According to specific embodiments, the type I membrane protein is selected from the group consisting of PD1, SIRPα, LAG3, TIGIT, LILRB1, CSF1, CSFIR and TGFBR.

According to specific embodiments, the type I membrane protein is selected from the group consisting of PD1, SIRPα, TIGIT and SIGLEC.

According to specific embodiments, the Type I membrane protein is an immune modulator.

As used herein the term “immune modulator” refers to a protein that modulates an immune cell response (i.e. activation or function). Immune modulators can positively regulate immune cell activation or function or negatively regulate immune cell activation or function. Such immune modulators are known in the art and include an immune-check point protein, a cytokine and the like.

According to specific embodiments, the immune modulator is an immune activator.

According to other specific embodiments, the immune modulator is an immune suppressor or inhibitor.

Non-limiting examples of Type I membrane protein immune modulators include, but are not limited to PD1, SIRPα, CD28, CSFIR, IL-8, IL-10, CTLA4, ICOS, CD27, CD80, CD86, SIGLEC10 and TIGIT.

As used herein, the phrase “type II membrane protein” refers to a transmembrane protein having a C-terminus extracellular domain.

Non-limiting examples of such Type II membrane proteins that can be used with specific embodiments of the invention include 4-1BBL, FasL, TRAIL, TNF-alpha, TNF-beta, OX40L, CD40L, CD27L, CD30L, RANKL, TWEAK, APRIL, BAFF, LIGHT, VEGI, GITRL, EDAI/2, Lymphotoxin alpha and Lymphotoxin beta.

According to specific embodiments, the type II membrane protein is selected from the group consisting of 4-1BBL, OX40L, CD40L, LIGHT and GITRL.

According to specific embodiments, the Type II membrane protein is an immune modulator.

Such immune modulators include, but are not limited to 4-1BBL, TNF-alpha, TNF-beta, OX40L, CD40L, CD27L and CD30L.

As used herein the term “immune-check point protein” refers to a protein that regulates an immune cell activation or function. Immune check-point proteins can be either co-stimulatory proteins (i.e. transmitting a stimulatory signal resulting in activation of an immune cell) or inhibitory proteins (i.e. transmitting an inhibitory signal resulting in suppressing activity of an immune cell). According to some embodiments, the immune check-point protein regulates activation or function of a T cell. Numerous checkpoint proteins are known in the art and include, but not limited to, PD1, PDL-1, B7H2, B7H4, CTLA-4, CD80, CD86, LAG-3, TIM-3, KIR, IDO, CD19, OX40, 4-1BB (CD137), CD27, CD70, CD40, GITR, CD28 and ICOS (CD278).

According to specific embodiments, a first monomer of the heterodimer comprises the LILRB (e.g. LILRB2, LILRB1) polypeptide disclosed herein and a second monomer comprising an amino acid sequence of a protein selected from the group consisting of SIRPα, PD1, TIGIT and SIGLEC10, wherein said amino acid sequence is capable of binding its natural binding pair.

According to specific embodiments, a first monomer of the heterodimer comprises the LILRB (e.g. LILRB2, LILRB1) polypeptide disclosed herein and a second monomer comprising an amino acid sequence of SIRPα, wherein said amino acid sequence is capable of binding CD47.

Non-limiting examples of LILRB2 and SIRPα heterodimers sequences which may be used with specific embodiments of the invention are described in Table 3 hereinbelow.

“SIRPα (Signal Regulatory Protein Alpha, also known as CD172a)” refers to the polypeptide encoded by the SIRPA gene (Gene ID 140885). According to specific embodiments, SIRPα is human SIRPα. According to a specific embodiment, the SIRPα refers to the human SIRPα, such as provided in the following GenBank Number NP_001035111, NP_001035112, NP_001317657 or NP_542970.

The known binding pair of SIRPα is CD47. According to a specific embodiment, the CD47 protein refers to the human protein, such as provided in the following GenBank Numbers NP_001768 or NP_942088.

As used herein, the term “SIRPα polypeptide” refers to full-length SIRPα, functional fragments thereof or homologs thereof which maintain at least the ability to bind CD47. For example, according to specific embodiments, the amino acid sequences of SIRPα comprise substitution, addition and deletion mutations as further described hereinabove and below.

According to specific embodiments, the SIRPα polypeptide comprises an extracellular domain of the SIRPα or a functional fragment thereof capable of binding CD47.

According to specific embodiments, the SIRPα polypeptide comprises SEQ ID NO: 27 or a functional fragment thereof capable of binding CD47.

According to specific embodiments, the SIRPα polypeptide comprises SEQ ID NO: 27.

According to specific embodiments, the SIRPα polypeptide consists of SEQ ID NO: 27.

According to specific embodiments, SIRPα polypeptide comprises SEQ ID NO: 88 or a functional fragment thereof capable of binding CD47.

According to specific embodiments, SIRPα polypeptide comprises SEQ ID NO: 88.

According to specific embodiments, SIRPα polypeptide consists of SEQ ID NO: 88.

The term “SIRPα polypeptide” also encompasses functional homologues which exhibit the desired activity (i.e., binding CD47). Such homologues can be, for example, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identical or homologous to the SIRPα sequences that are described herein (e.g. SEQ ID NO: 27, 88); or at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identical to the polynucleotide sequence encoding same (as further described hereinbelow).

According to specific embodiments, the SIRPα amino acid sequence is at least 90% identical to SEQ ID NO: 27 or 88.

According to specific embodiments, the SIRPα polypeptide may comprise conservative and non-conservative amino acid substitutions (detailed description on conservative and non-conservative substitutions is provided hereinbelow). Non-limiting examples of such substitutions are known in the art and disclosed e.g. in Weiskopf K et al. Science. (2013); 341 (6141): 88-91, the contents of which are fully incorporated herein by reference.

According to specific embodiments, SIRPα polypeptide comprises 100-504, 100-500 amino acids, 150-450 amino acids, 200-400 amino acids, 250-400 amino acids, 300-400 amino acids, 320-420 amino acids, 340-350 amino acids, 300-400 amino acids, 340-450 amino acids, 100-200 amino acids, 100-150 amino acids, 100-125 amino acids, 100-120 amino acids, 100-119 amino acids, 105-119 amino acids, 110-119 amino acids, 115-119 amino acids, 105-118 amino acids, 110-118 amino acids, 115-118 amino acids, 105-117 amino acids, 110-117 amino acids, 115-117 amino acids, each possibility represents a separate embodiment of the present invention.

According to a specific embodiment, a first monomer of the heterodimer comprises a LILRB2 polypeptide comprising an amino acid sequence having at least at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identity to an amino acid sequence selected from the group consisting of SEQ ID NO: 5, 7, 9, 11, 13, 15, 17, 19, 21 and 23 and a second monomer comprising an amino acid sequence of SIRPα, wherein said amino acid sequence comprises an amino acid sequence having at least at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identity to SEQ ID NO: 27 or 88.

According to a specific embodiment, a first monomer of the heterodimer comprises a LILRB2 polypeptide comprising an amino acid sequence selected from the group consisting of SEQ ID NO: 5, 7, 9, 11, 13, 15, 17, 19, 21 and 23 and a second monomer comprising an amino acid sequence of SIRPα, wherein said amino acid sequence comprises SEQ ID NO: 27 or 88.

According to a specific embodiment, a first monomer of the heterodimer comprises an amino acid sequence having at least at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identity to an amino acid sequence selected from the group consisting of SEQ ID NO: 39, 41, 43, 45, 47, 49, 51, 53, 55 and 57 and a second monomer of the heterodimer comprises an amino acid sequence having at least at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identity to SEQ ID NO: 35, 90, 94, and 98.

According to a specific embodiment, a first monomer of the heterodimer comprises an amino acid sequence selected from the group consisting of SEQ ID NO: 39, 41, 43, 45, 47, 49, 51, 53, 55 and 57 and a second monomer of the heterodimer comprises SEQ ID NO: 35.

According to a specific embodiment, a first monomer of the heterodimer is as set forth in SEQ ID NO: 39, 41, 43, 45, 47, 49, 51, 53, 55 or 57 and a second monomer of the heterodimer is as set forth in SEQ ID NO: 35, 90, 94 or 98.

According to specific embodiments, the heterodimer comprising the LILRB (e.g. LILRB2, LILRB1) polypeptide disclosed herein and the amino acid sequence of SIRPα can induce or enhance phagocytosis of e.g. cancer cells.

According to specific embodiments, the heterodimer comprising the LILRB (e.g. LILRB2, LILRB1) polypeptide disclosed herein and the amino acid sequence of SIRPα can prevent or reduce induction of tumor-supportive M2 macrophages and lead to induction of tumor-suppressive M1 macrophages.

According to specific embodiments, the heterodimer comprising the LILRB (e.g. LILRB2, LILRB1) polypeptide disclosed herein and the amino acid sequence of SIRPα can convert M0 (M2-like) macrophages to M1 macrophages.

According to specific embodiments, heterodimer comprising the LILRB (e.g. LILRB2, LILRB1) polypeptide disclosed herein and the amino acid sequence of SIRPα binds cells expressing both CD47 and HLA-G and does not significantly bind cells expressing only one of CD47 and HLA-G e.g. as determined by flow cytometry analysis.

According to specific embodiments, the heterodimer comprising the LILRB (e.g. LILRB2, LILRB1) polypeptide disclosed herein and the amino acid sequence of SIRPα does not significantly bind red blood cells (RBCs) e.g. as determined by flow cytometry analysis.

According to specific embodiments, the LILRB (e.g. LILRB2, LILRB1) polypeptide or composition, fusion or dimer comprising same is attached to or comprises a heterologous therapeutic moiety. The therapeutic moiety may be any molecule, including small molecule chemical compounds and polypeptides.

Non-limiting examples of therapeutic moieties which can be used with specific embodiments of the invention include a cytotoxic moiety, a toxic moiety, a cytokine moiety, an immunomodulatory moiety (e.g. an immune check point moiety, a cytokine, as further described hereinabove), a polypeptide, an antibody, a drug, a chemical and/or a radioisotope.

According to some embodiments of the invention, the therapeutic moiety is conjugated by translationally fusing the polynucleotide encoding the polypeptide of some embodiments of the invention with the nucleic acid sequence encoding the therapeutic moiety.

Additionally or alternatively, the therapeutic moiety can be chemically conjugated (coupled) to the LILRB (e.g. LILRB2, LILRB1) polypeptide or composition, fusion or dimer comprising same of some embodiments of the invention, using any conjugation method known to one skilled in the art. For example, a peptide can be conjugated to an agent of interest, using a 3-(2-pyridyldithio) propionic acid Nhydroxysuccinimide ester (also called N-succinimidyl 3-(2-pyridyldithio) propionate) (“SDPD”) (Sigma, Cat. No. P-3415; see e.g., Cumber et al. 1985, Methods of Enzymology 112:207-224), a glutaraldehyde conjugation procedure (scc e.g., G. T. Hermanson 1996, “Antibody Modification and Conjugation, in Bioconjugate Techniques, Academic Press, San Diego) or a carbodiimide conjugation procedure [see e.g., J. March, Advanced Organic Chemistry: Reaction's, Mechanism, and Structure, pp. 349-50 & 372-74 (3d cd.), 1985; B. Neises et al. 1978, Angew Chem., Int. Ed. Engl. 17:522; A. Hassner et al. 1978, Tetrahedron Lett. 4475; E. P. Boden et al. 1986, J. Org. Chem. 50:2394 and L. J. Mathias 1979, Synthesis 561].

A therapeutic moiety can be attached, for example, to the LILRB (e.g. LILRB2, LILRB1) polypeptide or composition, fusion or dimer comprising same of some embodiments of the invention using standard chemical synthesis techniques widely practiced in the art [see e.g., hypertexttransferprotocol://worldwideweb (dot) chemistry (dot) org/portal/Chemistry)], such as using any suitable chemical linkage, direct or indirect, as via a peptide bond (when the functional moiety is a polypeptide), or via covalent bonding to an intervening linker element, such as a linker peptide or other chemical moiety, such as an organic polymer. Chimeric peptides may be linked via bonding at the carboxy (C) or amino (N) termini of the peptides, or via bonding to internal chemical groups such as straight, branched or cyclic side chains, internal carbon or nitrogen atoms, and the like.

According to specific embodiments, the LILRB (e.g. LILRB2, LILRB1) polypeptide or composition, fusion or dimer comprising same comprises a detectable tag. As used herein, in one embodiment the term “detectable tag” refers to any moiety that can be detected by a skilled practitioner using art known techniques. Detectable tags may be peptide sequences. Optionally the detectable tag may be removable by chemical agents or by enzymatic means, such as proteolysis. Detectable tags of some embodiments of the present invention can be used for purification of the polypeptide, the composition, the fusion or the dimer. For example the term “detectable tag” includes chitin binding protein (CBP)-tag, maltose binding protein (MBP)-tag, glutathione-S-transferase (GST)-tag, poly (His)-tag, FLAG tag, Epitope tags, such as, V5-tag, c-myc-tag, and HA-tag, and fluorescence tags such as green fluorescent protein (GFP), red fluorescent protein (RFP), yellow fluorescent protein (YFP), blue fluorescent protein (BFP), and cyan fluorescent protein (CFP); as well as derivatives of these tags, or any tag known in the art. The term “detectable tag” also includes the term “detectable marker”.

According to specific embodiments, the LILRB (e.g. LILRB2, LILRB1) polypeptide or composition, fusion or dimer comprising same comprises a cleavable moiety. Thus, for example, to facilitate recovery, the expressed coding sequence can be engineered to encode the polypeptide of some embodiments of the present invention and fused cleavable moiety. In one embodiment, the polypeptide is designed such that it is readily isolated by affinity chromatography; e.g., by immobilization on a column specific for the cleavable moiety. In one embodiment, a cleavage site is engineered between the polypeptide and the cleavable moiety and the peptide can be released from the chromatographic column by treatment with an appropriate enzyme or agent that specifically cleaves the fusion protein at this site [e.g., see Booth et al., Immunol. Lett. 19:65-70 (1988); and Gardella et al., J. Biol. Chem. 265:15854-15859 (1990)].

According to specific embodiments, each of the moieties in the composition, fusion or dimer disclosed herein may comprise a linker, separating between the moieties, e.g. between the polypeptide (e.g. LILRB, LILRB2, LILRB1, SIRPα) and the dimerizing moiety.

According to other specific embodiments, the composition, fusion or dimer disclosed herein does not comprise a linker between the polypeptide (e.g. LILRB, LILRB2, LILRB1, SIRPα) and the dimerizing moiety.

Any linker known in the art can be used with specific embodiments of the invention.

According to specific embodiments, the linker may be derived from naturally-occurring multi-domain proteins or is an empirical linker as described, for example, in Chichili et al., (2013), Protein Sci. 22 (2): 153-167, Chen et al, (2013), Adv Drug Deliv Rev. 65 (10): 1357-1369, the entire contents of which are hereby incorporated by reference. In some embodiments, the linker may be designed using linker designing databases and computer programs such as those described in Chen et al., (2013), Adv Drug Deliv Rev. 65 (10): 1357-1369 and Crasto et al., (2000), Protein Eng. 13 (5): 309-312, the entire contents of which are hereby incorporated by reference.

According to specific embodiments, the linker is a synthetic linker such as PEG.

According to specific embodiments, the linker may be functional. For example, without limitation, the linker may function to improve the folding and/or stability, improve the expression, improve the pharmacokinetics, and/or improve the bioactivity of the composition. In another example, the linker may function to target the composition to a particular cell type or location.

According to specific embodiments, the linker is a polypeptide.

Non-limiting examples of polypeptide linkers include linkers having the sequence LE, GGGGS (SEQ ID NO: 69), (GGGGS)ₙ (n=1-4) (SEQ ID NO: 70), GGGGSGGGG (SEQ ID NO: 71), (GGGGS)x2 (SEQ ID NO: 33), (GGGGS)x2+GGGG (SEQ ID NO: 72), (GGGGS)x3 (SEQ ID NO: 73), (GGGGS)x4 (SEQ ID NO: 74), (Gly); (SEQ ID NO: 75), (Gly)₆ (SEQ ID NO: 76), (EAAAK)ₙ (n=1-3) (SEQ ID NO: 77)ₙA (EAAAK)ₙA (n=2-5) (SEQ ID NO: 78), AEAAAKEAAAKA (SEQ ID NO: 79), A (EAAAK)+ALEA (EAAAK) 4A (SEQ ID NO: 80), PAPAP (SEQ ID NO: 81), KESGSVSSEQLAQFRSLD (SEQ ID NO: 82), EGKSSGSGSESKST (SEQ ID NO: 83), GSAGSAAGSGEF (SEQ ID NO: 84), and (XP)ₙ, with X designating any amino acid, e.g., Ala, Lys, or Glu.

According to a specific embodiment, the linker is (GGGGS)x2 (SEQ ID NO: 33).

According to specific embodiments, the linker is at a length of one to six amino acids.

According to a specific embodiment, the linker is (GGGGS)x3 (SEQ ID NO: 73).

According to specific embodiments, the linker is at a length of one to six amino acids.

According to specific embodiments, the linker is substantially comprised of glycine and/or serine residues (e.g. about 30%, or about 40%, or about 50%, or about 60%, or about 70%, or about 80%, or about 90%, or about 95%, or about 97% or 100% glycines and serines).

According to specific embodiments, the linker is a single amino acid linker.

In some embodiments of the invention, the one amino acid is glycine.

According to specific embodiments, the composition disclosed herein [e.g. LILRB (e.g. LILRB2, LILRB1) polypeptides, compositions, fusions and dimers comprising same] is soluble (i.e., not immobilized to a synthetic or a naturally occurring surface).

According to specific embodiments, the composition disclosed herein [e.g. LILRB (e.g. LILRB2, LILRB1) polypeptides, compositions, fusions and dimers comprising same] is immobilized to a synthetic or a naturally occurring surface.

As the compositions disclosed herein [e.g. LILRB (e.g. LILRB2, LILRB1) polypeptides, compositions, fusions and dimers comprising same, polynucleotides encoding same or host cells expressing same] comprise a LILRB (e.g. LILRB2, LILRB1) polypeptide, they may be used in methods of activating immune cells, in-vitro, ex-vivo and/or in-vivo.

Thus, according to an aspect of the present invention there is provided a method of activating immune cells, the method comprising in-vitro activating immune cells in the presence of the LILRB (e.g. LILRB2, LILRB1) polypeptide, the composition, fusion polypeptide or dimer comprising same, the polynucleotide encoding same, or the host cell expressing same.

As used herein the term “peripheral mononuclear blood cells (PBMCs)” refers to a blood cell having a single nucleus and includes lymphocytes, monocytes and dendritic cells (DCs).

According to specific embodiments, the PBMCs are selected from the group consisting of dendritic cells (DCs), macrophage, polymorph nuclear cells, T cells, B cells, NK cells and NKT cells.

Methods of obtaining PBMCs are well known in the art, such as drawing whole blood from a subject and collection in a container containing an anti-coagulant (e.g. heparin or citrate); and apheresis. Following, according to specific embodiments, at least one type of PBMCs is purified from the peripheral blood. There are several methods and reagents known to those skilled in the art for purifying PBMCs from whole blood such as leukapheresis, sedimentation, density gradient centrifugation (e.g. ficoll), centrifugal elutriation, fractionation, chemical lysis of e.g. red blood cells (e.g. by ACK), selection of specific cell types using cell surface markers (using e.g. FACS sorter or magnetic cell separation techniques such as are commercially available e.g. from Invitrogen, Stemcell Technologies, Cellpro, Advanced Magnetics, or Miltenyi Biotec.), and depletion of specific cell types by methods such as eradication (e.g. killing) with specific antibodies or by affinity based purification based on negative selection (using e.g. magnetic cell separation techniques, FACS sorter and/or capture ELISA labeling).

Such methods are described for example in THE HANDBOOK OF EXPERIMENTAL IMMUNOLOGY, Volumes 1 to 4, (D. N. Weir, editor) and FLOW CYTOMETRY AND CELL SORTING (A. Radbruch, editor, Springer Verlag, 2000).

According to specific embodiments, the immune cells comprise tumor infiltrating lymphocytes.

As used herein the term “tumor infiltrating lymphocytes (TILs) refers to mononuclear white blood cells that have lest the bloodstream and migrated into a tumor.

According to specific embodiments, the TILs are selected from the group consisting of T cells, B cells, NK cells and monocytes.

Methods of obtaining TILs are well known in the art, such as obtaining tumor samples from a subject by e.g. biopsy or necropsy and preparing a single cell suspension thereof. The single cell suspension can be obtained in any suitable manner, e.g., mechanically (disaggregating the tumor using, e.g., a GentleMACS™ Dissociator, Miltenyi Biotec, Auburn, Calif.) or enzymatically (e.g., collagenase or DNase). Following, the at least one type of TILs can be purified from the cell suspension. There are several methods and reagents known to those skilled in the art for purifying the desired type of TILs, such as selection of specific cell types using cell surface markers (using e.g. FACS sorter or magnetic cell separation techniques such as are commercially available e.g. from Invitrogen, Stemcell Technologies, Cellpro, Advanced Magnetics, or Miltenyi Biotec.), and depletion of specific cell types by methods such as eradication (e.g. killing) with specific antibodies or by affinity based purification based on negative selection (using e.g. magnetic cell separation techniques, FACS sorter and/or capture ELISA labeling). Such methods are described for example in THE HANDBOOK OF EXPERIMENTAL IMMUNOLOGY, Volumes 1 to 4, (D. N. Weir, editor) and FLOW CYTOMETRY AND CELL SORTING (A. Radbruch, editor, Springer Verlag, 2000).

According to specific embodiments, the immune cells comprise phagocytic cells.

As used herein, the term “phagocytic cells” refer to a cell that is capable of phagocytosis and include both professional and non-professional phagocytic cells. Methods of analyzing phagocytosis are well known in the art and include for examples killing assays, flow cytometry and/or microscopic evaluation (live cell imaging, fluorescence microscopy, confocal microscopy, electron microscopy). According to specific embodiments, the phagocytic cells are selected from the group consisting of monocytes, dendritic cells (DCs) and granulocytes.

According to specific embodiments, the phagocytes comprise granulocytes.

According to specific embodiments, the phagocytes comprise monocytes.

According to specific embodiments, the immune cells comprise monocytes.

According to specific embodiments, the term “monocytes” refers to both circulating monocytes and to macrophages (also referred to as mononuclear phagocytes) present in a tissue.

According to specific embodiments, the monocytes comprise macrophages. Typically, cell surface phenotype of macrophages include CD14, CD40, CD11b, CD64, F4/80 (mice)/EMR1 (human), lysozyme M, MAC-1/MAC-3 and CD68.

According to specific embodiments, the monocytes comprise circulating monocytes. Typically, cell surface phenotypes of circulating monocytes include CD14 and CD16 (e.g. CD14++CD16−, CD14+CD16++, CD14++CD16+).

According to specific embodiments, the immune cells comprise DCs

As used herein the term “dendritic cells (DCs)” refers to any member of a diverse population of morphologically similar cell types found in lymphoid or non-lymphoid tissues. DCs are a class of professional antigen presenting cells, and have a high capacity for sensitizing HLA-restricted T cells. DCs include, for example, plasmacytoid dendritic cells, myeloid dendritic cells (including immature and mature dendritic cells), Langerhans cells, interdigitating cells, follicular dendritic cells. Dendritic cells may be recognized by function, or by phenotype, particularly by cell surface phenotype. These cells are characterized by their distinctive morphology having veil-like projections on the cell surface, intermediate to high levels of surface HLA-class II expression and ability to present antigen to T cells, particularly to naive T cells (See Steinman R, et al., Ann. Rev. Immunol. 1991; 9:271-196.). Typically, cell surface phenotype of DCs include CD1a+, CD4+, CD86+, or HLA-DR. The term DCs encompasses both immature and mature DCs.

According to specific embodiments, the immune cells comprise granulocytes.

As used herein, the term “granulocytes” refer to polymorphonuclear leukocytes characterized by the presence of granules in their cytoplasm.

According to specific embodiments, the granulocytes comprise neutrophils.

According to specific embodiments, the granulocytes comprise mast-cells.

According to specific embodiments the immune cells comprise T cells.

As used herein, the term “T cells” refers to a differentiated lymphocyte with a CD3+, T cell receptor (TCR)+ having either CD4+ or CD8+ phenotype. The T cell may be either an effector or a regulatory T cell.

As used herein, the term “effector T cells” refers to a T cell that activates or directs other immune cells e.g. by producing cytokines or has a cytotoxic activity e.g., CD4+, Th1/Th2, CD8+ cytotoxic T lymphocyte.

As used herein, the term “regulatory T cell” or “Treg” refers to a T cell that negatively regulates the activation of other T cells, including effector T cells, as well as innate immune system cells. Treg cells are characterized by sustained suppression of effector T cell responses. According to a specific embodiment, the Treg is a CD4+CD25+ Foxp3+ T cell.

According to specific embodiments, the T cells are CD4+ T cells.

According to other specific embodiments, the T cells are CD8+ T cells.

According to specific embodiments, the T cells are memory T cells. Non-limiting examples of memory T cells include effector memory CD4+ T cells with a CD3+/CD4+/CD45RA-/CCR7-phenotype, central memory CD4+ T cells with a CD3+/CD4+/CD45RA-/CCR7+ phenotype, effector memory CD8+ T cells with a CD3+/CD8+CD45RA-/CCR7-phenotype and central memory CD8+ T cells with a CD3+/CD8+CD45RA-/CCR7+ phenotype.

According to specific embodiments, the T cells comprise engineered T cells transduced with a nucleic acid sequence encoding an expression product of interest.

According to specific embodiments, the expression product of interest is a T cell receptor (TCR) or a chimeric antigen receptor (CAR).

As used herein the phrase “transduced with a nucleic acid sequence encoding a TCR” or “transducing with a nucleic acid sequence encoding a TCR” refers to cloning of variable α- and β-chains from T cells with specificity against a desired antigen presented in the context of MHC. Methods of transducing with a TCR are known in the art and are disclosed e.g. in Nicholson et al. Adv Hematol. 2012; 2012:404081; Wang and Rivière Cancer Gene Ther. 2015 Mar.; 22 (2): 85-94); and Lamers et al, Cancer Gene Therapy (2002) 9, 613-623.

As used herein, the phrase “transduced with a nucleic acid sequence encoding a CAR” or “transducing with a nucleic acid sequence encoding a CAR” refers to cloning of a nucleic acid sequence encoding a chimeric antigen receptor (CAR), wherein the CAR comprises an antigen recognition moiety and a T-cell activation moiety. A chimeric antigen receptor (CAR) is an artificially constructed hybrid protein or polypeptide containing an antigen binding domain of an antibody (e.g., a single chain variable fragment (scFv)) linked to T-cell signaling or T-cell activation domains. Method of transducing with a CAR are known in the art and are disclosed e.g. in Davila et al. Oncoimmunology. 2012 Dec. 1; 1 (9): 1577-1583; Wang and Rivière Cancer Gene Ther. 2015 Mar.; 22 (2): 85-94); Maus et al. Blood. 2014 Apr. 24; 123 (17): 2625-35; Porter D L The New England journal of medicine. 2011, 365 (8): 725-733; Jackson H J, Nat Rev Clin Oncol. 2016; 13 (6): 370-383; and Globerson-Levin et al. Mol Ther. 2014; 22 (5): 1029-1038.

According to specific embodiments, the immune cells comprise B cells.

As used herein the term “B cells” refers to a lymphocyte with a B cell receptor (BCR)+, CD19+ and or B220+ phenotype. B cells are characterized by their ability to bind a specific antigen and elicit a humoral response.

According to specific embodiments, the immune cells comprise NK cells.

As used herein the term “NK cells” refers to differentiated lymphocytes with a CD16+CD56+ and/or CD57+ TCR-phenotype. NK are characterized by their ability to bind to and kill cells that fail to express “self” MHC/HLA antigens by the activation of specific cytolytic enzymes, the ability to kill tumor cells or other diseased cells that express a ligand for NK activating receptors, and the ability to release protein molecules called cytokines that stimulate or inhibit the immune response.

According to specific embodiments, the immune cells comprise NKT cells.

As used herein the term “NKT cells” refers to a specialized population of T cells that express a semi-invariant αβ T-cell receptor, but also express a variety of molecular markers that are typically associated with NK cells, such as NK1.1. NKT cells include NK1.1+ and NK1.1−, as well as CD4+, CD4−, CD8+ and CD8− cells. The TCR on NKT cells is unique in that it recognizes glycolipid antigens presented by the MHC I-like molecule CD1d. NKT cells can have either protective or deleterious effects due to their abilities to produce cytokines that promote either inflammation or immune tolerance.

According to specific embodiments, the immune cells are obtained from a healthy subject.

According to specific embodiments, the immune cells are obtained from a subject suffering from a pathology (e.g. cancer).

According to specific embodiments, activating is in the presence of cells expressing HLA-G.

According to specific embodiments, the cells expressing HLA-G comprise pathologic (diseased) cells, e.g. cancer cells.

According to specific embodiments, the activating is in the presence of a stimulatory agent capable of at least transmitting a primary activating signal [e.g. ligation of the T-Cell Receptor (TCR) with the Major Histocompatibility Complex (MHC)/peptide complex on the Antigen Presenting Cell (APC)] resulting in cellular proliferation, maturation, cytokine production, phagocytosis and/or induction of regulatory or effector functions of the immune cell. According to specific embodiments, the stimulator agent can also transmit a secondary co-stimulatory signal.

The stimulatory agent can activate the immune cells in an antigen-dependent or -independent (i.e. polyclonal) manner.

Methods of determining the amount of the stimulatory agent and the ratio between the stimulatory agent and the immune cells are well within the capabilities of the skilled in the art and thus are not specified herein.

According to specific embodiments, the immune cells are purified following the activation.

Thus, the present invention also contemplates isolated immune cells obtainable according to the methods of the present invention.

According to specific embodiments, the immune cells used and/or obtained according to the present invention can be freshly isolated, stored e.g., cryopreserved (i.e. frozen) at e.g. liquid nitrogen temperature at any stage for long periods of time (e.g., months, years) for future use; and cell lines.

Methods of cryopreservation are commonly known by one of ordinary skill in the art and are disclosed e.g. in International Patent Application Publication Nos. WO2007054160 and WO 2001039594 and US Patent Application Publication No. US20120149108.

According to specific embodiments, the cells obtained according to the present invention can be stored in a cell bank or a depository or storage facility.

Consequently, the present teachings further suggest the use of the isolated immune cells and the methods of the present invention as, but not limited to, a source for adoptive immune cells therapies.

Thus, according to specific embodiments, a method of the present invention comprises adoptively transferring the immune cells following said activating to a subject in need thereof.

According to specific embodiments, there is provided the immune cells obtainable according to the methods of the present invention for use in adoptive cell therapy.

The cells used according to specific embodiments of the present invention may be autologous or non-autologous; they can be syngeneic or non-syngeneic: allogeneic or xenogeneic to the subject; each possibility represents a separate embodiment of the present invention.

The present teachings also contemplate the use of the compositions of the present invention [e.g. the LILRB (e.g. LILRB2, LILRB1) polypeptide, a composition, fusion or dimer comprising same, a polynucleotide encoding same or a host cell expressing same] in methods of treating a disease associated with pathologic cells expressing HLA-G.

Thus, according to an aspect of the present invention, there is provided a method of treating a disease associated with pathologic cells expressing HLA-G in a subject in need thereof, the method comprising administering to the subject a therapeutically effective amount of the LILRB (e.g. LILRB2, LILRB1) polypeptide, the composition, the fusion polypeptide or the dimer, the polynucleotide encoding same, or the host cell expressing same disclosed herein, thereby treating the disease in the subject.

According to an additional or an alternative aspect of the present invention, there is provided the LILRB (e.g. LILRB2, LILRB1) polypeptide, the composition, the fusion polypeptide or the dimer, the polynucleotide encoding same, or the host cell expressing same disclosed herein, for use in treating a disease associated with pathologic cells expressing HLA-G in a subject in need thereof.

As used herein, the term “subject” refers to a human or non-human individual having an MHC system, such as the HLA system in humans. The subject may be of any gender and of any age.

According to specific embodiments, the subject is a human subject.

According to specific embodiments, the subject is diagnosed with a disease (e.g., cancer) or is at risk of developing a disease (e.g., cancer).

According to specific embodiments, pathologic cells of the subject present HLA-G at a level above a predetermined threshold, as further described hereinbelow.

Thus, according to specific embodiments, the methods disclosed herein further comprise determining a level of HLA-G in a biological sample of the subject e.g. prior to administering of the LILRB (e.g. LILRB2, LILRB1) polypeptide, the composition, fusion or dimer comprising same, the polynucleotide encoding same or host cell expressing same and treating the subject accordingly.

As used herein the term “treating” refers to inhibiting, preventing or arresting the development of a pathology (disease, disorder, or condition e.g., cancer) and/or causing the reduction, remission, or regression of a pathology. Those of skill in the art will understand that various methodologies and assays can be used to assess the development of a pathology, and similarly, various methodologies and assays may be used to assess the reduction, remission or regression of a pathology.

According to specific embodiments, treatment may be evaluated by a decrease in tumor volume, a decrease in the number of tumor cells, a decrease in the number of metastases, an increase in life expectancy, or amelioration of various physiological symptoms associated with the cancerous condition.

As used herein the phrase “a disease associated with pathologic cells expressing HLA-G” refers to a disease in which pathologic cells presenting HLA-G drive onset and/or progression of the disease.

According to specific embodiments, pathologic cells present HLA-G at a level above a predetermined threshold.

Such a predetermined threshold can be experimentally determined by comparing presentation levels in a biological sample derived from subjects diagnosed with the disease (e.g. caner) to a biological sample obtained from healthy subjects (e.g., not having the disease e.g. cancer). Alternatively or additionally, such a predetermined threshold can be experimentally determined by comparing presentation levels in pathologic cells (e.g. cancer cells) to presentation levels in healthy cells obtained from the same subject. Alternatively, such a level can be obtained from the scientific literature and from databases.

According to specific embodiments, the level above a predetermined threshold is statistically significant.

According to specific embodiments the increase from a predetermined threshold is at least 5%, at least 10%, at least 20%, at least 30%, at least 40%, at least 50%, at least 60%, at least 70%, at least 80%, at least 90%, at least 95%, at least 100% or more, higher than about 2 times, higher than about three times, higher than about four time, higher than about five times, higher than about six times, higher than about seven times, higher than about eight times, higher than about nine times, higher than about 20 times, higher than about 50 times, higher than about 100 times, higher than about 200 times, higher than about 350, higher than about 500 times, higher than about 1000 times, or more as compared to the control sample as measured using the same assay.

Methods of determining HLA-G expression are known in the art, and include e.g. flow cytometry, immunohistochemistry, ELISA and the like.

Alternatively or additionally, such a predetermined threshold is a detectable level of HLA-G as determined by ELISA, immunohistochemistry or flow cytometry.

Non-limiting Examples of diseases that can be treated with specific embodiments of the invention include cancer, viral infections (e.g. HCMV, HSV-1, RABV, HCV, IAV and HIV-1), auto-immune and inflammatory diseases, following allo-transplantation, GVHD.

According to specific embodiments, the disease is cancer.

Cancers which may be treated by some embodiments of the invention can be any solid or non-solid tumor, cancer metastasis and/or a pre-cancer.

According to specific embodiments, the cancer is a malignant cancer.

Examples of cancer include but are not limited to, carcinoma, blastoma, sarcoma and lymphoma. More particular examples of such cancers include, but are not limited to, tumors of the gastrointestinal tract (colon carcinoma, rectal carcinoma, colorectal carcinoma, colorectal cancer, colorectal adenoma, hereditary nonpolyposis type 1, hereditary nonpolyposis type 2, hereditary nonpolyposis type 3, hereditary nonpolyposis type 6; colorectal cancer, hereditary nonpolyposis type 7, small and/or large bowel carcinoma, esophageal carcinoma, tylosis with esophageal cancer, stomach carcinoma, pancreatic carcinoma, pancreatic endocrine tumors), endometrial carcinoma, dermatofibrosarcoma protuberans, gallbladder carcinoma, Biliary tract tumors, prostate cancer, prostate adenocarcinoma, renal cancer (e.g., Wilms' tumor type 2 or type 1), liver cancer (e.g., hepatoblastoma, hepatocellular carcinoma, hepatocellular cancer), bladder cancer, embryonal rhabdomyosarcoma, germ cell tumor, trophoblastic tumor, testicular germ cells tumor, immature teratoma of ovary, uterine, epithelial ovarian, sacrococcygeal tumor, choriocarcinoma, placental site trophoblastic tumor, epithelial adult tumor, ovarian carcinoma, serous ovarian cancer, ovarian sex cord tumors, cervical carcinoma, uterine cervix carcinoma, small-cell and non-small cell lung carcinoma, nasopharyngeal, breast carcinoma (e.g., ductal breast cancer, invasive intraductal breast cancer, sporadic; breast cancer, susceptibility to breast cancer, type 4 breast cancer, breast cancer-1, breast cancer-3; breast-ovarian cancer), squamous cell carcinoma (e.g., in head and neck), neurogenic tumor, astrocytoma, ganglioblastoma, neuroblastoma, lymphomas (e.g., Hodgkin's disease, non-Hodgkin's lymphoma, B cell, Burkitt, cutaneous T cell, histiocytic, lymphoblastic, T cell, thymic), gliomas, adenocarcinoma, adrenal tumor, hereditary adrenocortical carcinoma, brain malignancy (tumor), various other carcinomas (e.g., bronchogenic large cell, ductal, Ehrlich-Lettre ascites, epidermoid, large cell, Lewis lung, medullary, mucoepidermoid, oat cell, small cell, spindle cell, spinocellular, transitional cell, undifferentiated, carcinosarcoma, choriocarcinoma, cystadenocarcinoma), ependimoblastoma, epithelioma, erythroleukemia (e.g., Friend, lymphoblast), fibrosarcoma, giant cell tumor, glial tumor, glioblastoma (e.g., multiforme, astrocytoma), glioma hepatoma, heterohybridoma, heteromyeloma, histiocytoma, hybridoma (e.g., B cell), hypernephroma, insulinoma, islet tumor, keratoma, leiomyoblastoma, leiomyosarcoma, leukemia (e.g., acute lymphatic, acute lymphoblastic, acute lymphoblastic pre-B cell, acute lymphoblastic T cell leukemia, acute-megakaryoblastic, monocytic, acute myelogenous, acute myeloid, acute myeloid with eosinophilia, B cell, basophilic, chronic myeloid, chronic, B cell, eosinophilic, Friend, granulocytic or myelocytic, hairy cell, lymphocytic, megakaryoblastic, monocytic, monocytic-macrophage, myeloblastic, myeloid, myelomonocytic, plasma cell, pre-B cell, promyelocytic, subacute, T cell, lymphoid neoplasm, predisposition to myeloid malignancy, acute nonlymphocytic leukemia), lymphosarcoma, melanoma, mammary tumor, mastocytoma, medulloblastoma, mesothelioma, metastatic tumor, monocyte tumor, multiple myeloma, myelodysplastic syndrome, myeloma, nephroblastoma, nervous tissue glial tumor, nervous tissue neuronal tumor, neurinoma, neuroblastoma, oligodendroglioma, osteochondroma, osteomyeloma, osteosarcoma (e.g., Ewing's), papilloma, transitional cell, pheochromocytoma, pituitary tumor (invasive), plasmacytoma, retinoblastoma, rhabdomyosarcoma, sarcoma (e.g., Ewing's, histiocytic cell, Jensen, osteogenic, reticulum cell), schwannoma, subcutaneous tumor, teratocarcinoma (e.g., pluripotent), teratoma, testicular tumor, thymoma and trichoepithelioma, gastric cancer, fibrosarcoma, glioblastoma multiforme; multiple glomus tumors, Li-Fraumeni syndrome, liposarcoma, lynch cancer family syndrome II, male germ cell tumor, mast cell leukemia, medullary thyroid, multiple meningioma, endocrine neoplasia myxosarcoma, paraganglioma, familial nonchromaffin, pilomatricoma, papillary, familial and sporadic, rhabdoid predisposition syndrome, familial, rhabdoid tumors, soft tissue sarcoma, and Turcot syndrome with glioblastoma.

According to specific embodiments, the cancer is a pre-malignant cancer.

Pre-cancers are well characterized and known in the art (refer, for example, to Berman J J. and Henson D E., 2003. Classifying the pre-cancers: a metadata approach. BMC Med Inform Decis Mak. 3:8). Examples of pre-cancers include, but are not limited to, acquired small pre-cancers, acquired large lesions with nuclear atypia, precursor lesions occurring with inherited hyperplastic syndromes that progress to cancer, and acquired diffuse hyperplasias and diffuse metaplasias. Non-limiting examples of small pre-cancers include HGSIL (High grade squamous intraepithelial lesion of uterine cervix), AIN (anal intraepithelial neoplasia), dysplasia of vocal cord, aberrant crypts (of colon), PIN (prostatic intraepithelial neoplasia).

Non-limiting examples of acquired large lesions with nuclear atypia include tubular adenoma, AILD (angioimmunoblastic lymphadenopathy with dysproteinemia), atypical meningioma, gastric polyp, large plaque parapsoriasis, myelodysplasia, papillary transitional cell carcinoma in-situ, refractory anemia with excess blasts, and Schneiderian papilloma. Non-limiting examples of precursor lesions occurring with inherited hyperplastic syndromes that progress to cancer include atypical mole syndrome, C cell adenomatosis and MEA. Non-limiting examples of acquired diffuse hyperplasias and diffuse metaplasias include Paget's disease of bone and ulcerative colitis.

According to specific embodiments, the cancer is selected from the group consisting of pancreatic, breast, skin, colorectal, gastric and ovarian cancer.

According to specific embodiments, the compositions disclosed herein [e.g. LILRB (e.g. LILRB2, LILRB2) polypeptide, composition, fusion or dimer comprising same, polynuelcodie encoding same and/or host-cell expressing same] can be administered to a subject in combination with other established or experimental therapeutic regimen to treat the disease including, but not limited to analgesics, chemotherapeutic agents, radiotherapeutic agents, cytotoxic therapies (conditioning), hormonal therapy, antibodies and other treatment regimens (e.g., surgery) which are well known in the art.

According to specific embodiments, the therapeutic agent administered in combination with the composition of some embodiments of the invention comprises an antibody.

According to specific embodiments, the compositions disclosed herein [e.g. LILRB (e.g. LILRB2, LILRB1) polypeptide, composition, fusion or dimer comprising same, polynucleotide encoding same and/or host-cell expressing same] can be administered to a subject in combination with adoptive cell transplantation such as, but not limited to transplantation of bone marrow cells, hematopoietic stem cells, PBMCs, cord blood stem cells and/or induced pluripotent stem cells.

According to specific embodiments, the therapeutic agent administered in combination with the composition of some embodiments of the invention comprises an anti-cancer agent.

According to specific embodiments, the therapeutic agent administered in combination with the composition of some embodiments of the invention comprises an anti-infection agent (e.g. antibiotics and anti-viral agents).

According to specific embodiments the combination therapy has an additive effect.

According to specific embodiments, the combination therapy has a synergistic effect.

According to another aspect of the present invention there is provided an article of manufacture comprising a packaging material packaging a therapeutic agent for treating a disease; and the LILRB (e.g. LILRB2, LILRB1) polypeptide, composition, fusion or dimer comprising same, polynucleotide encoding same and/or host-cell expressing same.

According to specific embodiments, the article of manufacture is identified for the treatment of a disease associated with pathologic cells expressing HLA-G e.g. cancer.

According to specific embodiments, the therapeutic agent for treating said disease; and the LILRB (e.g. LILRB2, LILRB1) polypeptide, composition, fusion or dimer comprising same, polynucleotide encoding same and/or host-cell expressing same are packaged in separate containers.

According to specific embodiments, the therapeutic agent for treating said disease; and the LILRB (e.g. LILRB2, LILRB1) polypeptide, composition, fusion or dimer comprising same, polynucleotide encoding same and/or host-cell expressing same are packaged in a co-formulation.

As used herein, the terms “amino acid sequence”, “protein”, “peptide”, “polypeptide” and “proteinaceous moiety”, which are interchangeably used herein, encompass native peptides (either degradation products, synthetically synthesized peptides or recombinant peptides) and peptidomimetics (typically, synthetically synthesized peptides), as well as peptoids and semipeptoids which are peptide analogs, which may have, for example, modifications rendering the peptides more stable while in a body or more capable of penetrating into cells. Such modifications include, but are not limited to N terminus modification, C terminus modification, peptide bond modification, backbone modifications, and residue modification. Methods for preparing peptidomimetic compounds are well known in the art and are specified, for example, in Quantitative Drug Design, C. A. Ramsden Gd., Chapter 17.2, F. Choplin Pergamon Press (1992), which is incorporated by reference as if fully set forth herein. Further details in this respect are provided hereinunder.

Peptide bonds (—CO—NH—) within the peptide may be substituted, for example, by N-methylated amide bonds (—N(CH3)-CO—), ester bonds (—C(═O)—O—), ketomethylene bonds (—CO—CH2-), sulfinylmethylene bonds (—S(═O)—CH2-), α-aza bonds (—NH—N(R)—CO—), wherein R is any alkyl (e.g., methyl), amine bonds (—CH2-NH—), sulfide bonds (—CH2-S—), ethylene bonds (—CH2-CH2-), hydroxyethylene bonds (—CH(OH)—CH2-), thioamide bonds (—CS—NH—), olefinic double bonds (—CH—CH—), fluorinated olefinic double bonds (—CF═CH—), retro amide bonds (—NH—CO—), peptide derivatives (—N(R)—CH2-CO—), wherein R is the “normal” side chain, naturally present on the carbon atom.

These modifications can occur at any of the bonds along the peptide chain and even at several (2-3) bonds at the same time.

Natural aromatic amino acids, Trp, Tyr and Phe, may be substituted by non-natural aromatic amino acids such as 1,2,3,4-tetrahydroisoquinoline-3-carboxylic acid (Tic), naphthylalanine, ring-methylated derivatives of Phe, halogenated derivatives of Phe or O-methyl-Tyr.

The peptides of some embodiments of the invention may also include one or more modified amino acids or one or more non-amino acid monomers (e.g. fatty acids, complex carbohydrates etc.).

The term “amino acid” or “amino acids” is understood to include the 20 naturally occurring amino acids; those amino acids often modified post-translationally in vivo, including, for example, hydroxyproline, phosphoserine and phosphothreonine; and other unusual amino acids including, but not limited to, 2-aminoadipic acid, hydroxylysine, isodesmosine, nor-valine, nor-leucine and ornithine. Furthermore, the term “amino acid” includes both D- and L-amino acids.

Tables 4 and 5 below list naturally occurring amino acids (Table 4), and non-conventional or modified amino acids (e.g., synthetic, Table 5) which can be used with some embodiments of the invention.

TABLE 4

| | Three-Letter | One-letter |
|---|---|---|
| Amino Acid | Abbreviation | Symbol |
| Alanine | Ala | A |
| Arginine | Arg | R |
| Asparagine | Asn | N |
| Aspartic acid | Asp | D |
| Cysteine | Cys | C |
| Glutamine | Gln | Q |
| Glutamic Acid | Glu | E |
| Glycine | Gly | G |
| Histidine | His | H |
| Isoleucine | Ile | I |
| Leucine | Leu | L |
| Lysine | Lys | K |
| Methionine | Met | M |
| Phenylalanine | Phe | F |
| Proline | Pro | P |
| Serine | Ser | S |
| Threonine | Thr | T |
| Tryptophan | Trp | W |
| Tyrosine | Tyr | Y |
| Valine | Val | V |
| Any amino acid as above | Xaa | X |

TABLE 5

| Non-conventional | | Non-conventional | |
|---|---|---|---|
| amino acid | Code | amino acid | Code |
| ornithine | Orn | hydroxyproline | Hyp |
| α-aminobutyric acid | Abu | aminonorbornyl- | Norb |
| | | carboxylate | |
| D-alanine | Dala | aminocyclopropane- | Cpro |
| | | carboxylate | |
| D-arginine | Darg | N-(3-guanidinopropyl)glycine | Narg |
| D-asparagine | Dasn | N-(carbamylmethyl)glycine | Nasn |
| D-aspartic acid | Dasp | N-(carboxymethyl)glycine | Nasp |
| D-cysteine | Dcys | N-(thiomethyl)glycine | Ncys |
| D-glutamine | Dgln | N-(2-carbamylethyl)glycine | Ngln |
| D-glutamic acid | Dglu | N-(2-carboxyethyl)glycine | Nglu |
| D-histidine | Dhis | N-(imidazolylethyl)glycine | Nhis |
| D-isoleucine | Dile | N-(1-methylpropyl)glycine | Nile |
| D-leucine | Dleu | N-(2-methylpropyl)glycine | Nleu |
| D-lysine | Dlys | N-(4-aminobutyl)glycine | Nlys |
| D-methionine | Dmet | N-(2-methylthioethyl)glycine | Nmet |
| D-ornithine | Dorn | N-(3-aminopropyl)glycine | Norn |
| D-phenylalanine | Dphe | N-benzylglycine | Nphe |
| D-proline | Dpro | N-(hydroxymethyl)glycine | Nser |
| D-serine | Dser | N-(1-hydroxyethyl)glycine | Nthr |
| D-threonine | Dthr | N-(3-indolylethyl) glycine | Nhtrp |
| D-tryptophan | Dtrp | N-(p-hydroxyphenyl)glycine | Ntyr |
| D-tyrosine | Dtyr | N-(1-methylethyl)glycine | Nval |
| D-valine | Dval | N-methylglycine | Nmgly |
| D-N-methylalanine | Dnmala | L-N-methylalanine | Nmala |
| D-N-methylarginine | Dnmarg | L-N-methylarginine | Nmarg |
| D-N-methylasparagine | Dnmasn | L-N-methylasparagine | Nmasn |
| D-N-methylasparatate | Dnmasp | L-N-methylaspartic acid | Nmasp |
| D-N-methylcysteine | Dnmcys | L-N-methylcysteine | Nmcys |
| D-N-methylglutamine | Dnmgln | L-N-methylglutamine | Nmgln |
| D-N-methylglutamate | Dnmglu | L-N-methylglutamic acid | Nmglu |
| D-N-methylhistidine | Dnmhis | L-N-methylhistidine | Nmhis |
| D-N-methylisoleucine | Dnmile | L-N-methylisolleucine | Nmile |
| D-N-methylleucine | Dnmleu | L-N-methylleucine | Nmleu |
| D-N-methyllysine | Dnmlys | L-N-methyllysine | Nmlys |
| D-N-methylmethionine | Dnmmet | L-N-methylmethionine | Nmmet |
| D-N-methylornithine | Dnmorn | L-N-methylornithine | Nmorn |
| D-N-methylphenylalanine | Dnmphe | L-N-methylphenylalanine | Nmphe |
| D-N-methylproline | Dnmpro | L-N-methylproline | Nmpro |
| D-N-methylserine | Dnmser | L-N-methylserine | Nmser |
| D-N-methylthreonine | Dnmthr | L-N-methylthreonine | Nmthr |
| D-N-methyltryptophan | Dnmtrp | L-N-methyltryptophan | Nmtrp |
| D-N-methyltyrosine | Dnmtyr | L-N-methyltyrosine | Nmtyr |
| D-N-methylvaline | Dnmval | L-N-methylvaline | Nmval |
| L-norleucine | Nle | L-N-methylnorleucine | Nmnle |
| L-norvaline | Nva | L-N-methylnorvaline | Nmnva |
| L-ethylglycine | Etg | L-N-methyl-ethylglycine | Nmetg |
| L-t-butylglycine | Tbug | L-N-methyl-t-butylglycine | Nmtbug |
| L-homophenylalanine | Hphe | L-N-methyl-homophenylalanine | Nmhphe |
| α-naphthylalanine | Anap | N-methyl-α-naphthylalanine | Nmanap |
| penicillamine | Pen | N-methylpenicillamine | Nmpen |
| γ-aminobutyric acid | Gabu | N-methyl-γ-aminobutyrate | Nmgabu |
| cyclohexylalanine | Chexa | N-methyl-cyclohexylalanine | Nmchexa |
| cyclopentylalanine | Cpen | N-methyl-cyclopentylalanine | Nmcpen |
| α-amino-α-methylbutyrate | Aabu | N-methyl-α-amino-α- | Nmaabu |
| | | methylbutyrate | |
| α-aminoisobutyric acid | Aib | N-methyl-α-aminoisobutyrate | Nmaib |
| D-α-methylarginine | Dmarg | L-α-methylarginine | Marg |
| D-α-methylasparagine | Dmasn | L-α-methylasparagine | Masn |
| D-α-methylaspartate | Dmasp | L-α-methylaspartate | Masp |
| D-α-methylcysteine | Dmcys | L-α-methylcysteine | Mcys |
| D-α-methylglutamine | Dmgln | L-α-methylglutamine | Mgln |
| D-α-methyl glutamic acid | Dmglu | L-α-methylglutamate | Mglu |
| D-α-methylhistidine | Dmhis | L-α-methylhistidine | Mhis |
| D-α-methylisoleucine | Dmile | L-α-methylisoleucine | Mile |
| D-α-methylleucine | Dmleu | L-α-methylleucine | Mleu |
| D-α-methyllysine | Dmlys | L-α-methyllysine | Mlys |
| D-α-methylmethionine | Dmmet | L-α-methylmethionine | Mmet |
| D-α-methylornithine | Dmorn | L-α-methylornithine | Morn |
| D-α-methylphenylalanine | Dmphe | L-α-methylphenylalanine | Mphe |
| D-α-methylproline | Dmpro | L-α-methylproline | Mpro |
| D-α-methylserine | Dmser | L-α-methylserine | Mser |
| D-α-methylthreonine | Dmthr | L-α-methylthreonine | Mthr |
| D-α-methyltryptophan | Dmtrp | L-α-methyltryptophan | Mtrp |
| D-α-methyltyrosine | Dmtyr | L-α-methyltyrosine | Mtyr |
| D-α-methylvaline | Dmval | L-α-methylvaline | Mval |
| N-cyclobutylglycine | Ncbut | L-α-methylnorvaline | Mnva |
| N-cycloheptylglycine | Nchep | L-α-methylethylglycine | Metg |
| N-cyclohexylglycine | Nchex | L-α-methyl-t-butylglycine | Mtbug |
| N-cyclodecylglycine | Ncdec | L-α-methyl-homophenylalanine | Mhphe |
| N-cyclododecylglycine | Ncdod | α-methyl-α-naphthylalanine | Manap |
| N-cyclooctylglycine | Ncoct | α-methylpenicillamine | Mpen |
| N-cyclopropylglycine | Ncpro | α-methyl-γ-aminobutyrate | Mgabu |
| N-cycloundecylglycine | Ncund | α-methyl-cyclohexylalanine | Mchexa |
| N-(2-aminoethyl)glycine | Naeg | α-methyl-cyclopentylalanine | Mcpen |
| N-(2,2-diphenylethyl)glycine | Nbhm | N-(N-(2,2-diphenylethyl) | Nnbhm |
| | | carbamylmethyl-glycine | |
| N-(3,3- | Nbhe | N-(N-(3,3-diphenylpropyl) | Nnbhe |
| diphenylpropyl)glycine | | carbamylmethyl-glycine | |
| 1-carboxy-1-(2,2-diphenyl | Nmbc | 1,2,3,4-tetrahydroisoquinoline- | Tic |
| ethylamino)cyclopropane | | 3-carboxylic acid | |
| phosphoserine | pSer | phosphothreonine | pThr |
| phosphotyrosine | pTyr | O-methyl-tyrosine | |
| 2-aminoadipic acid | | hydroxylysine | |

The polypeptides of some embodiments of the invention are preferably utilized in a linear form, although it will be appreciated that in cases where cyclicization does not severely interfere with peptide characteristics, cyclic forms of the peptide can also be utilized.

Since the present polypeptides are preferably utilized in therapeutics which require the polypeptide to be in soluble form, the polypeptides of some embodiments of the invention include one or more non-natural or natural polar amino acids, including but not limited to serine and threonine which are capable of increasing peptide solubility due to their hydroxyl-containing side chain.

The amino acids of the polypeptides of the present invention may be substituted either conservatively or non-conservatively.

The term “conservative substitution” as used herein, refers to the replacement of an amino acid present in the native sequence in the peptide with a naturally or non-naturally occurring amino or a peptidomimetics having similar steric properties. Where the side-chain of the native amino acid to be replaced is either polar or hydrophobic, the conservative substitution should be with a naturally occurring amino acid, a non-naturally occurring amino acid or with a peptidomimetic moiety which is also polar or hydrophobic (in addition to having the same steric properties as the side-chain of the replaced amino acid).

As naturally occurring amino acids are typically grouped according to their properties, conservative substitutions by naturally occurring amino acids can be easily determined bearing in mind the fact that in accordance with the invention replacement of charged amino acids by sterically similar non-charged amino acids are considered as conservative substitutions.

For producing conservative substitutions by non-naturally occurring amino acids it is also possible to use amino acid analogs (synthetic amino acids) well known in the art. A peptidomimetic of the naturally occurring amino acid is well documented in the literature known to the skilled practitioner.

When affecting conservative substitutions, the substituting amino acid should have the same or a similar functional group in the side chain as the original amino acid.

Conservative substitution tables providing functionally similar amino acids are well known in the art. Guidance concerning which amino acid changes are likely to be phenotypically silent can also be found in Bowie et al., 1990, Science 247:1306 1310. Such conservatively modified variants are in addition to and do not exclude polymorphic variants, interspecies homologs, and alleles. Typical conservative substitutions include but are not limited to: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine(S), Threonine (T); and 8) Cysteine (C), Methionine (M) (see, e.g., Creighton, Proteins (1984)). Amino acids can be substituted based upon properties associated with side chains, for example, amino acids with polar side chains may be substituted, for example, Serine(S) and Threonine (T); amino acids based on the electrical charge of a side chains, for example, Arginine (R) and Histidine (H); and amino acids that have hydrophobic side chains, for example, Valine (V) and Leucine (L). As indicated, changes are typically of a minor nature, such as conservative amino acid substitutions that do not significantly affect the folding or activity of the protein.

The phrase “non-conservative substitutions” as used herein refers to replacement of the amino acid as present in the parent sequence by another naturally or non-naturally occurring amino acid, having different electrochemical and/or steric properties. Thus, the side chain of the substituting amino acid can be significantly larger (or smaller) than the side chain of the native amino acid being substituted and/or can have functional groups with significantly different electronic properties than the amino acid being substituted. Examples of non-conservative substitutions of this type include the substitution of phenylalanine or cycohexylmethyl glycine for alanine, isoleucine for glycine, or —NH—CH [(—CH2)₅—COOH]—CO— for aspartic acid. Those non-conservative substitutions which fall under the scope of the present invention are those which still constitute a peptide having anti-bacterial properties.

The N and C termini of the peptides and compositions of the present invention may be protected by function groups. Suitable functional groups are described in Green and Wuts, “Protecting Groups in Organic Synthesis”, John Wiley and Sons, Chapters 5 and 7, 1991, the teachings of which are incorporated herein by reference. Preferred protecting groups are those that facilitate transport of the compound attached thereto into a cell, for example, by reducing the hydrophilicity and increasing the lipophilicity of the compounds.

According to specific embodiments, one or more of the amino acids may be modified by the addition of a functional group, for example (conceptually views as “chemically modified”). For example, the side amino acid residues appearing in the native sequence may optionally be modified, although as described below alternatively other parts of the protein may optionally be modified, in addition to or in place of the side amino acid residues. The modification may optionally be performed during synthesis of the molecule if a chemical synthetic process is followed, for example by adding a chemically modified amino acid. However, chemical modification of an amino acid when it is already present in the molecule (“in situ” modification) is also possible. Modifications to the peptide or protein can be introduced by gene synthesis, site-directed (e.g., PCR based) or random mutagenesis (e.g., EMS) by exonuclease deletion, by chemical modification, or by fusion of polynucleotide sequences encoding a heterologous domain or binding protein, for example.

As used herein the term “chemical modification”, when referring to a peptide, refers to a peptide where at least one of its amino acid residues is modified either by natural processes, such as processing or other post-translational modifications, or by chemical modification techniques which are well known in the art. Non-limiting exemplary types of modification include carboxymethylation, acetylation, acylation, phosphorylation, glycosylation, amidation, ADP-ribosylation, fatty acylation, addition of farnesyl group, an isofarnesyl group, a carbohydrate group, a fatty acid group, a linker for conjugation, functionalization, GPI anchor formation, covalent attachment of a lipid or lipid derivative, methylation, myristylation, pegylation, prenylation, phosphorylation, ubiquitination, or any similar process and known protecting/blocking groups. Ether bonds can optionally be used to join the serine or threonine hydroxyl to the hydroxyl of a sugar. Amide bonds can optionally be used to join the glutamate or aspartate carboxyl groups to an amino group on a sugar (Garg and Jeanloz, Advances in Carbohydrate Chemistry and Biochemistry, Vol. 43, Academic Press (1985); Kunz, Ang. Chem. Int. Ed. English 26:294-308 (1987)). Acetal and ketal bonds can also optionally be formed between amino acids and carbohydrates. Fatty acid acyl derivatives can optionally be made, for example, by acylation of a free amino group (e.g., lysine) (Toth et al., Peptides: Chemistry, Structure and Biology, Rivier and Marshal, eds., ESCOM Publ., Leiden, 1078-1079 (1990)).

According to specific embodiments, the modifications include the addition of a cycloalkane moiety to the peptide, as described in PCT Application No. WO 2006/050262, hereby incorporated by reference as if fully set forth herein. These moieties are designed for use with biomolecules and may optionally be used to impart various properties to proteins.

Furthermore, optionally any point on the peptide may be modified. For example, pegylation of a glycosylation moiety on a protein may optionally be performed, as described in PCT Application No. WO 2006/050247, hereby incorporated by reference as if fully set forth herein, and as further described hereinabove. One or more polyethylene glycol (PEG) groups may optionally be added to O-linked and/or N-linked glycosylation. The PEG group may optionally be branched or linear. Optionally any type of water-soluble polymer may be attached to a glycosylation site on a protein through a glycosyl linker.

According to specific embodiments, the peptide is modified to have an altered glycosylation pattern (i.e., altered from the original or native glycosylation pattern). As used herein, “altered” means having one or more carbohydrate moieties deleted, and/or having at least one glycosylation site added to the original protein.

Glycosylation of proteins is typically either N-linked or O-linked. N-linked refers to the attachment of the carbohydrate moiety to the side chain of an asparagine residue. The tripeptide sequences, asparagine-X-serine and asparagine-X-threonine, where X is any amino acid except proline, are the recognition sequences for enzymatic attachment of the carbohydrate moiety to the asparagine side chain. Thus, the presence of either of these tripeptide sequences in a polypeptide creates a potential glycosylation site. O-linked glycosylation refers to the attachment of one of the sugars N-acetylgalactosamine, galactose, or xylose to a hydroxyamino acid, most commonly serine or threonine, although 5-hydroxyproline or 5-hydroxylysine may also be used.

Addition of glycosylation sites to a peptide is conveniently accomplished by altering the amino acid sequence of the peptide such that it contains one or more of the above-described tripeptide sequences (for N-linked glycosylation sites). The alteration may also be made by the addition of, or substitution by, one or more serine or threonine residues in the sequence of the original peptide (for O-linked glycosylation sites). The peptide's amino acid sequence may also be altered by introducing changes at the DNA level.

Another means of increasing the number of carbohydrate moieties on peptides is by chemical or enzymatic coupling of glycosides to the amino acid residues of the peptide. Depending on the coupling mode used, the sugars may be attached to (a) arginine and histidine, (b) free carboxyl groups, (c) free sulfhydryl groups such as those of cysteine, (d) free hydroxyl groups such as those of serine, threonine, or hydroxyproline, (e) aromatic residues such as those of phenylalanine, tyrosine, or tryptophan, or (f) the amide group of glutamine. These methods are described e.g. in WO 87/05330, and in Aplin and Wriston, CRC Crit. Rev. Biochem., 22:259-306 (1981).

Removal of any carbohydrate moieties present on a peptide may be accomplished chemically, enzymatically or by introducing changes at the DNA level. Chemical deglycosylation requires exposure of the peptide to trifluoromethanesulfonic acid, or an equivalent compound. This treatment results in the cleavage of most or all sugars except the linking sugar (N-acetylglucosamine or N-acetylgalactosamine), leaving the amino acid sequence intact.

Chemical deglycosylation is described by Hakimuddin et al., Arch. Biochem. Biophys., 259:52 (1987); and Edge et al., Anal. Biochem., 118:131 (1981). Enzymatic cleavage of carbohydrate moieties on peptides can be achieved by the use of a variety of endo- and exo-glycosidases as described by Thotakura et al., Meth. Enzymol., 138:350 (1987).

The compositions [e.g. LILRB (e.g. LILRB2, LILRB1) polypeptides, compositions, fusions or dimers comprising same] of some embodiments of the invention may be synthesized and purified by any techniques that are known to those skilled in the art of peptide synthesis, such as, but not limited to, solid phase and recombinant techniques.

According to specific embodiments, preparing the polypeptide involves solid phase peptide synthesis.

For solid phase peptide synthesis, a summary of the many techniques may be found in J. M. Stewart and J. D. Young, Solid Phase Peptide Synthesis, W. H. Freeman Co. (San Francisco), 1963 and J. Meienhofer, Hormonal Proteins and Peptides, vol. 2, p. 46, Academic Press (New York), 1973. For classical solution synthesis see G. Schroder and K. Lupke, The Peptides, vol. 1, Academic Press (New York), 1965.

In general, these methods comprise the sequential addition of one or more amino acids or suitably protected amino acids to a growing peptide chain. Normally, either the amino or carboxyl group of the first amino acid is protected by a suitable protecting group. The protected or derivatized amino acid can then either be attached to an inert solid support or utilized in solution by adding the next amino acid in the sequence having the complimentary (amino or carboxyl) group suitably protected, under conditions suitable for forming the amide linkage. The protecting group is then removed from this newly added amino acid residue and the next amino acid (suitably protected) is then added, and so forth. After all the desired amino acids have been linked in the proper sequence, any remaining protecting groups (and any solid support) are removed sequentially or concurrently, to afford the final peptide compound. By simple modification of this general procedure, it is possible to add more than one amino acid at a time to a growing chain, for example, by coupling (under conditions which do not racemize chiral centers) a protected tripeptide with a properly protected dipeptide to form, after deprotection, a pentapeptide and so forth. Further description of peptide synthesis is disclosed in U.S. Pat. No. 6,472,505.

Large scale peptide synthesis is described by Andersson Biopolymers 2000; 55 (3): 227-50.

According to specific embodiments, the polypeptide [e.g. LILRB (e.g. LILRB2, LILRB1) polypeptides, compositions, fusions or dimers comprising same] is synthesized using in vitro expression systems.

Hence, any of the polypeptides described herein can be encoded from a polynucleotide. These polynucleotides can be used per se or in the recombinant production of the polypeptides disclosed herein.

A “recombinant” polypeptide refers to a polypeptide produced by recombinant DNA techniques; i.e., produced from cells transformed by an exogenous DNA construct encoding the desired polypeptide.

Thus, according to another aspect of the present invention, there is provided a polynucleotide encoding the LILRB (e.g. LILRB2, LILRB1) polypeptide, the fusion polypeptide or the dimer disclosed herein.

Non-limiting examples of polynucleotide sequences which may be used with specific embodiments of the invention are described in Table 3 hereinbelow.

Non-limiting examples of polynucleotides encoding the LILRB2 polypeptide of some embodiments of the invention are provided in SEQ ID NO: 6, 8, 10, 12, 14, 16, 18, 20, 22 and 24.

Non-limiting examples of polynucleotides encoding a LILRB2-Fc fusion polypeptide of some embodiments of the invention are provided in SEQ ID NO: 40, 42, 44, 46, 48, 50, 52, 54, 56, 58, 97 and 106.

Non-limiting examples of polynucleotides encoding a a SIRPα-Fc fusion polypeptide of some embodiments of the invention is provided in SEQ ID NO: 36, 91, 95 and 99.

As used herein the term “polynucleotide” refers to a single or double stranded nucleic acid sequence which is isolated and provided in the form of an RNA sequence, a complementary polynucleotide sequence (cDNA), a genomic polynucleotide sequence and/or a composite polynucleotide sequences (e.g., a combination of the above).

According to specific embodiments, any of the polynucleotides and nucleic acid sequences disclosed herein may comprise conservative nucleic acid substitutions. Conservatively modified polynucleotides refer to those nucleic acids which encode identical or essentially identical amino acid sequences, or where the nucleic acid does not encode an amino acid sequence, to essentially identical or associated (e.g., naturally contiguous) sequences. Because of the degeneracy of the genetic code, a large number of functionally identical nucleic acids encode most proteins. For instance, the codons GCA, GCC, GCG and GCU all encode the amino acid alanine. Thus, at every position where an alanine is specified by a codon, the codon can be altered to another of the corresponding codons described without altering the encoded polypeptide. Such nucleic acid variations are “silent variations”, which are one species of conservatively modified polynucleotides. According to specific embodiments, any polynucleotide and nucleic acid sequence described herein which encodes a polypeptide also describes silent variations of the nucleic acid. One of skill will recognize that in certain contexts each codon in a nucleic acid (except AUG, which is ordinarily the only codon for methionine, and TGG, which is ordinarily the only codon for tryptophan) can be modified to yield a functionally identical molecule. Accordingly, silent variations of a polynucleotide which encodes a polypeptide is implicit in a described sequence with respect to the expression product.

To express an exogenous polypeptide in mammalian cells, a polynucleotide sequence encoding the polypeptide is preferably ligated into a nucleic acid construct suitable for mammalian cell expression. Such a nucleic acid construct includes a promoter sequence for directing transcription of the polynucleotide sequence in the cell in a constitutive or inducible manner.

Thus, according to an aspect of the present invention there is provided a nucleic acid construct comprising a polynucleotide encoding the LILRB2 polypeptide, the fusion polypeptide or the dimer, and a regulatory element for directing expression of said polynucleotide in a host cell.

According to specific embodiments, the regulatory element (promoter) is a heterologous regulatory element.

The nucleic acid construct (also referred to herein as an “expression vector”) of some embodiments of the invention includes additional sequences which render this vector suitable for replication and integration in prokaryotes, eukaryotes, or preferably both (e.g., shuttle vectors). In addition, a typical cloning vector may also contain a transcription and translation initiation sequence, transcription and translation terminator and a polyadenylation signal. By way of example, such constructs will typically include a 5′ LTR, a tRNA binding site, a packaging signal, an origin of second-strand DNA synthesis, and a 3′ LTR or a portion thereof.

The nucleic acid construct of some embodiments of the invention typically includes a signal sequence for secretion of the peptide from a host cell in which it is placed. Preferably the signal sequence for this purpose is a mammalian signal sequence or the signal sequence of the polypeptide variants of some embodiments of the invention. According to specific embodiments, the nucleic acid construct comprises a signal peptide, such as provided in e.g. SEQ ID NO: 25-26.

Eukaryotic promoters typically contain two types of recognition sequences, the TATA box and upstream promoter elements. The TATA box, located 25-30 base pairs upstream of the transcription initiation site, is thought to be involved in directing RNA polymerase to begin RNA synthesis. The other upstream promoter elements determine the rate at which transcription is initiated.

Preferably, the promoter utilized by the nucleic acid construct of some embodiments of the invention is active in the specific cell population transformed. Examples of cell type-specific and/or tissue-specific promoters include promoters such as albumin that is liver specific [Pinkert et al., (1987) Genes Dev. 1:268-277], lymphoid specific promoters [Calame et al., (1988) Adv. Immunol. 43:235-275]; in particular promoters of T-cell receptors [Winoto et al., (1989) EMBO J. 8:729-733] and immunoglobulins; [Banerji et al. (1983) Cell 33729-740], neuron-specific promoters such as the neurofilament promoter [Byrne et al. (1989) Proc. Natl. Acad. Sci. USA 86:5473-5477], pancreas-specific promoters [Edlunch et al. (1985) Science 230:912-916] or mammary gland-specific promoters such as the milk whey promoter (U.S. Pat. No. 4,873,316 and European Application Publication No. 264,166).

Enhancer elements can stimulate transcription up to 1,000 fold from linked homologous or heterologous promoters. Enhancers are active when placed downstream or upstream from the transcription initiation site. Many enhancer elements derived from viruses have a broad host range and are active in a variety of tissues. For example, the SV40 early gene enhancer is suitable for many cell types. Other enhancer/promoter combinations that are suitable for some embodiments of the invention include those derived from polyoma virus, human or murine cytomegalovirus (CMV), the long term repeat from various retroviruses such as murine leukemia virus, murine or Rous sarcoma virus and HIV. See, Enhancers and Eukaryotic Expression, Cold Spring Harbor Press, Cold Spring Harbor, N.Y. 1983, which is incorporated herein by reference. In the construction of the expression vector, the promoter is preferably positioned approximately the same distance from the heterologous transcription start site as it is from the transcription start site in its natural setting. As is known in the art, however, some variation in this distance can be accommodated without loss of promoter function.

Polyadenylation sequences can also be added to the expression vector in order to increase the efficiency of mRNA translation. Two distinct sequence elements are required for accurate and efficient polyadenylation: GU or U rich sequences located downstream from the polyadenylation site and a highly conserved sequence of six nucleotides, AAUAAA, located 11-nucleotides upstream. Termination and polyadenylation signals that are suitable for some embodiments of the invention include those derived from SV40.

In addition to the elements already described, the expression vector of some embodiments of the invention may typically contain other specialized elements intended to increase the level of expression of cloned nucleic acids or to facilitate the identification of cells that carry the recombinant DNA. For example, a number of animal viruses contain DNA sequences that promote the extra chromosomal replication of the viral genome in permissive cell types. Plasmids bearing these viral replicons are replicated episomally as long as the appropriate factors are provided by genes either carried on the plasmid or with the genome of the host cell.

The vector may or may not include a eukaryotic replicon. If a eukaryotic replicon is present, then the vector is amplifiable in eukaryotic cells using the appropriate selectable marker. If the vector does not comprise a eukaryotic replicon, no episomal amplification is possible. Instead, the recombinant DNA integrates into the genome of the engineered cell, where the promoter directs expression of the desired nucleic acid.

The expression vector of some embodiments of the invention can further include additional polynucleotide sequences that allow, for example, the translation of several proteins from a single mRNA such as an internal ribosome entry site (IRES) and sequences for genomic integration of the promoter-chimeric polypeptide.

Thus, according to specific embodiments, both monomers comprised in the heterodimer are expressed from a single construct.

According to other specific embodiments, each of the monomers comprised in the heterodimer is expressed from a different construct.

It will be appreciated that the individual elements comprised in the expression vector can be arranged in a variety of configurations. For example, enhancer elements, promoters and the like, and even the polynucleotide sequence(s) encoding the polypeptide arranged in a “head-to-tail” configuration, may be present as an inverted complement, or in a complementary configuration, as an anti-parallel strand. While such variety of configuration is more likely to occur with non-coding elements of the expression vector, alternative configurations of the coding sequence within the expression vector are also envisioned.

Examples for mammalian expression vectors include, but are not limited to, pcDNA3, pcDNA3.1 (+/−), pcDNA3.4, pGL3, pZcoSV2 (+/−), pSecTag2, pDisplay, pEF/myc/cyto, pCMV/myc/cyto, pCR.3.1, pSinRep5, DH26S, DHBB, pNMT1, pNMT41, pNMT81, which are available from Invitrogen, pCI which is available from Promega, pMbac, pPbac, pBK-RSV and pBK-CMV which are available from Strategene, pTRES which is available from Clontech, and their derivatives.

Expression vectors containing regulatory elements from eukaryotic viruses such as retroviruses can be also used. SV40 vectors include pSVT7 and pMT2. Vectors derived from bovine papilloma virus include pBV-IMTHA, and vectors derived from Epstein Bar virus include pHEBO, and p2O5. Other exemplary vectors include pMSG, pAV009/A+, pMTO10/A+, pMAMneo-5, baculovirus pDSVE, and any other vector allowing expression of proteins under the direction of the SV-40 early promoter, SV-40 later promoter, metallothioncin promoter, murine mammary tumor virus promoter, Rous sarcoma virus promoter, polyhedrin promoter, or other promoters shown effective for expression in eukaryotic cells.

As described above, viruses are very specialized infectious agents that have evolved, in many cases, to elude host defense mechanisms. Typically, viruses infect and propagate in specific cell types. The targeting specificity of viral vectors utilizes its natural specificity to specifically target predetermined cell types and thereby introduce a recombinant gene into the infected cell. Thus, the type of vector used by some embodiments of the invention will depend on the cell type transformed. The ability to select suitable vectors according to the cell type transformed is well within the capabilities of the ordinary skilled artisan and as such no general description of selection consideration is provided herein. For example, bone marrow cells can be targeted using the human T cell leukemia virus type I (HTLV-I) and kidney cells may be targeted using the heterologous promoter present in the baculovirus Autographa californica nucleopolyhedrovirus (AcMNPV) as described in Liang C Y et al., 2004 (Arch Virol. I49:51-60).

Recombinant viral vectors are useful for in vivo expression of the polypeptide since they offer advantages such as lateral infection and targeting specificity. Lateral infection is inherent in the life cycle of, for example, retrovirus and is the process by which a single infected cell produces many progeny virions that bud off and infect neighboring cells. The result is that a large area becomes rapidly infected, most of which was not initially infected by the original viral particles. This is in contrast to vertical-type of infection in which the infectious agent spreads only through daughter progeny. Viral vectors can also be produced that are unable to spread laterally. This characteristic can be useful if the desired purpose is to introduce a specified gene into only a localized number of targeted cells.

Various methods can be used to introduce the expression vector of some embodiments of the invention into cells. Such methods are generally described in Sambrook et al., Molecular Cloning: A Laboratory Manual, Cold Springs Harbor Laboratory, New York (1989, 1992), in Ausubel et al., Current Protocols in Molecular Biology, John Wiley and Sons, Baltimore, Md. (1989), Chang et al., Somatic Gene Therapy, CRC Press, Ann Arbor, Mich. (1995), Vega et al., Gene Targeting, CRC Press, Ann Arbor Mich. (1995), Vectors: A Survey of Molecular Cloning Vectors and Their Uses, Butterworths, Boston Mass. (1988) and Gilboa et at. [Biotechniques 4 (6): 504-512, 1986] and include, for example, stable or transient transfection, Polyethylenimine lipofection, electroporation and infection with recombinant viral vectors. In addition, see U.S. Pat. Nos. 5,464,764 and 5,487,992 for positive-negative selection methods.

Introduction of nucleic acids by viral infection offers several advantages over other methods such as lipofection and electroporation, since higher transfection efficiency can be obtained due to the infectious nature of viruses.

Currently preferred in vivo nucleic acid transfer techniques include transfection with viral or non-viral constructs, such as adenovirus, lentivirus, Herpes simplex I virus, or adeno-associated virus (AAV) and lipid-based systems. Useful lipids for lipid-mediated transfer of the gene are, for example, DOTMA, DOPE, and DC-Chol [Tonkinson et al., Cancer Investigation, 14 (1): 54-65 (1996)]. The most preferred constructs for use in gene therapy are viruses, most preferably adenoviruses, AAV, lentiviruses, or retroviruses. A viral construct such as a retroviral construct includes at least one transcriptional promoter/enhancer or locus-defining element(s), or other elements that control gene expression by other means such as alternate splicing, nuclear RNA export, or post-translational modification of messenger. Such vector constructs also include a packaging signal, long terminal repeats (LTRs) or portions thereof, and positive and negative strand primer binding sites appropriate to the virus used, unless it is already present in the viral construct. In addition, such a construct typically includes a signal sequence for secretion of the peptide from a host cell in which it is placed. Preferably the signal sequence for this purpose is a mammalian signal sequence or the signal sequence of the polypeptide variants of some embodiments of the invention. Optionally, the construct may also include a signal that directs polyadenylation, as well as one or more restriction sites and a translation termination sequence. By way of example, such constructs will typically include a 5′ LTR, a tRNA binding site, a packaging signal, an origin of second-strand DNA synthesis, and a 3′ LTR or a portion thereof. Other vectors can be used that are non-viral, such as cationic lipids, polylysine, and dendrimers.

As mentioned, other than containing the necessary elements for the transcription and translation of the inserted coding sequence, the expression construct of some embodiments of the invention can also include sequences engineered to enhance stability, production, purification, yield or toxicity of the expressed polypeptide. For example, the expression of a fusion protein or a cleavable fusion protein comprising the polypeptide of some embodiments of the invention and a heterologous protein can be engineered. Such a fusion protein can be designed so that the fusion protein can be readily isolated by affinity chromatography; e.g., by immobilization on a column specific for the heterologous protein. Where a cleavage site is engineered between the polypeptide of some embodiments of the present invention and the heterologous protein, the polypeptide can be released from the chromatographic column by treatment with an appropriate enzyme or agent that disrupts the cleavage site [e.g., see Booth et al. (1988) Immunol. Lett. 19:65-70; and Gardella et al., (1990) J. Biol. Chem. 265:15854-15859].

The present invention also contemplates cells comprising the composition described herein and method of generating and using same.

Thus, according to an aspect of the present invention, there is provided a host cell comprising the LILRB (e.g. LILRB2, LILRB1) polypeptide, the fusion polypeptide or the dimer comprising same, or the polynucleotide encoding same.

As mentioned hereinabove, a variety of prokaryotic or eukaryotic cells can be used as host-expression systems to express the polypeptide of some embodiments of the invention. These include, but are not limited to, microorganisms, such as bacteria transformed with a recombinant bacteriophage DNA, plasmid DNA or cosmid DNA expression vector containing the coding sequence; yeast transformed with recombinant yeast expression vectors containing the coding sequence; plant cell systems infected with recombinant virus expression vectors (e.g., cauliflower mosaic virus, CaMV; tobacco mosaic virus, TMV) or transformed with recombinant plasmid expression vectors, such as Ti plasmid, containing the coding sequence. Mammalian expression systems can also be used to express the polypeptides of some embodiments of the invention.

Examples of bacterial constructs include the pET series of E. coli expression vectors [Studier et al. (1990) Methods in Enzymol. 185:60-89).

Examples of eukaryotic cells which may be used along with the teachings of the invention include but are not limited to, mammalian cells, fungal cells, yeast cells, insect cells, algal cells or plant cells.

In yeast, a number of vectors containing constitutive or inducible promoters can be used, as disclosed in U.S. Pat. No. 5,932,447. Alternatively, vectors can be used which promote integration of foreign DNA sequences into the yeast chromosome.

In cases where plant expression vectors are used, the expression of the coding sequence can be driven by a number of promoters. For example, viral promoters such as the 35S RNA and 19S RNA promoters of CaMV [Brisson et al. (1984) Nature 310:511-514], or the coat protein promoter to TMV [Takamatsu et al. (1987) EMBO J. 6:307-311] can be used. Alternatively, plant promoters such as the small subunit of RUBISCO [Coruzzi et al. (1984) EMBO J. 3:1671-1680 and Brogli et al., (1984) Science 224:838-843] or heat shock promoters, e.g., soybean hsp17.5-E or hsp17.3-B [Gurley et al. (1986) Mol. Cell. Biol. 6:559-565] can be used. These constructs can be introduced into plant cells using Ti plasmid, Ri plasmid, plant viral vectors, direct DNA transformation, microinjection, electroporation and other techniques well known to the skilled artisan. See, for example, Weissbach & Weissbach, 1988, Methods for Plant Molecular Biology, Academic Press, NY, Section VIII, pp 421-463.

Other expression systems such as insects and mammalian host cell systems which are well known in the art can also be used by some embodiments of the invention.

According to specific embodiments the cell is a mammalian cell.

According to specific embodiment, the cell is a human cell.

According to specific embodiments, the cell is not derived from a human embryo.

According to specific embodiments, the cell is an isolated cell.

According to a specific embodiment, the cell is a cell line.

According to another specific embodiment, the cell is a primary cell.

The cell may be derived from a suitable tissue including but not limited to blood, muscle, nerve, brain, heart, lung, liver, pancreas, spleen, thymus, esophagus, stomach, intestine, kidney, testis, ovary, hair, skin, bone, breast, uterus, bladder, spinal cord, or various kinds of body fluids. The cells may be derived from any developmental stage including embryo, fetal and adult stages, as well as developmental origin i.e., ectodermal, mesodermal, and endodermal origin.

Non limiting examples of mammalian cells include monkey kidney CV1 line transformed by SV40 (COS, e.g. COS-7, ATCC CRL 1651); human embryonic kidney line (HEK293 or HEK293 cells subcloned for growth in suspension culture, Graham et al., J. Gen Virol., 36:59 1977); baby hamster kidney cells (BHK, ATCC CCL 10); mouse sertoli cells (TM4, Mather, Biol. Reprod., 23:243-251 1980); monkey kidney cells (CV1 ATCC CCL 70); African green monkey kidney cells (VERO-76, ATCC CRL-1587); human cervical carcinoma cells (HeLa, ATCC CCL 2); NIH3T3, Jurkat, canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TRI cells (Mather et al., Annals N.Y. Acad. Sci., 383:44-68 1982); MRC 5 cells; FS4 cells; and a human hepatoma line (Hep G2), PER.C6, K562, and Chinese hamster ovary cells (CHO).

According to some embodiments of the invention, the mammalian cell is selected from the group consisting of a Chinese Hamster Ovary (CHO), HEK293, PER.C6, HT1080, NSO, Sp2/0, BHK, Namalwa, COS, HeLa and Vero cell.

According to some embodiments of the invention, the host cell comprises a Chinese Hamster Ovary (CHO), PER.C6 or a 293 (e.g. Expi293F) cell.

According to another aspect of the present invention, there is provided method of producing a polypeptide, the method comprising introducing the polynucleotide of nucleic acid construct described herein to a host cell or culturing the cells expressing the polynucleotide or nucleic acid construct described herein.

According to specific embodiments, the method is an in-vitro or an ex-vivo method.

According to specific embodiments, the producing comprises culturing at 32-37° C., 5-10% CO₂ for 5-13 days.

Non-limiting examples of production conditions that can be used with specific embodiments of the invention are disclosed in the Examples section which follows.

Thus, for example an expression vector encoding the polypeptide, is introduced into mammalian cells such as Expi293F, ExpiCHO cells, CHO-K1, CHO—S, CHO-DUC or CHO-DG44. The transfected cells are then cultured at 32-37° C. 5-10% CO₂ in cell-specific culture medium and following at least 5 days in culture the proteins are collected from the supernatant and purified.

According to specific embodiments the culture is operated in a batch, split-batch, fed-batch, or perfusion mode.

According to specific embodiments, the culture is operated under fed-batch conditions.

According to specific embodiments, the culturing is effected at 36.5° C.

According to specific embodiments, the culturing it effected at 36.5° C. with a temperature shift to 32° C. This temperature shift can be effected to slow down cells metabolism prior to reaching a stationary phase.

According to specific embodiments, the methods comprising isolating the LILRB (e.g. LILRB2, LILRB1) polypeptide, the fusion polypeptide or the dimer.

According to specific embodiments, recovery of the recombinant the polypeptide is effected following an appropriate time in culture.

According to specific embodiments, recovering the recombinant the polypeptide refers to collecting the whole culture medium containing the heterodimer and need not imply additional steps of separation or purification. According to specific embodiments, the polypeptide of some embodiments of the invention can be purified using a variety of standard protein purification techniques, such as, but not limited to, affinity chromatography, ion exchange chromatography, filtration, electrophoresis, hydrophobic interaction chromatography, gel filtration chromatography, reverse phase chromatography, concanavalin A chromatography, mix mode chromatography, metal affinity chromatography, Lectins affinity chromatography chromatofocusing and differential solubilization.

According to specific embodiments, following production and purification, the therapeutic efficacy of the polypeptide can be assayed either in vivo or in vitro. Such methods are known in the art and include for example binding, cell viability, survival of transgenic mice, and expression of activation markers.

The compositions [e.g. the LILRB (e.g. LILRB2, LILRB1) polypeptide, composition, fusion protein or dimer comprising same, polynucleotide encoding same and/or cells described herein] of some embodiments of the invention can be administered to an organism per se, or in a pharmaceutical composition where it is mixed with suitable carriers or excipients.

Thus, the present invention, in some embodiments, features a pharmaceutical composition comprising a therapeutically effective amount of the composition disclosed herein.

Herein the term “active ingredient” refers to the composition [e.g. the LILRB (e.g. LILRB2, LILRB1) polypeptide, composition, fusion protein or dimer comprising same, polynucleotide encoding same and/or cells described herein] accountable for the biological effect.

Herein the term “excipient” refers to an inert substance added to a pharmaceutical composition to further facilitate administration of an active ingredient. Examples, without limitation, of excipients include calcium carbonate, calcium phosphate, various sugars and types of starch, cellulose derivatives, gelatin, vegetable oils and polyethylene glycols.

Hereinafter, the phrases “physiologically acceptable carrier” and “pharmaceutically acceptable carrier” which may be interchangeably used refer to a carrier or a diluent that does not cause significant irritation to an organism and does not abrogate the biological activity and properties of the administered compound. An adjuvant is included under these phrases.

As used herein, “pharmaceutically acceptable carrier” includes any and all solvents, dispersion media, coatings, antibacterial and antifungal agents, isotonic and absorption delaying agents, and the like that are physiologically compatible. Preferably, the carrier is suitable for intravenous, intramuscular, subcutaneous, parenteral, spinal or epidermal administration (e.g., by injection or infusion). Depending on the route of administration, the active compound may include one or more pharmaceutically acceptable salts. A “pharmaceutically acceptable salt” refers to a salt that retains the desired biological activity of the parent compound and does not impart any undesired toxicological effects (see e.g., Berge, S. M., et al. (1977) J. Pharm. Sci. 66:1-19). Examples of such salts include acid addition salts and base addition salts. Acid addition salts include those derived from nontoxic inorganic acids, such as hydrochloric, nitric, phosphoric, sulfuric, hydrobromic, hydroiodic, phosphorous and the like, as well as from nontoxic organic acids such as aliphatic mono- and dicarboxylic acids, phenyl-substituted alkanoic acids, hydroxy alkanoic acids, aromatic acids, aliphatic and aromatic sulfonic acids and the like. Base addition salts include those derived from alkaline earth metals, such as sodium, potassium, magnesium, calcium and the like, as well as from nontoxic organic amines, such as N,N′-dibenzylethylenediamine, N-methylglucamine, chloroprocaine, choline, diethanolamine, ethylenediamine, procaine and the like.

A pharmaceutical composition according to at least some embodiments of the present invention also may include a pharmaceutically acceptable anti-oxidants. Examples of pharmaceutically acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like. A pharmaceutical composition according to at least some embodiments of the present invention also may include additives such as detergents and solubilizing agents (e.g., TWEEN 20 (polysorbate-20), TWEEN 80 (polysorbate-80)) and preservatives (e.g., Thimersol, benzyl alcohol) and bulking substances (e.g., lactose, mannitol).

Examples of suitable aqueous and nonaqueous carriers that may be employed in the pharmaceutical compositions according to at least some embodiments of the present invention include water, buffered saline of various buffer content (e.g., Tris-HCl, acetate, phosphate), pH and ionic strength, ethanol, polyols (such as glycerol, propylene glycol, polyethylene glycol, and the like), and suitable mixtures thereof, vegetable oils, such as olive oil, and injectable organic esters, such as ethyl oleate.

Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of presence of microorganisms may be ensured both by sterilization procedures, supra, and by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

Pharmaceutically acceptable carriers include sterile aqueous solutions or dispersions and sterile powders for the extemporaneous preparation of sterile injectable solutions or dispersion. The use of such media and agents for pharmaceutically active substances is known in the art. Except insofar as any conventional media or agent is incompatible with the active compound, use thereof in the pharmaceutical compositions according to at least some embodiments of the present invention is contemplated. Supplementary active compounds can also be incorporated into the compositions.

Therapeutic compositions typically must be sterile and stable under the conditions of manufacture and storage. The composition can be formulated as a solution, microemulsion, liposome, or other ordered structure suitable to high drug concentration. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. In many cases, it will be preferable to include isotonic agents, for example, sugars, polyalcohols such as mannitol, sorbitol, or sodium chloride in the composition. Prolonged absorption of the injectable compositions can be brought about by including in the composition an agent that delays absorption, for example, monostearate salts and gelatin. Sterile injectable solutions can be prepared by incorporating the active compound in the required amount in an appropriate solvent with one or a combination of ingredients enumerated above, as required, followed by sterilization microfiltration. Generally, dispersions are prepared by incorporating the active compound into a sterile vehicle that contains a basic dispersion medium and the required other ingredients from those enumerated above. In the case of sterile powders for the preparation of sterile injectable solutions, the preferred methods of preparation are vacuum drying and freeze-drying (lyophilization) that yield a powder of the active ingredient plus any additional desired ingredient from a previously sterile-filtered solution thereof.

Sterile injectable solutions can be prepared by incorporating the active compound in the required amount in an appropriate solvent with one or a combination of ingredients enumerated above, as required, followed by sterilization microfiltration. Generally, dispersions are prepared by incorporating the active compound into a sterile vehicle that contains a basic dispersion medium and the required other ingredients from those enumerated above. In the case of sterile powders for the preparation of sterile injectable solutions, the preferred methods of preparation are vacuum drying and freeze-drying (lyophilization) that yield a powder of the active ingredient plus any additional desired ingredient from a previously sterile-filtered solution thereof.

The amount of active ingredient which can be combined with a carrier material to produce a single dosage form will vary depending upon the subject being treated, and the particular mode of administration. The amount of active ingredient which can be combined with a carrier material to produce a single dosage form will generally be that amount of the composition which produces a therapeutic effect. Generally, out of one hundred percent, this amount will range from about 0.01 percent to about ninety-nine percent of active ingredient, preferably from about 0.1 percent to about 70 percent, most preferably from about 1 percent to about 30 percent of active ingredient in combination with a pharmaceutically acceptable carrier.

Dosage regimens are adjusted to provide the optimum desired response (e.g., a therapeutic response). For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the subjects to be treated; each unit contains a predetermined quantity of active compound calculated to produce the desired therapeutic effect in association with the required pharmaceutical carrier. The specification for the dosage unit forms according to at least some embodiments of the present invention are dictated by and directly dependent on (a) the unique characteristics of the active compound and the particular therapeutic effect to be achieved, and (b) the limitations inherent in the art of compounding such an active compound for the treatment of sensitivity in individuals.

Techniques for formulation and administration of drugs may be found in “Remington's Pharmaceutical Sciences,” Mack Publishing Co., Easton, PA, latest edition, which is incorporated herein by reference.

Pharmaceutical compositions of some embodiments of the invention may be manufactured by processes well known in the art, e.g., by means of conventional mixing, dissolving, granulating, dragee-making, levigating, emulsifying, encapsulating, entrapping or lyophilizing processes.

A composition of the present invention can be administered via one or more routes of administration using one or more of a variety of methods known in the art. As will be appreciated by the skilled artisan, the route and/or mode of administration will vary depending upon the desired results. Preferred routes of administration for therapeutic agents according to at least some embodiments of the present invention include intravascular delivery (e.g. injection or infusion), intravenous, intramuscular, intradermal, intraperitoneal, subcutaneous, spinal, oral, enteral, rectal, pulmonary (e.g. inhalation), nasal, topical (including transdermal, buccal and sublingual), intravesical, intravitreal, intraperitoneal, vaginal, brain delivery (e.g. intra-cerebroventricular, intra-cerebral, and convection enhanced diffusion), CNS delivery (e.g. intrathecal, perispinal, and intra-spinal) or parenteral (including subcutaneous, intramuscular, intraperitoneal, intravenous (IV) and intradermal), transdermal (either passively or using iontophoresis or electroporation), transmucosal (e.g., sublingual administration, nasal, vaginal, rectal, or sublingual), administration or administration via an implant, or other parenteral routes of administration, for example by injection or infusion, or other delivery routes and/or forms of administration known in the art. The phrase “parenteral administration” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion or using bioerodible inserts, and can be formulated in dosage forms appropriate for each route of administration. In a specific embodiment, a protein, a therapeutic agent or a pharmaceutical composition according to at least some embodiments of the present invention can be administered intraperitoneally or intravenously.

According to specific embodiments, the compositions disclosed herein are administered in an aqueous solution, by parenteral injection. The formulation may also be in the form of a suspension or emulsion. In general, pharmaceutical compositions for parenteral injection are provided including effective amounts of the compositions described herein, and optionally include pharmaceutically acceptable diluents, preservatives, solubilizers, emulsifiers, adjuvants and/or carriers. Such compositions optionally include one or more for the following: diluents, sterile water, buffered saline of various buffer content (e.g., Tris-HCl, acetate, phosphate), pH and ionic strength; and additives such as detergents and solubilizing agents (e.g., TWEEN 20 (polysorbate-20), TWEEN 80 (polysorbate-80)), anti-oxidants (e.g., water soluble antioxidants such as ascorbic acid, sodium metabisulfite, cysteine hydrochloride, sodium bisulfate, sodium metabisulfite, sodium sulfite; oil-soluble antioxidants, such as ascorbyl palmitate, butylated hydroxyanisole (BHA), butylated hydroxytoluene (BHT), lecithin, propyl gallate, alpha-tocopherol; and metal chelating agents, such as citric acid, ethylenediamine tetraacetic acid (EDTA), sorbitol, tartaric acid, phosphoric acid), and preservatives (e.g., Thimersol, benzyl alcohol) and bulking substances (e.g., lactose, mannitol). Examples of non-aqueous solvents or vehicles are ethanol, propylene glycol, polyethylene glycol, vegetable oils, such as olive oil and corn oil, gelatin, and injectable organic esters such as ethyl oleate. The formulations may be freeze dried (lyophilized) or vacuum dried and redissolved/resuspended immediately before use. The formulation may be sterilized by, for example, filtration through a bacteria retaining filter, by incorporating sterilizing agents into the compositions, by irradiating the compositions, or by heating the compositions.

Various compositions (e.g., polypeptides) disclosed herein can be applied topically. Topical administration does not work well for most peptide formulations, although it can be effective especially if applied to the lungs, nasal, oral (sublingual, buccal), vaginal, or rectal mucosa.

Compositions of the present invention can be delivered to the lungs while inhaling and traverse across the lung epithelial lining to the blood stream when delivered either as an aerosol or spray dried particles having an acrodynamic diameter of less than about 5 microns. A wide range of mechanical devices designed for pulmonary delivery of therapeutic products can be used, including but not limited to nebulizers, metered dose inhalers, and powder inhalers, all of which are familiar to those skilled in the art. Some specific examples of commercially available devices are the Ultravent nebulizer (Mallinckrodt Inc., St. Louis, Mo.); the Acorn II nebulizer (Marquest Medical Products, Englewood, Colo.); the Ventolin metered dose inhaler (Glaxo Inc., Research Triangle Park, N.C.); and the Spinhaler powder inhaler (Fisons Corp., Bedford, Mass.). Nektar, Alkermes and Mannkind all have inhalable insulin powder preparations approved or in clinical trials where the technology could be applied to the formulations described herein.

Formulations for administration to the mucosa will typically be spray dried drug particles, which may be incorporated into a tablet, gel, capsule, suspension or emulsion. Standard pharmaceutical excipients are available from any formulator. Oral formulations may be in the form of chewing gum, gel strips, tablets or lozenges.

Transdermal formulations may also be prepared. These will typically be ointments, lotions, sprays, or patches, all of which can be prepared using standard technology. Transdermal formulations will require the inclusion of penetration enhancers. Actual dosage levels of the active ingredients in the pharmaceutical compositions of the present invention may be varied so as to obtain an amount of the active ingredient which is effective to achieve the desired therapeutic response for a particular patient, composition, and mode of administration, without being toxic to the patient. The selected dosage level will depend upon a variety of pharmacokinetic factors including the activity of the particular compositions of the present invention employed, the route of administration, the time of administration, the rate of excretion of the particular compound being employed, the duration of the treatment, other drugs, compounds and/or materials used in combination with the particular compositions employed, the age, sex, weight, condition, general health and prior medical history of the patient being treated, and like factors well known in the medical arts.

According to specific embodiments, the compositions disclosed herein are administered to a subject in a therapeutically effective amount. As used herein the term “effective amount” or “therapeutically effective amount” means a dosage sufficient to treat, inhibit, or alleviate one or more symptoms of the disorder being treated or to otherwise provide a desired pharmacologic and/or physiologic effect. Determination of a therapeutically effective amount is well within the capability of those skilled in the art, especially in light of the detailed disclosure provided herein.

For any preparation used in the methods of the invention, the therapeutically effective amount or dose can be estimated initially from in vitro and cell culture assays. For example, a dose can be formulated in animal models to achieve a desired concentration or titer. Such information can be used to more accurately determine useful doses in humans. Toxicity and therapeutic efficacy of the active ingredients described herein can be determined by standard pharmaceutical procedures in vitro, in cell cultures or experimental animals. The data obtained from these in vitro and cell culture assays and animal studies can be used in formulating a range of dosage for use in human. The dosage may vary depending upon the dosage form employed and the route of administration utilized. The exact formulation, route of administration and dosage can be chosen by the individual physician in view of the patient's condition. (See e.g., Fingl, et al., 1975, in “The Pharmacological Basis of Therapeutics”, Ch. 1 p.1).

Dosage amount and interval may be adjusted individually to provide levels of the active ingredient are sufficient to induce or suppress the biological effect (minimal effective concentration, MEC). The MEC will vary for each preparation, but can be estimated from in vitro data. Dosages necessary to achieve the MEC will depend on individual characteristics and route of administration. Detection assays can be used to determine plasma concentrations.

Depending on the severity and responsiveness of the condition to be treated, dosing can be of a single or a plurality of administrations, with course of treatment lasting from several days to several weeks or until cure is effected or diminution of the disease state is achieved.

The amount of a composition to be administered will, of course, be dependent on the subject being treated, the severity of the affliction, the manner of administration, the judgment of the prescribing physician, etc.

In certain embodiments, the composition [e.g. the LILRB (e.g. LILRB2, LILRB1) polypeptide, composition, fusion protein or dimer comprising same, polynucleotide encoding same and/or cells described herein] is administered locally, for example by injection directly into a site to be treated. Typically, the injection causes an increased localized concentration of the composition which is greater than that which can be achieved by systemic administration. The compositions can be combined with a matrix as described above to assist in creating an increased localized concentration of the polypeptide compositions by reducing the passive diffusion of the polypeptides out of the site to be treated.

Pharmaceutical compositions of the present invention may be administered with medical devices known in the art. For example, in an optional embodiment, a pharmaceutical composition according to at least some embodiments of the present invention can be administered with a needle hypodermic injection device, such as the devices disclosed in U.S. Pat. Nos. 5,399,163; 5,383,851; 5,312,335; 5,064,413; 4,941,880; 4,790,824; or 4,596,556. Examples of well-known implants and modules useful in the present invention include: U.S. Pat. No. 4,487,603, which discloses an implantable micro-infusion pump for dispensing medication at a controlled rate; U.S. Pat. No. 4,486,194, which discloses a therapeutic device for administering medicaments through the skin; U.S. Pat. No. 4,447,233, which discloses a medication infusion pump for delivering medication at a precise infusion rate; U.S. Pat. No. 4,447,224, which discloses a variable flow implantable infusion apparatus for continuous drug delivery; U.S. Pat. No. 4,439,196, which discloses an osmotic drug delivery system having multi-chamber compartments; and U.S. Pat. No. 4,475,196, which discloses an osmotic drug delivery system. These patents are incorporated herein by reference. Many other such implants, delivery systems, and modules are known to those skilled in the art.

The active compounds can be prepared with carriers that will protect the compound against rapid release, such as a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are patented or generally known to those skilled in the art. See, e.g., Sustained and Controlled Release Drug Delivery Systems, J. R. Robinson, ed., Marcel Dekker, Inc., New York, 1978.

Controlled release polymeric devices can be made for long term release systemically following implantation of a polymeric device (rod, cylinder, film, disk) or injection (microparticles). The matrix can be in the form of microparticles such as microspheres, where peptides are dispersed within a solid polymeric matrix or microcapsules, where the core is of a different material than the polymeric shell, and the peptide is dispersed or suspended in the core, which may be liquid or solid in nature. Unless specifically defined herein, microparticles, microspheres, and microcapsules are used interchangeably. Alternatively, the polymer may be cast as a thin slab or film, ranging from nanometers to four centimeters, a powder produced by grinding or other standard techniques, or even a gel such as a hydrogel.

Either non-biodegradable or biodegradable matrices can be used for delivery of the active agents disclosed herein, although biodegradable matrices are preferred. These may be natural or synthetic polymers, although synthetic polymers are preferred due to the better characterization of degradation and release profiles. The polymer is selected based on the period over which release is desired. In some cases linear release may be most useful, although in others a pulse release or “bulk release” may provide more effective results. The polymer may be in the form of a hydrogel (typically in absorbing up to about 90% by weight of water), and can optionally be crosslinked with multivalent ions or polymers.

The matrices can be formed by solvent evaporation, spray drying, solvent extraction and other methods known to those skilled in the art. Bioerodible microspheres can be prepared using any of the methods developed for making microspheres for drug delivery, for example, as described by Mathiowitz and Langer, J. Controlled Release, 5:13-22 (1987); Mathiowitz, et al., Reactive Polymers, 6:275-283 (1987); and Mathiowitz, et al., J. Appl Polymer ScL, 35:755-774 (1988).

The devices can be formulated for local release to treat the area of implantation or injection-which will typically deliver a dosage that is much less than the dosage for treatment of an entire body- or systemic delivery. These can be implanted or injected subcutaneously, into the muscle, fat, or swallowed.

In certain embodiments, to ensure that the therapeutic compounds according to at least some embodiments of the present invention cross the BBB (if desired), they can be formulated, for example, in liposomes. For methods of manufacturing liposomes, see, e.g., U.S. Pat. Nos. 4,522,811; 5,374,548; and 5,399,331. The liposomes may comprise one or more moieties which are selectively transported into specific cells or organs, thus enhance targeted drug delivery (see, e.g., V. V. Ranade (1989) J. Clin. Pharmacol. 29:685). Exemplary targeting moieties include folate or biotin (see, e.g., U.S. Pat. No. 5,416,016 to Low et al.); mannosides (Umezawa et al., (1988) Biochem. Biophys. Res. Commun. 153:1038); antibodies (P. G. Blocman et al. (1995) FEBS Lett. 357:140; M. Owais et al. (1995) Antimicrob. Agents Chemother. 39:180); surfactant protein A receptor (Briscoe et al. (1995) Am. J Physiol. 1233:134); p120 (Schreier et al. (1994) J. Biol. Chem. 269:9090); see also K. Keinanen; M. L. Laukkanen (1994) FEBS Lett. 346:123; J. J. Killion; I. J. Fidler (1994) Immunomethods 4:273.

Compositions of some embodiments of the invention may, if desired, be presented in a pack or dispenser device, such as an FDA approved kit, which may contain one or more unit dosage forms containing the active ingredient. The pack may, for example, comprise metal or plastic foil, such as a blister pack. The pack or dispenser device may be accompanied by instructions for administration. The pack or dispenser may also be accommodated by a notice associated with the container in a form prescribed by a governmental agency regulating the manufacture, use or sale of pharmaceuticals, which notice is reflective of approval by the agency of the form of the compositions or human or veterinary administration. Such notice, for example, may be of labeling approved by the U.S. Food and Drug Administration for prescription drugs or of an approved product insert. Compositions comprising a preparation of the invention formulated in a compatible pharmaceutical carrier may also be prepared, placed in an appropriate container, and labeled for treatment of an indicated condition, as is further detailed above.

According to specific embodiments, any of the genes, polynucleotides, proteins, polypeptides and/or proteinaceous moieties described herein may have a sequence of a human gene, polynucleotide, protein, polypeptide and/or proteinaceous moiety or a functional fragment or homolog thereof which exhibit the desired activity as described herein.

According to specific embodiments, the gene, polynucleotide, protein, polypeptide and/or proteinaceous moiety is of a human origin.

According to other specific embodiments, the gene, polynucleotide, protein, polypeptide and/or proteinaceous moiety is a homolog of a human gene, polynucleotide, protein, polypeptide and/or proteinaceous moiety. Such homologues can be, for example, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identical or homologous to the human sequence.

As used herein the term “about” refers to +10%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

When reference is made to particular sequence listings, such reference is to be understood to also encompass sequences that substantially correspond to its complementary sequence as including minor sequence variations, resulting from, e.g., sequencing errors, cloning errors, or other alterations resulting in base substitution, base deletion or base addition, provided that the frequency of such variations is less than 1 in 50 nucleotides, alternatively, less than 1 in 100 nucleotides, alternatively, less than 1 in 200 nucleotides, alternatively, less than 1 in 500 nucleotides, alternatively, less than 1 in 1000 nucleotides, alternatively, less than 1 in 5,000 nucleotides, alternatively, less than 1 in 10,000 nucleotides.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non-limiting fashion.

### Example 1

### Design of LILRB2 Variants

### Materials and Methods

A structural analysis of LILRB2 (SEQ ID NO: 1, also referred to herein as “WT LILRB2”) was effected in order to identify amino acid substitutions that may optimize and increase its stability and binding affinity to HLA-G (SEQ ID NO: 3), which included the following steps:
1. Preparation of a complex structure of both LILRB2 and HLAG using the “prepare protein” protocol 1 of BioVia discovery studio (by dassault systems). The preparation including fixing charges and missing sidechains, and energy minimization.
2. Structural analysis to identify potential interacting points between LILRB2 and HLA-G (FIGS. 1A-B, PDB ID: 2DYP is the high-resolution complex structure used for this purpose).
3. Sequence comparison between HLA homologues: HLA-A, HLA-B, HLA-C and HLA-G, to identify LILRB2-interacting sites which are specific for the LILRB2-HLA-G interaction (FIG. 1B).
4. Identifying amino acids in LILRB2 interface which interact with the specific amino acid or amino acids of the HLA-G that were identified in step 3 hereinabove, i.e., not present in HLA-A, HLA-B or HLA-C (FIG. 1D).
5. In-silico-sampling each possible mutation in the subset of residues defined in steps 3-4 hereinabove on LILRB2. This process involved: computationally introducing a specific mutation at each position separately, optimizing the structure to best accommodate the mutation and using CHARMM-based force-field energy calculation to predict the energy difference (ΔG) between LILRB2 wild-type sequence (SEQ ID NO: 1) and any mutation (ΔG vs. WT) on both the entire protein stability and on the binding energy. The actual energy term that was calculated was the effect of the mutation on the binding and stability potential energy rather than ΔG only. However, ΔG is the dominant, thus the hint is on the effect on ΔG.
6. The results of the binding/stability energy calculations were compiled into a ranked list; and specific point mutations (substitutions) on LILRB2 sequence (SEQ ID NO: 1) which were predicted to be both beneficial for binding and for stability considerations were selected.
7. The selected substitutions were used in a second round of mutational analysis, this time introducing each time a combination of two substitutions and repeating the process of energy evaluation on both binding to HLA-G and stability.
   Final list of suggested substitutes for further testing were selected based on several parameters that were considered during this analysis:
The energy contribution (ΔG) of the substitutions for the stability on the apo protein (a protein devoid of its characteristic prosthetic group or metal);
The energy contribution (ΔG) of the substitutions for the stability on the complex structure and interaction energy;
Minimizing steric effects on the proteins (for example by introducing substitutions which are distant in terms of size, shape and chemical nature compared to the WT LILRB2 sequence);
Shape complementarity within the scope of the protein complex interface.

### Results

Structural analysis of HLA-G (SEQ ID NO: 3)-LILRB2 (SEQ ID NO: 1) interacting interface and sequence comparison between HLA homologues revealed only a single dominant difference between HLA-G and its homologues, namely Phe195 in HLA-G which is replaced by Ser in the other homologues (FIG. 1C). Therefore, the closely interacting residues on LILRB2 were primarily considered in the analysis.

In-silico sampling each possible mutation in a subset of amino acid residues defining the interface between LILRB2 and the HLA-G Phe195 interacting residue, namely LILRB2 (SEQ ID NO: 1) residues Ser45, Ile49, Thr50 and Val57 (FIG. 1D) were found to have a significant positive influence i.e., lower ΔG, on binding energy. In addition, the results of the calculated binding/stability energy of each possible mutation in the four amino acids, Ser45, Ile49, Thr50 and Val57, were compiled into ranked lists. Of note, ranking was effected by in-silico predicting contribution of the mutation on both the individual protein stability and the binding free energy (affinity).

Table 1 hereinbelow shows top ranked single substitutions and Table 2 hereinbelow shows top ranked double substitutions.

Following, specific single substitutions and combinations of two amino acids substitutions were selected for further analysis, as further described in the Examples which follows.

TABLE 1
Top ranked single substitutions with contributing effect

| Mutation | Mutation Energy (kcal/mol) |
|---|---|
| SER45 > GLN | −2.03 |
| SER45 > ARG | −1.75 |
| SER45 > ASN | −1.24 |
| SER45 > HIS | −2.04 |
| SER45 > LEU | −1.74 |
| SER45 > LYS | −0.76 |
| SER45 > MET | −0.82 |
| SER45 > PHE | −2.81 |
| SER45 > TRP | −3.35 |
| SER45 > TYR | −3.02 |
| ILE49 > LYS | −1.32 |
| ILE49 > ARG | −0.64 |
| ILE49 > PHE | −0.79 |
| ILE49 > TYR | −0.66 |
| THR50 > PHE | −1.64 |
| THR50 > ARG | −2.61 |
| THR50 > ASN | −1 |
| THR50 > LEU | −1.5 |
| THR50 > LYS | −1.66 |
| THR50 > TRP | −1.42 |
| THR50 > TYR | −0.93 |
| VAL57 > ARG | −0.62 |
| VAL57 > LYS | −0.34 |
| VAL57 > PHE | −0.96 |
| VAL57 > TRP | −0.53 |

TABLE 2
Top ranked double substitutions with contributing effect

| | first | second | mutation energy |
|---|---|---|---|
| Mutation | Sub | Sub | SUM (kcal/mol) |
| SER45 > ASN and THR50 > ARG | ASN N | ARG R | −8.71 |
| SER45 > TYR and THR50 > LYS | TYR Y | LYS K | −6.89 |
| SER45 > ARG and ILE49 > PHE | ARG R | PHE F | −6.74 |
| SER45 > GLN and VAL57 > ARG | GLN Q | ARG R | −6.61 |
| SER45 > GLN and ILE49 > LYS | GLN Q | LYS K | −6.34 |
| SER45 > TYR and THR50 > ASN | TYR Y | ASN N | −6.04 |

### Example 2

### Production and Characterization of LILRB2 Variants

### Materials and Methods

Reagents—ExcelBand™ 3-color high range protein marker or ExcelBand™ 3-color extra range protein marker (SMOBIO, Cat #PM2600 or PM2800 respectively), Sample buffer (GenScript Cat #M00676), Polyacrylamide gel 8% or 4-20% GenScript Cat #M00662 or M00656 respectively), FuGENE® HD Transfection Reagent (Promega, Cat #TM328), ECL Plus Western Blotting substrate (Pierce, cat #32132).

Antibodies—APC anti human HLA-G (from patent US 2020/0102390 A1), APC Human IgG4 (Bioegend, Cat #403706), APC anti human IgG1 (Southern Biotech, Cat #9052-31), APC Mouse IgG1 k (Biolegend, Cat #400120), APC anti-human CD47 antibody (Biolegend, Cat #323124), CD47 blocker Ab (from patent WO 2011/143624 A2), Rabit Anti human LILRB2 (Abclonal Cat #A10135), Biotinylated Rabit Anti human SIRPα LsBio Cat (#LS-C370337), Goat anti rabbit IgG (H+L)-HRP conjugate (R&D systems, Cat #170-6515), HRP conjugate-Streptavidin (Pierce Cat #TS21126).

Cell lines—Expi 293F (Gibco, Cat #A14257), HT1080 cell line (ATCC, CCL-121), HT1080-HLA-G, THP1 cell line (ATCC, TIB-202), THP1-EV and THP1-HLA-G. Cells were generated by virus infection with HLA-G expression plasmid or Empty Vector (EV). Briefly, 293T cells were transfected with lentivirus plasmid (containing HLA-G construct or empty vector and reporter reagent GFP). Following 48 hr at 37° C., 5% CO2, the supernatant was collected and used for transduction of HT1080 or THP1 cells. Cells were transduced for 24 hours. HLA-G cells are GFP positive.

Media and Tissue culture reagents—Expi 269 medium (Gibco, Cat #A-14351-01), RPMI 1640 (Biological Industries, Cat #01-100-1A), EMEM (Biological Industries, Cat #01-040-1A), DMEM (Biological Industries, Cat #01-055-1A), DMEM/F-12 (Cat #31330095, Gibco), FCS (Gibco, Cat #12657-029), 10% BSA (Biological Industries, Cat #03-010-1B), EDTA (Sigma, Cat #E7889), TrypLE Express (Gibco, Cat #12604-13), Glutamax (Gibco, Cat #35050-038), Penicillin-Streptomycin (Gibco, Cat #151140-122).

Equipment—FACS Device (Stratadigm, Cytometry S1000EXI).

Manufacturing of recombinant proteins—For comparative functional analysis and production evaluation, several recombinant proteins comprising a wild type (WT) LILRB2 domain (SEQ ID NO: 1) or a mutated LILRB2 domain (referred to herein as “LILRB2 variants”) were produced (see Table 3 hereinbelow). Production was affected in Expi293F cells transfected by pcDNA3.4 expression vectors cloned with the coding sequence for the desired Fc fusion proteins. The sequences were cloned into the vector using restriction enzymes such as EcoRI and HindIII or XbaI and EcoRV, with addition of Kozak sequence and STOP codon plus an artificial signal peptide (MESPAQLLFLLLLWLPDGVHA, SEQ ID NO: 25). The proteins were collected from the supernatant of cell culture and in some cases, proteins were purified by one-step purification using protein A (PA) Poros MabCapture A resin.

TABLE 3
Description of several designed LILRB2 variants

| | First monomer | | Second monomer | |
|---|---|---|---|---|
| | Description | Sequences aa/na | Description | Sequences aa/na |
| DSP216 V5 | LILRB2 -WT- | 37 (1 + 73 + 29)/ | SIRPα- | 35 (27 + 33 + 31)/ |
| | linker-IgG1 hole | 38 (2 + 87 + 30) | linker-IgG1 | 36 (28 + 34 + 32) |
| | Fc | | knob Fc | |
| DSP216 V11 | LILRB2- T50F - | 39 (5 + 73 + 29)/ | | |
| | linker-IgG1 hole | 40 (6 + 87 + 30) | | |
| | Fc | | | |
| DSP216 V12 | LILRB2- V57R - | 41 (7 + 73 + 29)/ | | |
| | linker-IgG1 hole | 42 (8 + 87 + 30) | | |
| | Fc | | | |
| DSP216 V13 | LILRB2- S45Q - | 43 (9 + 73 + 29)/ | | |
| | linker-IgG1 hole | 44 (10 + 87 + 30) | | |
| | Fc | | | |
| DSP216 V14 | LILRB2- I49K - | 45 (11 + 73 + 29)/ | | |
| | linker-IgG1 hole | 46 (12 + 87 + 30) | | |
| | Fc | | | |
| DSP216 V15 | LILRB2- S45Q, | 47 (13 + 73 + 29)/ | | |
| | I49K -linker - | 48 (14 + 87 + 30) | | |
| | IgG1 hole Fc | | | |
| DSP216 V16 | LILRB2- S45Q, | 49 (15 + 73 + 29)/ | | |
| | V57R -IgG1 hole | 50 (16 + 87 + 30) | | |
| | Fc | | | |
| DSP216 V17 | LILRB2- S45R, | 51 (17 + 73 + 29)/ | | |
| | I49F - linker- | 52 (18 + 87 + 30) | | |
| | IgG1 hole Fc | | | |
| DSP216 V18 | LILRB2- S45Y, | 53 (19 + 73 + 29)/ | | |
| | T50K - linker- | 54 (20 + 87 + 30) | | |
| | IgG1 hole Fc | | | |
| DSP216 V19 | LILRB2- S45Y, | 55 (21 + 73 + 29)/ | | |
| | T50N - linker- | 56 (22 + 87 + 30) | | |
| | IgG1 hole Fc | | | |
| DSP216 V20 | LILRB2- S45N, | 57 (23 + 73 + 29)/ | | |
| | T50R - linker- | 58 (24 + 87 + 30) | | |
| | IgG1 hole Fc | | | |
| LILRB2-V5 | LILRB2 -WT- | 37 (1 + 73 + 29)/ | LILRB2 - | 37 (1 + 73 + 29)/ |
| homodimer | linker-IgG1 hole | 38 (2 + 87 + 30) | WT- linker- | 38 (2 + 87 + 30) |
| | Fc | | IgG1 hole Fc | |
| LILRB2-V12 | LILRB2- V57R - | 41 (7 + 73 + 29)/ | LILRB2- | 41 (7 + 73 + 29)/ |
| homodimer | linker-IgG1 hole | 42 (8 + 87 + 30) | V57R - | 42 (8 + 87 + 30) |
| | Fc | | linker-IgG1 | |
| | | | hole Fc | |
| DSP216 WT | LILRB2 -WT- | 101 (1 + 73 + 85)/ | SIRPα - | 94 (27 + 33 + 86)/ |
| | linker-IgG1 NO | 106 (2 + 87 + 92) | linker-IgG1 | 95 (28 + 34 + 93) |
| | LALA hole Fc | | NO LALA | |
| | | | knob Fc | |
| DSP216 V3 | LILRB2 -WT- | 98 (1 + 73 + 85)/ | SIRPα short- | 98 (88 + 33 + 86)/ |
| | linker-IgG1 NO | 99 (2 + 87 + 92) | linker-IgG1 | 99 (89 + 34 + 93) |
| | LALA hole Fc | | NO LALA | |
| | | | knob Fc | |
| DSP216 V6 | LILRB2 -WT- | 37 (1 + 73 + 29)/ | SIRPα short- | 90 (88 + 33 + 31)/ |
| | linker-IgG1 hole | 38 (2 + 87 + 30) | linker-IgG1 | 91 (89 + 34 + 32) |
| | Fc | | knob Fc | |
| DSP216 V12 | LILRB2- V57R - | 41 (7 + 73 + 29)/ | SIRPα short- | 90 (88 + 33 + 31)/ |
| short | linker-IgG1 hole | 42 (8 + 87 + 30) | linker-IgG1 | 91 (89 + 34 + 32) |
| | Fc | | knob Fc | |
| DSP216 V21 | LILRB2 - V57R - | 96 (7 + 73 + 85)/ | SIRPα - | 94 (27 + 33 + 86)/ |
| | linker-IgG1 NO | 97 (8 + 87 + 92) | linker-IgG1 | 95 (28 + 34 + 93) |
| | LALA hole Fc | | NO LALA | |
| | | | knob Fc | |
| DSP216 V21 | LILRB2 - V57R - | 96 (7 + 73 + 85)/ | SIRPα short- | 98 (88 + 33 + 86)/ |
| short | linker-IgG1 NO | 97 (8 + 87 + 92) | linker-IgG1 | 99 (89 + 34 + 93) |
| | LALA hole Fc | | NO LALA | |
| | | | knob Fc | |

SDS-PAGE analysis—Thirty-five μl of cell culture supernatant or 3 μg purified protein from each recombinant protein sample were mixed with loading buffer with or without β-mercaptoethanol (reduced and non-reduced conditions, respectively), heated for 5 minutes at 95° C. and separated on 8% gel electrophoresis SDS-PAGE. Proteins migration on the gel was visualized by e-Stain machinery (GenScript), according to manufacturer instructions.

Western blot analysis—Samples containing the produced heterodimers (50-500 ng per lane) were treated at reducing or non-reducing conditions (in loading buffer with or without β-mercaptoethanol, respectively), heated for 5 minutes at 95° C. and separated on a 8% or 4-20% gradient SDS-PAGE gel. Following, proteins were transferred onto a PVDF membrane and incubated with primary antibodies for one hour or overnight, followed by 1 hour incubation with an HRP-conjugated secondary antibody. Signals were detected following ECL development.

Flow cytometry—to evaluate binding of the recombinant proteins to HLA-G expressed on cell's surface, HT1080-WT cells or THP-1-EV cells, or HT1080 or THP-1 cells overexpressing human HLA-G were incubated with serial dilutions of the produced recombinant protein for 30 minutes at 4° C., followed by immuno-staining with fluorescently labeled antibody specific to the IgG1 backbone and flow cytometry analysis. To test binding specificity, cells underwent pre-incubation with a blocker antibody against human HLA-G at concentration of 5 μg/ml, for 1 hour at 37° C. prior to incubation with the recombinant protein. MFI values were used to create a binding curve graph with a GraphPad Prism software).

### Results

Several heterodimer proteins comprising either a WT LILRB2 (SEQ ID NO: 1) or a mutated LILRB2 (referred to herein as “LILRB2 variant”)-Fc fusion and a SIRPα-Fc fusion were produced and analyzed. The heterodimer comprising WT LILRB2 is referred to herein as “DSP216-V5”, while the heterodimers comprising LILRB2 variants are referred to herein as “DSP216-V11”, “DSP216-V12”, “DSP216-V13”, “DSP216-V14”, “DSP216-V15”, “DSP216-V16”, “DSP216-V17”, “DSP216-V18”, “DSP216-V19”, “DSP216-V20”. In addition, two LILRB2 homodimers proteins were produced and analyzed: a homodimer comprising WT LILRB2, referred to herein as “LILRB-V5-Fc” and a homodimer comprising a LILRB2 variant, referred to herein as “LILRB2-V12-Fc. Full description of each heterodimer/homodimer is provided in Table 3 hereinabove).

As demonstrated in FIG. 2A, a high proportion of the protein of the expected heterodimer molecular weight form was observed under non-reducing conditions and the expression of the two subunits was confirmed in reducing conditions.

Following, binding of the produced heterodimers to HLA-G was determined by flow-cytometry analysis using HT1080-WT and HT1080 overexpressing HLA-G (HT1080-HLA-G) and THP1 mock-transduced with an empty vector (THP1-EV) or overexpressing HLA-G-(THP1-HLA-G) cell lines and anti-hIgG1 as a detection antibody (FIGS. 3A-F). A significantly higher binding was observed to HLA-G overexpressing cells compared to the WT or EV cells expressing hCD47-only (FIGS. 3A-B). Furthermore, the binding to HLA-G expressing cells of heterodimers comprising a LILRB2 variant domain was significantly higher than the binding of the heterodimer comprising a WT LILRB2 domain (DSP216-V5) (FIG. 3C).

As HT1080-HLA-G cells express both HLA-G and CD47, compared to the HT1080 cells that express only CD47 (FIG. 3A), the specific binding of the produced heterodimers to HLA-G was further tested using an anti-HLA-G blocking antibody. The anti-HLA-G binding-inhibition levels on the heterodimers comprising LILRB2 variants were higher compared to the inhibition on the heterodimer comprising WT LILRB2, indicating their superiority in specific binding to HLA-G (FIGS. 3D-F).

Binding of LILRB2-V5 or LILRB2-V12 homodimers to HLA-G was determined by flow-cytometry analysis using HT1080-WT and HT1080 overexpressing HLA-G (HT1080-HLA-G), and anti-hIgG1 as a detection antibody (FIG. 4). A significantly higher binding of LILRB2-V12 homodimer was observed to HLA-G overexpressing cells compared to WT cells expressing hCD47-only (FIG. 4). Furthermore, the binding of homodimers comprising a LILRB2-V12 variant domain to HLA-G expressing cells was significantly higher than the binding of the homodimer comprising a WT LILRB2 domains (LILRB2-V5, FIG. 4). Moreover, the anti-HLA-G binding-inhibition levels on the homodimers comprising a LILRB2-V12 variant was higher compared to the inhibition on the homodimer comprising a WT LILRB2, indicating their superiority in specific binding to HLA-G (FIG. 4).

Additional heterodimer proteins comprising a fusion of a LILRB2 variant fused to an Fc domain with or without LALA mutations (SEQ ID 29 and 85, respectively) and a fusion of SIRPα (SEQ ID 27 or 88 to an Fc with or without LALA mutations (SEQ ID 31 and 86, respectively) were produced and analyzed. Full description of each heterodimer is provided in Table 3 hereinabove.

As demonstrated in FIG. 9, a high proportion of the protein of the expected heterodimer molecular weight form was observed under non-reducing conditions and the expression of the two subunits was confirmed in reducing conditions. Further, as demonstrated in FIG. 10, western blot analysis confirmed that the produced DSP216-V12, DSP216-V12 short, DSP216-V21 and DSP216-V21 short contained domains of SIRPα and LILRB2.

Binding of DSP216-WT, DSP216-V3, DSP216-V6, DSP216-V12 short, DSP216-V21 and DSP216-V21 short heterodimers to HLA-G was determined by flow-cytometry analysis using HT1080-WT and HT1080 overexpressing HLA-G (HT1080-HLA-G), and anti-hIgG1 as a detection antibody. As shown in FIG. 11 a significantly higher binding of all tested DSP216 heterodimers was observed to HLA-G overexpressing cells compared to WT cells expressing hCD47-only. Furthermore, the binding of a DSP216 variant containing the V57R substitution in the LILRB2 sequence (DSP216-V21, DSP216-V21 short and DSP216-V12 short) to HLA-G expressing cells was significantly higher compared to binding of the heterodimer comprising a WT LILRB2 domain (DSP216-WT, DSP216-V3 and DSP216-V6). Moreover, a significant binding-inhibition was demonstrated by an anti-HLA-G blocker antibody, indicating the specific binding to HLA-G.

### Example 3

### Binding of the LILRB2 Variants to Human HLA-G

To further verify superiority of the LILRB2 variants, the binding affinity of the LILRB2 variants to human HLA-G and competition with the WT LILRB2 is determined by Surface Plasmon Resonance (SPR) assays.

### Example 4

### LILRB2 Variants Block Native LILRB2-HLA-G Binding

Endogenous LILRB2 is expressed on human monocytes, B cells and at lower levels on the cell surface of myeloid and plasmacytoid dendritic cells (Katz H R. Adv Immunol. (2006) 91:251-272; Kang X, et al. Cell Cycle. (2016) 15 (1): 25-40).

Endogenous LILRB1 is expressed on human macrophages, some T cells, NK cells, B cells, monocytes, various dendritic cell subsets including myeloid, plasmacytoid and tolerogenic DCs (Katz H R. Adv Immunol. (2006) 91:251-272; Kang X. et al. Cell Cycle. (2016) 15 (1): 25-40).

Endogenous HLA-G is predominantly expressed on cytotrophoblasts in the placenta; however, it has been shown that cells associated with several pathologic conditions (e.g. cancer, viral infections, auto-immune and inflammatory diseases, GvHD) express HLA-G (Contini P, et al., Front Immunol. (2020) 11:1613, PMID: 32983083; Morandi F, et al., J Immunol Res. (2016) 2016:4326495, PMID: 27652273).

Interaction of LILRB2, as well as LILRB1, expressed on immune cells with HLA-G, their native ligand, initiates a signal-transduction pathway which inhibits activity of the immune cells. The LILRB2 comprising proteins are designed to block the interaction of endogenous LILRB2, as well as LILRB1, expressed on immune cells with HLA-G expressed on e.g. tumor cells.

The effectiveness of the produced proteins comprising LILRB2 variants as blockers of these interactions is evaluated by an ELISA assay and compared to WT LILRB2. To this end, ELISA plates are coated with a recombinant human HLA-G. Following, plates are washed and incubated for 1 hour with different concentrations of the produced LILRB2-comprising proteins or the positive control anti-HLA-G blocker antibody. LILRB2-Fc (mIgG), or LILRB1-Fc (mIgG) is added followed by additional incubation, and the plate is then washed and blotted with anti-mouse IgG-HRP and TMB substrate according to a standard ELISA protocol. Plates are analyzed using a plate reader (Thermo Scientific, Multiscan FC) at 450 nm, with reference at 620 nm.

### Example 5

### Ligand Binding ELISA

The binding of LILRB2 to its counterparts, HLA-G, is tested by a binding ELISA assay.

hHLA-G is bound on the surface of a plastic plate, LILRB2 is added and allowed to bind to the immobilized hHLA-G. Following washing, HRP anti hIgG1 is added and allowed to bind to the Fc backbone of the molecule. Detection is affected with a TMB substrate according to standard ELISA protocol using a Plate reader (Thermo Scientific, Multiscan FC) at 450 nm, with reference at 540 nm.

### Example 6

### In-Vivo Anti-Tumor Effect of the LILRB2 Variants

Three different in-vivo mouse models are used for testing the efficacy of the produced heterodimers comprising LILRB2 variants in treating cancer:
1. NSG mice inoculated with human stem cells or with human PBMCs or with immobilized human PBMCs and with human tumor cells expressing CD47 and HLA-G.
2. Nude-SCID mice inoculated with human tumor cells expressing CD47 and HLA-G.
3. Syngeneic mouse tumor models using mice inoculated with mouse cancer cell line expressing CD47 and HLA-G and the surrogate mouse protein of the tested heterodimer.

In all models, mice are inoculated with tumor cells intravenously (IV), intraperitoneally (IP), subcutaneously (SC) or orthotopically. Once the tumor is palpable (˜80 mm³), mice are treated IV, IP, SC or orthotopically, with different doses and different regimens of the produced heterodimer.

Mice are followed for weights and clinical signs. Tumors are measured few times a week by a caliper; and tumor volume is calculated according to the following equation: V=length X width²/2. Mice Weight is measured routinely. Tumor growth and survival are monitored through the whole experiment.

Infiltration and sub-typing of immune cells in the tumor is tested by resecting the tumor or draining lymph nodes, digestion and immune phenotyping using specific antibodies staining and flow cytometry analysis. Additionally, or alternatively, infiltration of immune cells or necrotic grade of tumors is determined by resecting the tumors, paraffin embedding and sectioning for immunohistochemistry staining with specific antibodies.

At sacrificing, mice organs are harvested and embedded into paraffin blocks for H&E and IHC staining.

Blood samples are taken from mice at different time points, according to common procedures, for the following tests: PK analysis, cytokines measurements in plasma, FACS profiling of blood cells sub-populations in circulation, hematology testing, serum chemistry testing, anti-drug-antibody (ADA) analysis and neutralizing antibodies analysis (NAB).

### Example 7

### The Effect of the LILRB2 Variants on M-CSF Dependent Macrophage Maturation

The LILRB2 domain of the produced proteins is designed to block the immunosuppressive signals induced by HLA-G expressed on tumor or immune cells towards the endogenous LILRB1 and LILRB2 expressed on antigen presenting cells (APCs) such as macrophages and dendritic cells, by competing and blocking their interaction. M1 macrophages show anti-tumor activity, while M2 macrophages have been reported to promote tumor progression. M-CSF is known to drive differentiation of monocytes to naïve M0 (M2 like) macrophages that can be subsequently polarized to pro-inflammatory (M1 macrophages) or anti-inflammatory (M2 macrophages) phenotypes by the different activating stimuli (Chistiakov D A, et al., J. Cell. Mol. Med. (2015) 19 (6): 1163-1173 PMID: 25973901). Blocking of LILRB2 with an antagonistic antibody during M-CSF dependent macrophage maturation was shown to lead to a rounder and tightly adherent M1 (anti-tumor) phenotype with lower expression of CD14 and CD163 (Chen H M, et al., J Clin Invest. (2018) 128 (12): 5647-5662. PMID: 30352428). After stimulation of the generated macrophages with LPS, enhanced secretion of the pro-inflammatory cytokine TNFα and reduced secretion of anti-inflammatory IL-10 was detected (Chen H M et al.).

The effect of the produced recombinant proteins comprising LILRB2 variants on M-CSF dependent macrophage maturation was evaluated using a flow cytometry-based detection of M1/M2 markers and by measurement of TNFα and IL-6 (known M1 related cytokines) release after stimulation of LPS pre-treated macrophages.

### Materials

Reagents—DSP216-V12, DSP216-V12 Short, fresh buffy coat samples from healthy donors (Hadassah Bank blood), Ficoll-Paque™ PLUS 1.077 (Cytiva Cat #GE-17-1440-03), human trustain FcX (Biolegend, Cat #422302), PBS (Sartorius, Cat #20-023-1A), EDTA (Sigma, Cat #E7889), sodiume azide (Sigma, Cat #S2002), 10% BSA (Sartorius, Cat #03-010-1B or Biological Industries, Cat #03-010-1B), BD Cytometric Bead Array (CBA) (BD, Cat #551809), RPMI 1640 (Biological Industries, Cat #01-100-1A), DMEM (Biological Industries, Cat #01-055-1A), FBS (Gibco, Cat #12657-029), TrypLE Express (Gibco, Cat #12604-13), glutamax (Gibco, Cat #35050-038), penicillin-Streptomycin (Gibco, Cat #151140-122), human recombinant M-CSF (R&D, Cat #216-MC-100).

Antibodies—Anti HLA-G Tizona-like (described in US Patent Application Publication No. 20200102390), BV785 anti-human CD11b (Biolegend, Cat #301346), APC anti-human CD163 (Biolegend, Cat #333610), APC anti-human CD206 (Biolegend, Cat #321110), APC anti-human HLA-DR (Biolegend, Cat #307610), APC mouse IgG1 (Biolegend, Cat #40120) Iso-C for CD163 and CD206, APC mouse IgG2a (Biolegend, Cat #400220) Iso-C for HLA-DR.

Human Monocytes—PBMCs were purified from two fresh buffy coat samples by Ficoll gradient according to the manufacturer instructions. Cells were seeded in T75 flasks (1×10⁸ cells per flask) with RPMI medium and incubated at 37° C. 5% CO2. Following 2 hours of incubation, medium was replaced with fresh RPMI supplemented with 50 ng/ml M-CSF. Following 6 days of incubation the cells are defined as M0 macrophages (Tarique A A, et al., American J of Respiratory Cell and Molecular Biology (2015) 53 (5): 676-688, PMID: 25870903).

Cell lines—HT1080 cells overexpressing HLA-G (HT1080-HLA-G), as described in Example 2 hereinabove. Of note, the HT1080 overexpressing HLA-G cells are GFP positive.

### Methods

The effect of DSP216-V12 on M0 macrophage polarization was evaluated by co-culturing the macrophages with HT1080 HLA-G cells. Following 6 days incubation with M-CSF, M0 macrophages were seeded in 6 well plates (250,000 cells per well) with RPMI and 50 ng/ml M-CSF for overnight incubation. The next day, HT1080-HLA-G cells were incubated for 1 hour at 37° C. with 2, 4 or 10 μg/ml DSP216-V12 or 1.5 μg/mL anti-human HLA-G as a positive control (equal molarity to 2 μg/ml DSP216-V12) and then were seeded on top of the M0 macrophages at a 2:1 E:T ratio (250,000 M0 macrophages: 125,000 HT1080-HLA-G cells). Following 24 hours incubation, the cells were collected, and the expression of established polarization markers was determined by flow cytometry (CytoFlex B53000 CytoFLEX B5-R3-V5). Specifically, macrophages were gated as CD11b positive cells and the surface expression of the M2 markers CD163 and CD206, and the M1 marker HLA-DR, were evaluated. In addition, supernatants were collected for determining cytokines secretion. MFI values were used to analyze FACS data (FlowJo v10.8.1 software) and FCAP Array v3.0 software was used to analyze CBA data of cytokines levels in the supernatants.

### Results

Following treatment with DSP216-V12 or DSP216-V12 short (heterodimer proteins comprising a LILRB2 variant-Fc fusion and a SIRPα-Fc fusion, see Table 3 for full description) the expression of CD163 and CD206 M2 associated markers in M-CSF polarized monocytes co-cultured with HT1080 HLA-G cells, was downregulated in a dose-depended manner, as compared to the untreated or the anti-HLA-G control groups (FIGS. 5A-B and 12A-B), while the expression of the M1 associated marker, HLA-DR, was upregulated (FIGS. 5C and 12C). Further, treatment with DSP216-V12 induced secretion of the M1 associated cytokines, TNFα and IL-6 (FIGS. 6A-B).

Taken together, the results indicated that DSP216-V12 and DSP216-V12 short are capable of converting M0 (M2-like) macrophages to M1 macrophages.

### Example 8

### The Effect of LILRB2 Variants on Macrophages and Polymorphonuclear Cells

The LILRB2 domain of the proteins is designed to block the immunosuppressive signals induced by HLA-G expressed on tumor or immune cells towards the endogenous LILRB1 and LILRB2 expressed on APCs such as macrophages and dendritic cells, by competing and blocking their interaction. This blockage of the HLA-G “don't eat me signal” induces tumor cell phagocytosis and prevents the inhibitory HLA-G-LILRB1/2 signaling between cancer and immune cells, in turn enhancing phagocytosis.

The effect of the produced recombinant proteins comprising LILRB2 variants on tumor cell phagocytosis may be evaluated by mixing macrophages with CFSE or Cell trace violet (CTV)-labelled cancer cells that were pre-incubated in the presence of the produced recombinant proteins at different concentrations. Following various incubation times (e.g. 3 hours), macrophages are stained with an anti-CD11b antibody and phagocytic uptake of the stained cancer cells is evaluated by microscopy or flow cytometry analysis.

### Materials

Reagents—DSP216-V12, fresh buffy coat samples from healthy donors (Sanquin Bank blood), Ficoll Lymphoprep (Serumwerk Bernburg AG for Alere Technologies AS, Oslo, Norway, 04-03-9391/02), TripLE Express (gibco, cat #12604021), FC receptor blocking solution (Nanogen), PBS (in house, umcg pharmacy), EDTA (sigma Aldrich, cat #E9884-1 KG), sodium azide (BDH Chemical LTD), BSA (BSA Fraction V, Roche, cat #10735094001), RPMI 1640 (gibco, 52400-025), FBS (gibco), human recombinant M-CSF (immunotools, cat #11343115), human recombinant IL-10 (immunotools, cat #1134107), CellTrace™ Violet Cell Proliferation Kit (Thermo Fisher Scientific, Cat #C34557).

Antibodies—Anti HLA-G Tizona-like (described in US Patent Application Publication No. 20200102390). APC anti-human CD47 (Biolegend, Cat #323124) APC anti-human CD11b (Biolegend, Cat #301310), APC anti-human CD163 (e-Bioscience, Cat #17-1639-41), APC anti-human LILRB2 (Biolegend, Cat #338708), APC mouse IgG4 (Biolegend, Cat #403706) Iso-C for HLA-G, APC mouse IgG1 (Biolegend, Cat #40120) Iso-C for CD47, CD11b and CD163, APC rat IgG2a (Biolegend, Cat #400512) Iso-C for LILRB2, CD47 blocking Ab Inhibrix-like (described in US Patent Application Publication No. 2015/0183874 A1).

Human Monocytes-Samples were obtained from fresh buffy coat samples by purifying on Ficoll gradient according to the manufacturer instructions. Cells were seeded in 6 well plates (5×10⁶ cells per well in 2 mL) with RPMI medium supplemented with 10% FCS and 50 ng/mL M-CSF and incubated at 37° C., 5% CO2. Following overnight incubation, medium was replaced with fresh RPMI supplemented with 10% FCS and 50 ng/ml M-CSF. After 6 days of incubation, medium was replaced with RPMI supplemented with 10% FCS and 50 ng/ml IL-10. Following 48 hours incubation, the cells were defined as M2c macrophages.

Cell lines—721.221 cells (Shimizu, Y., and R. DeMars, J. Immunol. (1989) 142:3320; Markel G. et al., J Immunol (2002) 168 (6): 2803-2810) overexpressing HLA-G (721.221-HLA-G). Cells were generated by virus infection with HLA-G and GFP or GFP only (Empty Vector-EV) expression plasmids.

### Methods

The effect of DSP216-V12 on M2c macrophage phagocytosis was evaluated by co-culturing the macrophages with 721.221-HLA-G or 721.221-EV cells. 721.221-HLA-G cells and 721.221-EV cells were stained with CTV and 250,000 cells were seeded in FACS tubes and incubated with medium containing 2.5, 5 or 10 μg/mL DSP216-V12 or 6 μg/mL CD47 blocking antibody for 20 minutes at 4° C. Fifty thousand M2c macrophages were added to each tube containing cancer cells. Following 3 hours of incubation, the macrophages were stained with an APC-CD11b antibody, and the percentage of phagocytosis was determined as the fraction of CD11b+ cells that were positive for CTV. In parallel, CD47 and HLA-G expression was confirmed by testing their expression on the surface of 721.221-HLA-G and -EV cells, using FACS; and CD11b, CD163 and LILRB2 expression levels were tested on M2c macrophages. FACS data was analyzed by FlowJo v10.8.1 software and statistical analysis was done with GraphPad Software (Prism 9 for macOS, Version 9.4.1).

### Results

Following treatment with DSP216-V12 (a heterodimer protein comprising a LILRB2 variant-Fc fusion and a SIRPα-Fc fusion) the phagocytosis of 721.221 EV and 721.221-HLA-G cells by M2c macrophages increased in a dose-dependent manner. For the CD47⁺ HLA-G-721.221-EV cells, the increase in phagocytosis was not significant at any DSP216-V12 concentration, whereas CD47 blocking antibody increased the phagocytosis significantly (**p=0.0028) (FIG. 13A). For the CD47⁺ HLA-G⁺721.221-HLA-G cells, the increase in phagocytosis was significant with 10 μg/mL DSP216-V12 (*p=0.0465), whereas CD47 blocking antibody did not increase the phagocytosis significantly (p=0.1654) (FIG. 13B).

Taken together, the results indicated that DSP216-V12 is capable of significantly increasing phagocytosis of CD47+/HLA-G⁺ cells by M2c macrophages.

This effect arises from blocking the CD47/SIRPα axis together with the HLA-G/LILRB1/2 axis, as blocking only the CD47/SIRPα axis with CD47 blocking antibody has a lower effect on phagocytosis of CD47+/HLA-G⁺721.221-HLA-G.

### Example 9

### NK Cells Cytotoxic Activation by the LILRB2 Variants

Natural killer (NK) cells induce direct cytotoxicity or secretion of cytokine/chemokine without recognizing a specific antigen as B and T cells. NK cytotoxicity plays an important role in immune response against infected cells, malignancy and stressed cells, and is involved in the pathologic process of various diseases.

Numerous assays known in the art are used to determine the effect of the produced recombinant proteins comprising LILRB2 variants on NK activation, including, but not limited to:

Cytotoxicity assay-Killing of Target cells by NK cells (effector cells) in a co-culture assay. % of killing is analyzed by flow cytometry analysis (FACS). Target cells are placed in 96-wells plates and incubated with pre-labeled primary NK cells at various effector-target (E:T) ratios in the presence of the produced recombinant proteins at different concentrations. NK cells are cultured with 1000 U/mL IL-2 for 48 hours before the assay. Cells are harvested following 4 and 24 hours and assayed by flow cytometry. The numbers of target cells recovered from cultures without NK cells are used as a reference.

Cytotoxicity assay-Killing of Target cells by NK cells (effector cells) in a co-culture assay in the presence of the produced recombinant proteins at different concentrations. % of killing is determined by an Incucyte machine using labeled target cells and caspase sensitive florescent substrate.

Secretion of inflammatory cytokines-primary NK cells are stimulated with various target cells at various ratio for 24 hours in the presence of the produced recombinant proteins at different concentrations. The levels of interferon γ (IFN-γ) and granulocyte-macrophage colony-stimulating factor (GM-CSF) in cell-free culture supernatants are determined with ELISA or Cytometric Bead Array (CBA).

### Example 10

### Specific Binding of Heterodimers Comprising LILRB2 Variants and SIRPα to Cells Expressing Both HLA-G and CD47

### Materials and Methods

Reagents—Alexa Fluor 647 labelled-DSP216-V12, fresh buffy coat samples from four healthy donors (Hadassah Bank blood), fresh whole blood from healthy donors (Hadassah Bank blood), Ficoll-Paque™ PLUS 1.077 (Cytiva Cat #GE-17-1440-03), human trustain FcX (Biolegend, Cat #422302), RBC Lysis Buffer (Invitrogen, Cat #00-4333-57), PBS (Sartorius, Cat #20-023-1A), EDTA (Sigma, Cat #E7889), sodiume azide (Sigma, Cat #S2002), 10% BSA (Sartorius, Cat #03-010-1B or Biological Industries, Cat #03-010-1B), Alexa Flour 647 Microscale Protein Labelling kit (Invitrogen, Cat #A30009), RPMI 1640 (Biological Industries, Cat #01-100-1A), DMEM (Biological Industries, Cat #01-055-1A), FCS (Gibco, Cat #12657-029), TrypLE Express (Gibco, Cat #12604-13), glutamax (Gibco, Cat #35050-038), penicillin-Streptomycin (Gibco, Cat #151140-122).

Antibodies—BV421 anti-human CD45 (Biolegend, Cat #304032), APC Mouse IgG1 k (Biolegend, Cat #400120), APC anti-human CD47 antibody (Biolegend, Cat #323124).

Human PBMCs and RBCs—PBMCs were purified from four fresh buffy coat samples by Ficoll gradient according to the manufacturer's instructions. Whole blood from four healthy donors was diluted 1:500 in PBS and considered as RBCs.

Cell lines—As described in Example 2 hereinabove. Note, the HT1080 cells overexpressing HLA-G are GFP positive.

CD47 expression—RBCs, PBMCs or HT1080 HLA-G cells were incubated with an anti-human CD47 antibody and analyzed by flow cytometry (CytoFlex B53000 CytoFLEX B5-R3-V5). MFI values were used to create a representative graph with a GraphPad Prism software. Binding analysis-_100 μL/well of whole blood sample diluted 1:500 in PBS (considered as a RBCs sample containing about 1×10⁶ RBCs) were mixed with 25,000-50,000/well of purified PBMCs and 25,0000-50,000 HT1080 cells overexpressing human HLA-G cells (HT1080-HLA-G). Mixed cells were incubated for 30 minutes at 4° C. with serial dilutions of Alexa Fluor 647 labelled-DSP216-V12. Following incubation, cells were washed and were immuno-stained with a BV421 anti-human CD45 antibody and analyzed by flow cytometry (CytoFlex B53000 CytoFLEX B5-R3-V5). MFI values were used to create a binding curve graph with a GraphPad Prism software).

### Results

CD47 was highly expressed on the surface of HT1080-HLA-G cells (FIG. 7). Low expression levels of CD47 were also detected on the surface of PBMCs and RBCs from healthy donors (FIG. 7). Binding of Alexa Fluor 647 labelled-DSP216-V12 was determined by flow-cytometry using a mix of RBC, PBMCs and HT1080-HLA-G cells. An anti-hCD45 antibody was used to gate RBCs (CD45 negative cells) and PBMCs (CD45 positive cells) and GFP was used to gate HT1080-HLA-G cells. While DSP216-V12 bound HT1080-HLA-G cells in a dose dependent manner, negligible binding was demonstrated to PBMCs, and no binding was observed to RBCs (FIGS. 8A-B).

### Example 11

### Design of LILRB1 Variants

### Materials and Methods

A homology analysis comparing human LILRB2 (UniProt Number Q8N423) and LILRB1 (UniProt Number Q8NHL6) was effected using the alignment algorithm Blast.

Following, a structural analysis of WT LILRB1 (SEQ ID NO: 102) was affected in order to identify amino acid substitutions that may optimize and increase its stability and binding affinity to HLA-G (SEQ ID NO: 3), which included the following steps:
1. Preparation of a complex structure of both LILRB1 and HLAG using PDB ID: 2DYP, 3D2U complex structures.
2. Structural analysis to identify potential interacting points between LILRB1 and HLA-G (FIG. 14) PDB IDs: 2DYP (HLA-G) and 3D2U (LILRB1) complex structures.
3. Identifying amino acids in LILRB1 interface which interact with Phe195 of the HLA-G [was identified as specific for HLA-G, i.e. not present in HLA-A, HLA-B or HLA-C (FIG. 1D)].
4. In-silico evaluating the effect of mutating V55 of LILRB1 sequence set forth in SEQ ID NO: 102.

### Results

A high sequence homology was identified between human LILRB2 (UniProt Number Q8N423) and LILRB1 (UniProt Number Q8NHL6), especially in the D1 (Ig-like C2-type 1) domain (SEQ ID NOx: 104 and 105, respectively) (FIG. 14). Interestingly, a very high homology was detected in the region containing the amino acid residues identified in Example 1 hereinabove as defining the interface between LILRB2 and the HLA-G Phe195 interacting residue (residues S45, I49, T50 and V57 of SEQ ID NO: 1). This prompted the present inventors to evaluate whether mutating the corresponding amino acids of LILRB1 (namely, T43, 147, T50 and V55 of SEQ ID NO: 102) will also have a significant positive influence i.e., lower ΔG, on binding energy.

To this end, a structural analysis of HLA-G (SEQ ID NO: 3)-LILRB1 (SEQ ID NO: 102) interacting interface and specifically with Phe195 in HLA-G was performed (FIGS. 15A-B). Following, in-silico analysis indicated that substituting residue V55 of the LILRB1 (SEQ ID NO: 102) to e.g. arginine (R), is likely to stabilize the LILRB1/HLA-G interaction (FIGS. 15C-D).

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

It is the intent of the applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

1. A LILRB polypeptide capable of binding an HLA-G polypeptide as set forth in SEQ ID NO: 3 and having at least one mutation located within amino acid positions 40-60 of a D1 domain of LILRB, wherein said LILRB polypeptide has an increased stability and/or increased affinity to said HLA-G compared to a LILRB polypeptide of the same length and sequence not comprising said at least one mutation.

2. (canceled)

3. The LILRB polypeptide of claim 1, wherein said LILRB is LILRB2 and said at least one mutation is at an amino acid position selected from the group consisting of S45, I49, T50 and V57 corresponding to SEQ ID NO: 1.

4. (canceled)

5. The LILRB2 polypeptide of claim 3, wherein said mutation in S45 comprises a S45R, S45N, S45Q, S45H, S45L, S45K, S45M, S45F, S45W or S45Y mutation, said mutation in I49 comprises a I49R, I49K, I49F or I49Y mutation, said mutation in T50 comprises a T50R, T50N, T50L, T50K, T50F, T50W or T50Y mutation, and/or said mutation in V57 comprises a V57R, V57K, V57F or V57W mutation.

6. The LILRB2 polypeptide of claim 3, wherein said mutation in S45 comprises a S45Q mutation, said mutation in I49 comprises a I49K mutation, said mutation in T50 comprises a T50F mutation, and/or said mutation in V57 comprises a V57R mutation.

7. The LILRB2 polypeptide of claim 1, wherein said at least one mutation comprises at least two mutations.

8. The LILRB2 polypeptide of claim 3, wherein said at least one mutation comprises a mutation at said S45 and an additional mutation at said I49, T50 and/or V57.

9. The LILRB2 polypeptide of claim 8, comprising S45N and T50R mutations, S45Y and T50K mutations, S45R and I49F mutations, S45Q and V57R mutations, S45Q and I49K mutations, or S45Y and T50N mutations.

10-11. (canceled)

12. The LILRB2 polypeptide of claim 3, wherein said LILRB2 polypeptide amino acid sequence is as set forth in SEQ ID NO: 5, 7, 9, 11, 13, 15, 17, 19, 21 or 23.

13. The LILRB polypeptide of claim 1, wherein said LILRB is LILRB1 and said at least one mutation is at an amino acid position selected from the group consisting of T43, 147, T48 and V55 corresponding to SEQ ID NO: 102.

14. The LILRB1 polypeptide of claim 13, wherein said mutation in T43 comprises a T43R, T43N, T43Q, T43H, T43L, T43K, T43M, T43F, T43W or T43Y mutation, said mutation in I47 comprises a I47R, 147K, 147F or 147Y mutation, said mutation in T48 comprises a T48R, T48N, T48L, T48K, T48F, T48W or T48Y mutation, and/or said mutation in V55 comprises a V55R, V55K, V55F or V55W mutation.

15. The LILRB1 polypeptide of claim 13, wherein said mutation in V55 comprises a V55R mutation.

16-17. (canceled)

18. A composition of matter comprising the LILRB polypeptide of claim 1 and a non-proteinaceous moiety attached to said LILRB polypeptide.

19-20. (canceled)

21. A fusion polypeptide comprising the LILRB polypeptide of claim 1 attached to a heterologous proteinaceous moiety.

22-25. (canceled)

26. A dimer comprising the LILRB polypeptide of claim 1.

27. (canceled)

28. The dimer of claim 26, wherein a first monomer of said heterodimer comprises said LILRB polypeptide and a second monomer comprising an amino acid sequence of a protein selected from the group consisting of SIRPα, PD1, TIGIT and SIGLEC10, wherein said amino acid sequence is capable of binding its natural binding pair.

29-32. (canceled)

33. A polynucleotide encoding the LILRB polypeptide of claim 1.

34. (canceled)

35. A host cell comprising the LILRB polypeptide of claim 1.

36. A method of producing a polypeptide, the method comprising introducing the polynucleotide of claim 33 to a host cell.

37. (canceled)

38. A method of treating a disease associated with pathologic cells expressing HLA-G in a subject in need thereof, the method comprising administering to the subject a therapeutically effective amount of the LILRB polypeptide of claim 1, thereby treating the disease in the subject.

39-41. (canceled)

42. A method of activating immune cells, the method comprising in-vitro activating immune cells in the presence of the LILRB polypeptide of claim 1.

43. (canceled)


SEQUENCE LISTING (106 sequences)

SEQ ID NO: 1 - protein, 398 aa, synthetic construct
  REGION 1..398: note = WT LILRB2 (AA 22-419) AA
QTGTIPKPTLWAEPDSVITQGSPVTLSCQGSLEAQEYRLYREKKSASWITRIRPELVKNGQFHIPSITWEHTGRYGCQYYSRARWSELSDPLVLVMTGAYPKPTLSAQPSPVVTSGGRVTLQCESQVAFGGFILCKEGEEEHPQCLNSQPHARGSSRAIFSVGPVSPNRRWSHRCYGYDLNSPYVWSSPSDLLELLVPGVSKKPSLSVQPGPVVAPGESLTLQCVSDVGYDRFVLYKEGERDLRQLPGRQPQAGLSQANFTLGPVSRSYGGQYRCYGAHNLSSECSAPSDPLDILITGQIRGTPFISVQPGPTVASGENVTLLCQSWRQFHTFLLTKAGAADAPLRLRSIHEYPKYQAEFPMSPVTSAHAGTYRCYGSLNSDPYLLSHPSEPLELVVS

SEQ ID NO: 2 - DNA, 1194 bp, synthetic construct
  misc_feature 1..1194: note = NA OF WT LILRB2
  source 1..1194: mol_type = other DNA
cagaccggcacaatcccaaagcccaccctgtgggccgagcctgattccgtgatcacccagggctctccagtgacactgtcctgccagggctctctggaggcccaggagtaccggctgtatagagagaagaagtctgccagctggatcacccggatcagacctgagctggtgaagaacggccagtttcacatcccaagcatcacctgggagcacacaggccggtacggatgccagtactattcccgggccagatggagcgagctgtccgaccctctggtgctggtcatgaccggcgcctatcctaagccaacactgagcgcccagccatcccctgtggtgaccagcggcggcagagtgacactgcagtgtgagtcccaggtggccttcggcggctttatcctgtgcaaggagggcgaggaggagcacccacagtgtctgaacagccagccacacgcccggggcagctccagagccatcttctccgtgggacccgtgagcccaaaccggagatggagccaccggtgctacggctatgacctgaatagcccttacgtgtggtctagcccatccgatctgctggagctgctggtgcccggcgtgtccaagaagccttccctgtctgtgcagccaggaccagtggtggcaccaggagagtctctgaccctgcagtgcgtgagcgacgtgggctacgatcggttcgtgctgtataaggagggagagagggatctgaggcagctgccaggcagacagccacaggccggcctgagccaggccaactttacactgggcccagtgagcaggtcctatggcggacagtacaggtgctatggagcacacaatctgtcctctgagtgttctgcccccagcgaccccctggacatcctgatcaccggccagatcaggggcacacccttcatctccgtgcagcctggaccaaccgtggcctctggcgagaacgtgacactgctgtgccagtcttggcgccagttccacacctttctgctgacaaaggcaggagcagcagacgcaccactgaggctgcgcagcatccacgagtaccccaagtatcaggccgagtttccaatgtctccagtgaccagcgcccacgcaggcacatacaggtgttatggcagcctgaacagcgacccctacctgctgagccacccttccgagccactggagctggtggtgagc

SEQ ID NO: 3 - protein, 338 aa, synthetic construct
  REGION 1..338: note = HLA-G AA
MVVMAPRTLFLLLSGALTLTETWAGSHSMRYFSAAVSRPGRGEPRFIAMGYVDDTQFVRFDSDSACPRMEPRAPWVEQEGPEYWEEETRNTKAHAQTDRMNLQTLRGYYNQSEASSHTLQWMIGCDLGSDGRLLRGYEQYAYDGKDYLALNEDLRSWTAADTAAQISKRKCEAANVAEQRRAYLEGTCVEWLHRYLENGKEMLQRADPPKTHVTHHPVFDYEATLRCWALGFYPAEIILTWQRDGEDQTQDVELVETRPAGDGTFQKWAAVVVPSGEEQRYTCHVQHEGLPEPLMLRWKQSSLPTIPIMGIVAGLVVLAAVVTGAAVAAVLWRKKSSD

SEQ ID NO: 4 - protein, 119 aa, synthetic construct
  REGION 1..119: note = Beta2 microglobulin
MSRSVALAVLALLSLSGLEAIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDM

SEQ ID NO: 5 - protein, 398 aa, synthetic construct
  REGION 1..398: note = Variant V11 T71F
QTGTIPKPTLWAEPDSVITQGSPVTLSCQGSLEAQEYRLYREKKSASWIFRIRPELVKNGQFHIPSITWEHTGRYGCQYYSRARWSELSDPLVLVMTGAYPKPTLSAQPSPVVTSGGRVTLQCESQVAFGGFILCKEGEEEHPQCLNSQPHARGSSRAIFSVGPVSPNRRWSHRCYGYDLNSPYVWSSPSDLLELLVPGVSKKPSLSVQPGPVVAPGESLTLQCVSDVGYDRFVLYKEGERDLRQLPGRQPQAGLSQANFTLGPVSRSYGGQYRCYGAHNLSSECSAPSDPLDILITGQIRGTPFISVQPGPTVASGENVTLLCQSWRQFHTFLLTKAGAADAPLRLRSIHEYPKYQAEFPMSPVTSAHAGTYRCYGSLNSDPYLLSHPSEPLELVVS

SEQ ID NO: 6 - DNA, 1194 bp, synthetic construct
  misc_feature 1..1194: note = Variant V11 T71F
  source 1..1194: mol_type = other DNA
cagaccggcacaatcccaaagcccaccctgtgggccgagcctgattccgtgatcacccagggctctccagtgacactgtcctgccagggctctctggaggcccaggagtaccggctgtatagagagaagaagtctgccagctggatcttccggatcagacctgagctggtgaagaacggccagtttcacatcccaagcatcacctgggagcacacaggccggtacggatgccagtactattcccgggccagatggagcgagctgtccgaccctctggtgctggtcatgaccggcgcctatcctaagccaacactgagcgcccagccatcccctgtggtgaccagcggcggcagagtgacactgcagtgtgagtcccaggtggccttcggcggctttatcctgtgcaaggagggcgaggaggagcacccacagtgtctgaacagccagccacacgcccggggcagctccagagccatcttctccgtgggacccgtgagcccaaaccggagatggagccaccggtgctacggctatgacctgaatagcccttacgtgtggtctagcccatccgatctgctggagctgctggtgcccggcgtgtccaagaagccttccctgtctgtgcagccaggaccagtggtggcaccaggagagtctctgaccctgcagtgcgtgagcgacgtgggctacgatcggttcgtgctgtataaggagggagagagggatctgaggcagctgccaggcagacagccacaggccggcctgagccaggccaactttacactgggcccagtgagcaggtcctatggcggacagtacaggtgctatggagcacacaatctgtcctctgagtgttctgcccccagcgaccccctggacatcctgatcaccggccagatcaggggcacacccttcatctccgtgcagcctggaccaaccgtggcctctggcgagaacgtgacactgctgtgccagtcttggcgccagttccacacctttctgctgacaaaggcaggagcagcagacgcaccactgaggctgcgcagcatccacgagtaccccaagtatcaggccgagtttccaatgtctccagtgaccagcgcccacgcaggcacatacaggtgttatggcagcctgaacagcgacccctacctgctgagccacccttccgagccactggagctggtggtgagc

SEQ ID NO: 7 - protein, 398 aa, synthetic construct
  REGION 1..398: note = Variant V12 V78R
QTGTIPKPTLWAEPDSVITQGSPVTLSCQGSLEAQEYRLYREKKSASWITRIRPELRKNGQFHIPSITWEHTGRYGCQYYSRARWSELSDPLVLVMTGAYPKPTLSAQPSPVVTSGGRVTLQCESQVAFGGFILCKEGEEEHPQCLNSQPHARGSSRAIFSVGPVSPNRRWSHRCYGYDLNSPYVWSSPSDLLELLVPGVSKKPSLSVQPGPVVAPGESLTLQCVSDVGYDRFVLYKEGERDLRQLPGRQPQAGLSQANFTLGPVSRSYGGQYRCYGAHNLSSECSAPSDPLDILITGQIRGTPFISVQPGPTVASGENVTLLCQSWRQFHTFLLTKAGAADAPLRLRSIHEYPKYQAEFPMSPVTSAHAGTYRCYGSLNSDPYLLSHPSEPLELVVS

SEQ ID NO: 8 - DNA, 1194 bp, synthetic construct
  misc_feature 1..1194: note = Variant V12 V78R
  source 1..1194: mol_type = other DNA
cagaccggcacaatcccaaagcccaccctgtgggccgagcctgattccgtgatcacccagggctctccagtgacactgtcctgccagggctctctggaggcccaggagtaccggctgtatagagagaagaagtctgccagctggatcacccggatcagacctgagctgcggaagaacggccagtttcacatcccaagcatcacctgggagcacacaggccggtacggatgccagtactattcccgggccagatggagcgagctgtccgaccctctggtgctggtcatgaccggcgcctatcctaagccaacactgagcgcccagccatcccctgtggtgaccagcggcggcagagtgacactgcagtgtgagtcccaggtggccttcggcggctttatcctgtgcaaggagggcgaggaggagcacccacagtgtctgaacagccagccacacgcccggggcagctccagagccatcttctccgtgggacccgtgagcccaaaccggagatggagccaccggtgctacggctatgacctgaatagcccttacgtgtggtctagcccatccgatctgctggagctgctggtgcccggcgtgtccaagaagccttccctgtctgtgcagccaggaccagtggtggcaccaggagagtctctgaccctgcagtgcgtgagcgacgtgggctacgatcggttcgtgctgtataaggagggagagagggatctgaggcagctgccaggcagacagccacaggccggcctgagccaggccaactttacactgggcccagtgagcaggtcctatggcggacagtacaggtgctatggagcacacaatctgtcctctgagtgttctgcccccagcgaccccctggacatcctgatcaccggccagatcaggggcacacccttcatctccgtgcagcctggaccaaccgtggcctctggcgagaacgtgacactgctgtgccagtcttggcgccagttccacacctttctgctgacaaaggcaggagcagcagacgcaccactgaggctgcgcagcatccacgagtaccccaagtatcaggccgagtttccaatgtctccagtgaccagcgcccacgcaggcacatacaggtgttatggcagcctgaacagcgacccctacctgctgagccacccttccgagccactggagctggtggtgagc

SEQ ID NO: 9 - protein, 398 aa, synthetic construct
  REGION 1..398: note = Variant V13 S66Q
QTGTIPKPTLWAEPDSVITQGSPVTLSCQGSLEAQEYRLYREKKQASWITRIRPELVKNGQFHIPSITWEHTGRYGCQYYSRARWSELSDPLVLVMTGAYPKPTLSAQPSPVVTSGGRVTLQCESQVAFGGFILCKEGEEEHPQCLNSQPHARGSSRAIFSVGPVSPNRRWSHRCYGYDLNSPYVWSSPSDLLELLVPGVSKKPSLSVQPGPVVAPGESLTLQCVSDVGYDRFVLYKEGERDLRQLPGRQPQAGLSQANFTLGPVSRSYGGQYRCYGAHNLSSECSAPSDPLDILITGQIRGTPFISVQPGPTVASGENVTLLCQSWRQFHTFLLTKAGAADAPLRLRSIHEYPKYQAEFPMSPVTSAHAGTYRCYGSLNSDPYLLSHPSEPLELVVS

SEQ ID NO: 10 - DNA, 1194 bp, synthetic construct
  misc_feature 1..1194: note = Variant V13 S66Q
  source 1..1194: mol_type = other DNA
cagaccggcacaatcccaaagcccaccctgtgggccgagcctgattccgtgatcacccagggctctccagtgacactgtcctgccagggctctctggaggcccaggagtaccggctgtatagagagaagaagcaggccagctggatcacccggatcagacctgagctggtgaagaacggccagtttcacatcccaagcatcacctgggagcacacaggccggtacggatgccagtactattcccgggccagatggagcgagctgtccgaccctctggtgctggtcatgaccggcgcctatcctaagccaacactgagcgcccagccatcccctgtggtgaccagcggcggcagagtgacactgcagtgtgagtcccaggtggccttcggcggctttatcctgtgcaaggagggcgaggaggagcacccacagtgtctgaacagccagccacacgcccggggcagctccagagccatcttctccgtgggacccgtgagcccaaaccggagatggagccaccggtgctacggctatgacctgaatagcccttacgtgtggtctagcccatccgatctgctggagctgctggtgcccggcgtgtccaagaagccttccctgtctgtgcagccaggaccagtggtggcaccaggagagtctctgaccctgcagtgcgtgagcgacgtgggctacgatcggttcgtgctgtataaggagggagagagggatctgaggcagctgccaggcagacagccacaggccggcctgagccaggccaactttacactgggcccagtgagcaggtcctatggcggacagtacaggtgctatggagcacacaatctgtcctctgagtgttctgcccccagcgaccccctggacatcctgatcaccggccagatcaggggcacacccttcatctccgtgcagcctggaccaaccgtggcctctggcgagaacgtgacactgctgtgccagtcttggcgccagttccacacctttctgctgacaaaggcaggagcagcagacgcaccactgaggctgcgcagcatccacgagtaccccaagtatcaggccgagtttccaatgtctccagtgaccagcgcccacgcaggcacatacaggtgttatggcagcctgaacagcgacccctacctgctgagccacccttccgagccactggagctggtggtgagc

SEQ ID NO: 11 - protein, 398 aa, synthetic construct
  REGION 1..398: note = Variant V14 I70K
QTGTIPKPTLWAEPDSVITQGSPVTLSCQGSLEAQEYRLYREKKSASWKTRIRPELVKNGQFHIPSITWEHTGRYGCQYYSRARWSELSDPLVLVMTGAYPKPTLSAQPSPVVTSGGRVTLQCESQVAFGGFILCKEGEEEHPQCLNSQPHARGSSRAIFSVGPVSPNRRWSHRCYGYDLNSPYVWSSPSDLLELLVPGVSKKPSLSVQPGPVVAPGESLTLQCVSDVGYDRFVLYKEGERDLRQLPGRQPQAGLSQANFTLGPVSRSYGGQYRCYGAHNLSSECSAPSDPLDILITGQIRGTPFISVQPGPTVASGENVTLLCQSWRQFHTFLLTKAGAADAPLRLRSIHEYPKYQAEFPMSPVTSAHAGTYRCYGSLNSDPYLLSHPSEPLELVVS

SEQ ID NO: 12 - DNA, 1194 bp, synthetic construct
  misc_feature 1..1194: note = Variant V14 I70K
  source 1..1194: mol_type = other DNA
cagaccggcacaatcccaaagcccaccctgtgggccgagcctgattccgtgatcacccagggctctccagtgacactgtcctgccagggctctctggaggcccaggagtaccggctgtatagagagaagaagtctgccagctggaagacccggatcagacctgagctggtgaagaacggccagtttcacatcccaagcatcacctgggagcacacaggccggtacggatgccagtactattcccgggccagatggagcgagctgtccgaccctctggtgctggtcatgaccggcgcctatcctaagccaacactgagcgcccagccatcccctgtggtgaccagcggcggcagagtgacactgcagtgtgagtcccaggtggccttcggcggctttatcctgtgcaaggagggcgaggaggagcacccacagtgtctgaacagccagccacacgcccggggcagctccagagccatcttctccgtgggacccgtgagcccaaaccggagatggagccaccggtgctacggctatgacctgaatagcccttacgtgtggtctagcccatccgatctgctggagctgctggtgcccggcgtgtccaagaagccttccctgtctgtgcagccaggaccagtggtggcaccaggagagtctctgaccctgcagtgcgtgagcgacgtgggctacgatcggttcgtgctgtataaggagggagagagggatctgaggcagctgccaggcagacagccacaggccggcctgagccaggccaactttacactgggcccagtgagcaggtcctatggcggacagtacaggtgctatggagcacacaatctgtcctctgagtgttctgcccccagcgaccccctggacatcctgatcaccggccagatcaggggcacacccttcatctccgtgcagcctggaccaaccgtggcctctggcgagaacgtgacactgctgtgccagtcttggcgccagttccacacctttctgctgacaaaggcaggagcagcagacgcaccactgaggctgcgcagcatccacgagtaccccaagtatcaggccgagtttccaatgtctccagtgaccagcgcccacgcaggcacatacaggtgttatggcagcctgaacagcgacccctacctgctgagccacccttccgagccactggagctggtggtgagc

SEQ ID NO: 13 - protein, 398 aa, synthetic construct
  REGION 1..398: note = Variant V15 S66Q I70K
QTGTIPKPTLWAEPDSVITQGSPVTLSCQGSLEAQEYRLYREKKQASWKTRIRPELVKNGQFHIPSITWEHTGRYGCQYYSRARWSELSDPLVLVMTGAYPKPTLSAQPSPVVTSGGRVTLQCESQVAFGGFILCKEGEEEHPQCLNSQPHARGSSRAIFSVGPVSPNRRWSHRCYGYDLNSPYVWSSPSDLLELLVPGVSKKPSLSVQPGPVVAPGESLTLQCVSDVGYDRFVLYKEGERDLRQLPGRQPQAGLSQANFTLGPVSRSYGGQYRCYGAHNLSSECSAPSDPLDILITGQIRGTPFISVQPGPTVASGENVTLLCQSWRQFHTFLLTKAGAADAPLRLRSIHEYPKYQAEFPMSPVTSAHAGTYRCYGSLNSDPYLLSHPSEPLELVVS

SEQ ID NO: 14 - DNA, 1194 bp, synthetic construct
  misc_feature 1..1194: note = Variant V15 S66Q I70K
  source 1..1194: mol_type = other DNA
cagaccggcacaatcccaaagcccaccctgtgggccgagcctgattccgtgatcacccagggctctccagtgacactgtcctgccagggctctctggaggcccaggagtaccggctgtatagagagaagaagcaggccagctggaagacccggatcagacctgagctggtgaagaacggccagtttcacatcccaagcatcacctgggagcacacaggccggtacggatgccagtactattcccgggccagatggagcgagctgtccgaccctctggtgctggtcatgaccggcgcctatcctaagccaacactgagcgcccagccatcccctgtggtgaccagcggcggcagagtgacactgcagtgtgagtcccaggtggccttcggcggctttatcctgtgcaaggagggcgaggaggagcacccacagtgtctgaacagccagccacacgcccggggcagctccagagccatcttctccgtgggacccgtgagcccaaaccggagatggagccaccggtgctacggctatgacctgaatagcccttacgtgtggtctagcccatccgatctgctggagctgctggtgcccggcgtgtccaagaagccttccctgtctgtgcagccaggaccagtggtggcaccaggagagtctctgaccctgcagtgcgtgagcgacgtgggctacgatcggttcgtgctgtataaggagggagagagggatctgaggcagctgccaggcagacagccacaggccggcctgagccaggccaactttacactgggcccagtgagcaggtcctatggcggacagtacaggtgctatggagcacacaatctgtcctctgagtgttctgcccccagcgaccccctggacatcctgatcaccggccagatcaggggcacacccttcatctccgtgcagcctggaccaaccgtggcctctggcgagaacgtgacactgctgtgccagtcttggcgccagttccacacctttctgctgacaaaggcaggagcagcagacgcaccactgaggctgcgcagcatccacgagtaccccaagtatcaggccgagtttccaatgtctccagtgaccagcgcccacgcaggcacatacaggtgttatggcagcctgaacagcgacccctacctgctgagccacccttccgagccactggagctggtggtgagc

SEQ ID NO: 15 - protein, 398 aa, synthetic construct
  REGION 1..398: note = Variant V16 S66Q V78R
QTGTIPKPTLWAEPDSVITQGSPVTLSCQGSLEAQEYRLYREKKQASWITRIRPELRKNGQFHIPSITWEHTGRYGCQYYSRARWSELSDPLVLVMTGAYPKPTLSAQPSPVVTSGGRVTLQCESQVAFGGFILCKEGEEEHPQCLNSQPHARGSSRAIFSVGPVSPNRRWSHRCYGYDLNSPYVWSSPSDLLELLVPGVSKKPSLSVQPGPVVAPGESLTLQCVSDVGYDRFVLYKEGERDLRQLPGRQPQAGLSQANFTLGPVSRSYGGQYRCYGAHNLSSECSAPSDPLDILITGQIRGTPFISVQPGPTVASGENVTLLCQSWRQFHTFLLTKAGAADAPLRLRSIHEYPKYQAEFPMSPVTSAHAGTYRCYGSLNSDPYLLSHPSEPLELVVS

SEQ ID NO: 16 - DNA, 1194 bp, synthetic construct
  misc_feature 1..1194: note = Variant V16 S66Q V78R
  source 1..1194: mol_type = other DNA
cagaccggcacaatcccaaagcccaccctgtgggccgagcctgattccgtgatcacccagggctctccagtgacactgtcctgccagggctctctggaggcccaggagtaccggctgtatagagagaagaagcaggccagctggatcacccggatcagacctgagctgagaaagaacggccagtttcacatcccaagcatcacctgggagcacacaggccggtacggatgccagtactattcccgggccagatggagcgagctgtccgaccctctggtgctggtcatgaccggcgcctatcctaagccaacactgagcgcccagccatcccctgtggtgaccagcggcggcagagtgacactgcagtgtgagtcccaggtggccttcggcggctttatcctgtgcaaggagggcgaggaggagcacccacagtgtctgaacagccagccacacgcccggggcagctccagagccatcttctccgtgggacccgtgagcccaaaccggagatggagccaccggtgctacggctatgacctgaatagcccttacgtgtggtctagcccatccgatctgctggagctgctggtgcccggcgtgtccaagaagccttccctgtctgtgcagccaggaccagtggtggcaccaggagagtctctgaccctgcagtgcgtgagcgacgtgggctacgatcggttcgtgctgtataaggagggagagagggatctgaggcagctgccaggcagacagccacaggccggcctgagccaggccaactttacactgggcccagtgagcaggtcctatggcggacagtacaggtgctatggagcacacaatctgtcctctgagtgttctgcccccagcgaccccctggacatcctgatcaccggccagatcaggggcacacccttcatctccgtgcagcctggaccaaccgtggcctctggcgagaacgtgacactgctgtgccagtcttggcgccagttccacacctttctgctgacaaaggcaggagcagcagacgcaccactgaggctgcgcagcatccacgagtaccccaagtatcaggccgagtttccaatgtctccagtgaccagcgcccacgcaggcacatacaggtgttatggcagcctgaacagcgacccctacctgctgagccacccttccgagccactggagctggtggtgagc

SEQ ID NO: 17 - protein, 398 aa, synthetic construct
  REGION 1..398: note = Variant V17 S66R I70F
QTGTIPKPTLWAEPDSVITQGSPVTLSCQGSLEAQEYRLYREKKRASWFTRIRPELVKNGQFHIPSITWEHTGRYGCQYYSRARWSELSDPLVLVMTGAYPKPTLSAQPSPVVTSGGRVTLQCESQVAFGGFILCKEGEEEHPQCLNSQPHARGSSRAIFSVGPVSPNRRWSHRCYGYDLNSPYVWSSPSDLLELLVPGVSKKPSLSVQPGPVVAPGESLTLQCVSDVGYDRFVLYKEGERDLRQLPGRQPQAGLSQANFTLGPVSRSYGGQYRCYGAHNLSSECSAPSDPLDILITGQIRGTPFISVQPGPTVASGENVTLLCQSWRQFHTFLLTKAGAADAPLRLRSIHEYPKYQAEFPMSPVTSAHAGTYRCYGSLNSDPYLLSHPSEPLELVVS

SEQ ID NO: 18 - DNA, 1194 bp, synthetic construct
  misc_feature 1..1194: note = Variant V17 S66R I70F
  source 1..1194: mol_type = other DNA
cagaccggcacaatcccaaagcccaccctgtgggccgagcctgattccgtgatcacccagggctctccagtgacactgtcctgccagggctctctggaggcccaggagtaccggctgtatagagagaagaagagagccagctggttcacccggatcagacctgagctggtgaagaacggccagtttcacatcccaagcatcacctgggagcacacaggccggtacggatgccagtactattcccgggccagatggagcgagctgtccgaccctctggtgctggtcatgaccggcgcctatcctaagccaacactgagcgcccagccatcccctgtggtgaccagcggcggcagagtgacactgcagtgtgagtcccaggtggccttcggcggctttatcctgtgcaaggagggcgaggaggagcacccacagtgtctgaacagccagccacacgcccggggcagctccagagccatcttctccgtgggacccgtgagcccaaaccggagatggagccaccggtgctacggctatgacctgaatagcccttacgtgtggtctagcccatccgatctgctggagctgctggtgcccggcgtgtccaagaagccttccctgtctgtgcagccaggaccagtggtggcaccaggagagtctctgaccctgcagtgcgtgagcgacgtgggctacgatcggttcgtgctgtataaggagggagagagggatctgaggcagctgccaggcagacagccacaggccggcctgagccaggccaactttacactgggcccagtgagcaggtcctatggcggacagtacaggtgctatggagcacacaatctgtcctctgagtgttctgcccccagcgaccccctggacatcctgatcaccggccagatcaggggcacacccttcatctccgtgcagcctggaccaaccgtggcctctggcgagaacgtgacactgctgtgccagtcttggcgccagttccacacctttctgctgacaaaggcaggagcagcagacgcaccactgaggctgcgcagcatccacgagtaccccaagtatcaggccgagtttccaatgtctccagtgaccagcgcccacgcaggcacatacaggtgttatggcagcctgaacagcgacccctacctgctgagccacccttccgagccactggagctggtggtgagc

SEQ ID NO: 19 - protein, 398 aa, synthetic construct
  REGION 1..398: note = Variant V18 S66Y T71K
QTGTIPKPTLWAEPDSVITQGSPVTLSCQGSLEAQEYRLYREKKYASWIKRIRPELVKNGQFHIPSITWEHTGRYGCQYYSRARWSELSDPLVLVMTGAYPKPTLSAQPSPVVTSGGRVTLQCESQVAFGGFILCKEGEEEHPQCLNSQPHARGSSRAIFSVGPVSPNRRWSHRCYGYDLNSPYVWSSPSDLLELLVPGVSKKPSLSVQPGPVVAPGESLTLQCVSDVGYDRFVLYKEGERDLRQLPGRQPQAGLSQANFTLGPVSRSYGGQYRCYGAHNLSSECSAPSDPLDILITGQIRGTPFISVQPGPTVASGENVTLLCQSWRQFHTFLLTKAGAADAPLRLRSIHEYPKYQAEFPMSPVTSAHAGTYRCYGSLNSDPYLLSHPSEPLELVVS

SEQ ID NO: 20 - DNA, 1194 bp, synthetic construct
  misc_feature 1..1194: note = Variant V18 S66Y T71K
  source 1..1194: mol_type = other DNA
cagaccggcacaatcccaaagcccaccctgtgggccgagcctgattccgtgatcacccagggctctccagtgacactgtcctgccagggctctctggaggcccaggagtaccggctgtatagagagaagaagtacgccagctggatcaagcggatcagacctgagctggtgaagaacggccagtttcacatcccaagcatcacctgggagcacacaggccggtacggatgccagtactattcccgggccagatggagcgagctgtccgaccctctggtgctggtcatgaccggcgcctatcctaagccaacactgagcgcccagccatcccctgtggtgaccagcggcggcagagtgacactgcagtgtgagtcccaggtggccttcggcggctttatcctgtgcaaggagggcgaggaggagcacccacagtgtctgaacagccagccacacgcccggggcagctccagagccatcttctccgtgggacccgtgagcccaaaccggagatggagccaccggtgctacggctatgacctgaatagcccttacgtgtggtctagcccatccgatctgctggagctgctggtgcccggcgtgtccaagaagccttccctgtctgtgcagccaggaccagtggtggcaccaggagagtctctgaccctgcagtgcgtgagcgacgtgggctacgatcggttcgtgctgtataaggagggagagagggatctgaggcagctgccaggcagacagccacaggccggcctgagccaggccaactttacactgggcccagtgagcaggtcctatggcggacagtacaggtgctatggagcacacaatctgtcctctgagtgttctgcccccagcgaccccctggacatcctgatcaccggccagatcaggggcacacccttcatctccgtgcagcctggaccaaccgtggcctctggcgagaacgtgacactgctgtgccagtcttggcgccagttccacacctttctgctgacaaaggcaggagcagcagacgcaccactgaggctgcgcagcatccacgagtaccccaagtatcaggccgagtttccaatgtctccagtgaccagcgcccacgcaggcacatacaggtgttatggcagcctgaacagcgacccctacctgctgagccacccttccgagccactggagctggtggtgagc

SEQ ID NO: 21 - protein, 398 aa, synthetic construct
  REGION 1..398: note = Variant V19 S66Y T71N
QTGTIPKPTLWAEPDSVITQGSPVTLSCQGSLEAQEYRLYREKKYASWINRIRPELVKNGQFHIPSITWEHTGRYGCQYYSRARWSELSDPLVLVMTGAYPKPTLSAQPSPVVTSGGRVTLQCESQVAFGGFILCKEGEEEHPQCLNSQPHARGSSRAIFSVGPVSPNRRWSHRCYGYDLNSPYVWSSPSDLLELLVPGVSKKPSLSVQPGPVVAPGESLTLQCVSDVGYDRFVLYKEGERDLRQLPGRQPQAGLSQANFTLGPVSRSYGGQYRCYGAHNLSSECSAPSDPLDILITGQIRGTPFISVQPGPTVASGENVTLLCQSWRQFHTFLLTKAGAADAPLRLRSIHEYPKYQAEFPMSPVTSAHAGTYRCYGSLNSDPYLLSHPSEPLELVVS

SEQ ID NO: 22 - DNA, 1196 bp, synthetic construct
  misc_feature 1..1196: note = Variant V19 S66Y T71N
  misc_feature 1: note = n is a, c, g, or t
  source 1..1196: mol_type = other DNA
nacagaccggcacaatcccaaagcccaccctgtgggccgagcctgattccgtgatcacccagggctctccagtgacactgtcctgccagggctctctggaggcccaggagtaccggctgtatagagagaagaagtacgccagctggatcaaccggatcagacctgagctggtgaagaacggccagtttcacatcccaagcatcacctgggagcacacaggccggtacggatgccagtactattcccgggccagatggagcgagctgtccgaccctctggtgctggtcatgaccggcgcctatcctaagccaacactgagcgcccagccatcccctgtggtgaccagcggcggcagagtgacactgcagtgtgagtcccaggtggccttcggcggctttatcctgtgcaaggagggcgaggaggagcacccacagtgtctgaacagccagccacacgcccggggcagctccagagccatcttctccgtgggacccgtgagcccaaaccggagatggagccaccggtgctacggctatgacctgaatagcccttacgtgtggtctagcccatccgatctgctggagctgctggtgcccggcgtgtccaagaagccttccctgtctgtgcagccaggaccagtggtggcaccaggagagtctctgaccctgcagtgcgtgagcgacgtgggctacgatcggttcgtgctgtataaggagggagagagggatctgaggcagctgccaggcagacagccacaggccggcctgagccaggccaactttacactgggcccagtgagcaggtcctatggcggacagtacaggtgctatggagcacacaatctgtcctctgagtgttctgcccccagcgaccccctggacatcctgatcaccggccagatcaggggcacacccttcatctccgtgcagcctggaccaaccgtggcctctggcgagaacgtgacactgctgtgccagtcttggcgccagttccacacctttctgctgacaaaggcaggagcagcagacgcaccactgaggctgcgcagcatccacgagtaccccaagtatcaggccgagtttccaatgtctccagtgaccagcgcccacgcaggcacatacaggtgttatggcagcctgaacagcgacccctacctgctgagccacccttccgagccactggagctggtggtgagc

SEQ ID NO: 23 - protein, 398 aa, synthetic construct
  REGION 1..398: note = Variant V20 S66N T71R
QTGTIPKPTLWAEPDSVITQGSPVTLSCQGSLEAQEYRLYREKKNASWIRRIRPELVKNGQFHIPSITWEHTGRYGCQYYSRARWSELSDPLVLVMTGAYPKPTLSAQPSPVVTSGGRVTLQCESQVAFGGFILCKEGEEEHPQCLNSQPHARGSSRAIFSVGPVSPNRRWSHRCYGYDLNSPYVWSSPSDLLELLVPGVSKKPSLSVQPGPVVAPGESLTLQCVSDVGYDRFVLYKEGERDLRQLPGRQPQAGLSQANFTLGPVSRSYGGQYRCYGAHNLSSECSAPSDPLDILITGQIRGTPFISVQPGPTVASGENVTLLCQSWRQFHTFLLTKAGAADAPLRLRSIHEYPKYQAEFPMSPVTSAHAGTYRCYGSLNSDPYLLSHPSEPLELVVS

SEQ ID NO: 24 - DNA, 1194 bp, synthetic construct
  misc_feature 1..1194: note = Variant V20 S66N T71R
  source 1..1194: mol_type = other DNA
cagaccggcacaatcccaaagcccaccctgtgggccgagcctgattccgtgatcacccagggctctccagtgacactgtcctgccagggctctctggaggcccaggagtaccggctgtatagagagaagaagaacgccagctggatcagacggatcagacctgagctggtgaagaacggccagtttcacatcccaagcatcacctgggagcacacaggccggtacggatgccagtactattcccgggccagatggagcgagctgtccgaccctctggtgctggtcatgaccggcgcctatcctaagccaacactgagcgcccagccatcccctgtggtgaccagcggcggcagagtgacactgcagtgtgagtcccaggtggccttcggcggctttatcctgtgcaaggagggcgaggaggagcacccacagtgtctgaacagccagccacacgcccggggcagctccagagccatcttctccgtgggacccgtgagcccaaaccggagatggagccaccggtgctacggctatgacctgaatagcccttacgtgtggtctagcccatccgatctgctggagctgctggtgcccggcgtgtccaagaagccttccctgtctgtgcagccaggaccagtggtggcaccaggagagtctctgaccctgcagtgcgtgagcgacgtgggctacgatcggttcgtgctgtataaggagggagagagggatctgaggcagctgccaggcagacagccacaggccggcctgagccaggccaactttacactgggcccagtgagcaggtcctatggcggacagtacaggtgctatggagcacacaatctgtcctctgagtgttctgcccccagcgaccccctggacatcctgatcaccggccagatcaggggcacacccttcatctccgtgcagcctggaccaaccgtggcctctggcgagaacgtgacactgctgtgccagtcttggcgccagttccacacctttctgctgacaaaggcaggagcagcagacgcaccactgaggctgcgcagcatccacgagtaccccaagtatcaggccgagtttccaatgtctccagtgaccagcgcccacgcaggcacatacaggtgttatggcagcctgaacagcgacccctacctgctgagccacccttccgagccactggagctggtggtgagc

SEQ ID NO: 25 - protein, 21 aa, synthetic construct
  REGION 1..21: note = Signal peptide aa
MESPAQLLFLLLLWLPDGVHA

SEQ ID NO: 26 - DNA, 63 bp, synthetic construct
  misc_feature 1..63: note = Signal peptide NA
  source 1..63: mol_type = other DNA
atggagtccccagcacagctgctgttcctgctgctgctgtggctgcctgacggagtgcacgca

SEQ ID NO: 27 - protein, 343 aa, synthetic construct
  REGION 1..343: note = SIRPa AA
EEELQVIQPDKSVLVAAGETATLRCTATSLIPVGPIQWFRGAGPGRELIYNQKEGHFPRVTTVSDLTKRNNMDFSIRIGNITPADAGTYYCVKFRKGSPDDVEFKSGAGTELSVRAKPSAPVVSGPAARATPQHTVSFTCESHGFSPRDITLKWFKNGNELSDFQTNVDPVGESVSYSIHSTAKVVLTREDVHSQVICEVAHVTLQGDPLRGTANLSETIRVPPTLEVTQQPVRAENQVNVTCQVRKFYPQRLQLTWLENGNVSRTETASTVTENKDGTYNWMSWLLVNVSAHRDDVKLTCQVEHDGQPAVSKSHDLKVSAHPKEQGSNTAAENTGSNERNIY

SEQ ID NO: 28 - DNA, 1029 bp, synthetic construct
  misc_feature 1..1029: note = SIRPa NA
  source 1..1029: mol_type = other DNA
gaggaggagctgcaggtcatccagcccgataagtctgtgctggtggcagcaggagagaccgccacactgaggtgcaccgccacaagcctgatcccagtgggaccaatccagtggtttaggggagcaggccctggcagagagctgatctacaaccagaaggagggccacttcccaagagtgaccacagtgagcgacctgaccaagcggaacaatatggatttttccatcagaatcggcaatatcacacctgccgacgccggcacctactattgcgtgaagttcaggaagggctccccagacgatgtggagtttaagagcggagcaggcaccgagctgtccgtgcgggcaaagccttccgccccagtggtgtctggaccagcagccagagccaccccacagcacacagtgtccttcacctgtgagtctcacggctttagcccccgggacatcaccctgaagtggttcaagaacggcaatgagctgtctgactttcagaccaacgtggaccccgtgggcgagtctgtgagctattccatccactctacagccaaggtggtgctgacccgcgaggacgtgcacagccaggtcatctgcgaggtggcacacgtgaccctgcagggcgatcctctgaggggcacagccaatctgagcgagaccatcagagtgccccctacactggaggtgacccagcagcccgtgcgcgcagagaaccaagtgaatgtgacatgtcaggtgaggaagttctaccctcagcgcctgcagctgacctggctggagaacggcaacgtgagccggaccgagacagccagcaccgtgacagagaacaaggacggcacatataattggatgtcttggctgctggtgaacgtgagcgcccacagggacgatgtgaagctgacctgccaggtggagcacgacggacagccagccgtgtctaagagccacgatctgaaggtgagcgcccaccctaaggagcagggctccaacacagccgccgagaataccggcagcaacgagcggaatatctac

SEQ ID NO: 29 - protein, 232 aa, synthetic construct
  REGION 1..232: note = Fc IgG1 "Hole" LALA, Fc (AA 99-330) P01857-1 C220S L234A, L235Aplus "Hole" T366S (249) and L368A (251) Y407V (290) and Y349C(232)
EPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 30 - DNA, 696 bp, synthetic construct
  misc_feature 1..696: note = Fc IgG1 "Hole" LALA, NA
  source 1..696: mol_type = other DNA
gagcctaagagctccgacaagacccacacatgcccaccttgtccagcacctgaggcagcaggaggaccatccgtgttcctgtttccacccaagcctaaggataccctgatgatctctcgcacccctgaggtgacatgcgtggtggtggacgtgagccacgaggaccccgaggtgaagtttaactggtacgtggacggcgtggaggtgcacaatgccaagacaaagccccgggaggagcagtacaacagcacctatagagtggtgtccgtgctgacagtgctgcaccaggattggctgaacggcaaggagtacaagtgtaaggtgtccaataaggccctgccagcccccatcgagaagaccatctctaaggcaaagggacagcccagggagcctcaggtgtgcaccctgcctccaagccgcgacgagctgacaaagaaccaggtgtctctgagctgtgccgtgaagggcttctacccatctgacatcgccgtggagtgggagagcaatggccagcccgagaacaattataagaccacaccccctgtgctggactctgatggcagcttctttctggtgtccaagctgaccgtggataagtctaggtggcagcagggcaacgtgttttcctgttctgtgatgcacgaggccctgcacaatcactacacacagaagagcctgtccctgtctcccggcaag

SEQ ID NO: 31 - protein, 233 aa, synthetic construct
  REGION 1..233: note = Fc IgG1 "knob"LALA (Fc IgG1 (AA 99-330) P01857-1 C220S (kabat)plus "Knob" T366W (kabat) and S354C (kabat) plus L234A, L235A(kabat)
SEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 32 - DNA, 699 bp, synthetic construct
  misc_feature 1..699: note = Fc IgG1 knob NA
  source 1..699: mol_type = other DNA
tccgagcctaagagctccgacaagacccacacatgcccaccatgtcctgcaccagaggcagcaggaggaccttccgtgttcctgtttcctccaaagccaaaggatacactgatgatctccagaacaccagaggtgacctgcgtggtggtggacgtgtctcacgaggaccccgaggtgaagtttaactggtacgtggacggcgtggaggtgcacaatgccaagaccaagccaagggaggagcagtacaactccacatatcgcgtggtgtctgtgctgaccgtgctgcaccaggattggctgaacggcaaggagtataagtgtaaggtgagcaataaggccctgcccgcccctatcgagaagaccatctccaaggcaaagggacagcccagggagcctcaggtgtacacactgcccccttgccgcgacgagctgaccaagaaccaggtgtctctgtggtgtctggtgaagggcttctacccatctgacatcgccgtggagtgggagagcaatggccagcccgagaacaattacaagaccacaccacccgtgctggacagcgatggctccttctttctgtattccaagctgacagtggacaagtctcggtggcagcagggcaacgtgttttcctgttctgtgatgcacgaggccctgcacaatcactatacccagaagagcctgtccctgtctcccggcaag

SEQ ID NO: 33 - protein, 10 aa, synthetic construct
  REGION 1..10: note = Linker (between SIRPa and Fc)
GGGGSGGGGS

SEQ ID NO: 34 - protein, 30 aa, synthetic construct
  REGION 1..30: note = Linker NA
GGAGGAGGAGGCTCCGGAGGAGGAGGCTCT

SEQ ID NO: 35 - protein, 585 aa, synthetic construct
  REGION 1..585: note = SIRPa fused to Fc of IgG1 "knob" LALA AA,
EEELQVIQPDKSVLVAAGETATLRCTATSLIPVGPIQWFRGAGPGRELIYNQKEGHFPRVTTVSDLTKRNNMDFSIRIGNITPADAGTYYCVKFRKGSPDDVEFKSGAGTELSVRAKPSAPVVSGPAARATPQHTVSFTCESHGFSPRDITLKWFKNGNELSDFQTNVDPVGESVSYSIHSTAKVVLTREDVHSQVICEVAHVTLQGDPLRGTANLSETIRVPPTLEVTQQPVRAENQVNVTCQVRKFYPQRLQLTWLENGNVSRTETASTVTENKDGTYNWMSWLLVNVSAHRDDVKLTCQVEHDGQPAVSKSHDLKVSAHPKEQGSNTAAENTGSNERNIYGGGGSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 36 - DNA, 1755 bp, synthetic construct
  misc_feature 1..1755: note = SIRPa fused to Fc of IgG1 "knob" LALA NA,
  source 1..1755: mol_type = other DNA
gaggaggagctgcaggtcatccagcccgataagtctgtgctggtggcagcaggagagaccgccacactgaggtgcaccgccacaagcctgatcccagtgggaccaatccagtggtttaggggagcaggccctggcagagagctgatctacaaccagaaggagggccacttcccaagagtgaccacagtgagcgacctgaccaagcggaacaatatggatttttccatcagaatcggcaatatcacacctgccgacgccggcacctactattgcgtgaagttcaggaagggctccccagacgatgtggagtttaagagcggagcaggcaccgagctgtccgtgcgggcaaagccttccgccccagtggtgtctggaccagcagccagagccaccccacagcacacagtgtccttcacctgtgagtctcacggctttagcccccgggacatcaccctgaagtggttcaagaacggcaatgagctgtctgactttcagaccaacgtggaccccgtgggcgagtctgtgagctattccatccactctacagccaaggtggtgctgacccgcgaggacgtgcacagccaggtcatctgcgaggtggcacacgtgaccctgcagggcgatcctctgaggggcacagccaatctgagcgagaccatcagagtgccccctacactggaggtgacccagcagcccgtgcgcgcagagaaccaagtgaatgtgacatgtcaggtgaggaagttctaccctcagcgcctgcagctgacctggctggagaacggcaacgtgagccggaccgagacagccagcaccgtgacagagaacaaggacggcacatataattggatgtcttggctgctggtgaacgtgagcgcccacagggacgatgtgaagctgacctgccaggtggagcacgacggacagccagccgtgtctaagagccacgatctgaaggtgagcgcccaccctaaggagcagggctccaacacagccgccgagaataccggcagcaacgagcggaatatctacggaggaggaggcagcggaggaggaggctccgagcctaagagctccgacaagacccacacatgcccaccatgtcctgcaccagaggcagcaggaggaccttccgtgttcctgtttcctccaaagccaaaggatacactgatgatctccagaacaccagaggtgacctgcgtggtggtggacgtgtctcacgaggaccccgaggtgaagtttaactggtacgtggacggcgtggaggtgcacaatgccaagaccaagccaagggaggagcagtacaactccacatatcgcgtggtgtctgtgctgaccgtgctgcaccaggattggctgaacggcaaggagtataagtgtaaggtgagcaataaggccctgcccgcccctatcgagaagaccatctccaaggcaaagggacagcccagggagcctcaggtgtacacactgcccccttgccgcgacgagctgaccaagaaccaggtgtctctgtggtgtctggtgaagggcttctacccatctgacatcgccgtggagtgggagagcaatggccagcccgagaacaattacaagaccacaccacccgtgctggacagcgatggctccttctttctgtattccaagctgacagtggacaagtctcggtggcagcagggcaacgtgttttcctgttctgtgatgcacgaggccctgcacaatcactatacccagaagagcctgtccctgtctcccggcaag

SEQ ID NO: 37 - protein, 645 aa, synthetic construct
  REGION 1..645: note = designed LILRB2 variants
QTGTIPKPTLWAEPDSVITQGSPVTLSCQGSLEAQEYRLYREKKSASWITRIRPELVKNGQFHIPSITWEHTGRYGCQYYSRARWSELSDPLVLVMTGAYPKPTLSAQPSPVVTSGGRVTLQCESQVAFGGFILCKEGEEEHPQCLNSQPHARGSSRAIFSVGPVSPNRRWSHRCYGYDLNSPYVWSSPSDLLELLVPGVSKKPSLSVQPGPVVAPGESLTLQCVSDVGYDRFVLYKEGERDLRQLPGRQPQAGLSQANFTLGPVSRSYGGQYRCYGAHNLSSECSAPSDPLDILITGQIRGTPFISVQPGPTVASGENVTLLCQSWRQFHTFLLTKAGAADAPLRLRSIHEYPKYQAEFPMSPVTSAHAGTYRCYGSLNSDPYLLSHPSEPLELVVSGGGGSGGGGSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 38 - DNA, 1944 bp, synthetic construct
  misc_feature 1..1944: note = designed LILRB2 variants
  source 1..1944: mol_type = other DNA
cagaccggcacaatcccaaagcccaccctgtgggccgagcctgattccgtgatcacccagggctctccagtgacactgtcctgccagggctctctggaggcccaggagtaccggctgtatagagagaagaagtctgccagctggatcacccggatcagacctgagctggtgaagaacggccagtttcacatcccaagcatcacctgggagcacacaggccggtacggatgccagtactattcccgggccagatggagcgagctgtccgaccctctggtgctggtcatgaccggcgcctatcctaagccaacactgagcgcccagccatcccctgtggtgaccagcggcggcagagtgacactgcagtgtgagtcccaggtggccttcggcggctttatcctgtgcaaggagggcgaggaggagcacccacagtgtctgaacagccagccacacgcccggggcagctccagagccatcttctccgtgggacccgtgagcccaaaccggagatggagccaccggtgctacggctatgacctgaatagcccttacgtgtggtctagcccatccgatctgctggagctgctggtgcccggcgtgtccaagaagccttccctgtctgtgcagccaggaccagtggtggcaccaggagagtctctgaccctgcagtgcgtgagcgacgtgggctacgatcggttcgtgctgtataaggagggagagagggatctgaggcagctgccaggcagacagccacaggccggcctgagccaggccaactttacactgggcccagtgagcaggtcctatggcggacagtacaggtgctatggagcacacaatctgtcctctgagtgttctgcccccagcgaccccctggacatcctgatcaccggccagatcaggggcacacccttcatctccgtgcagcctggaccaaccgtggcctctggcgagaacgtgacactgctgtgccagtcttggcgccagttccacacctttctgctgacaaaggcaggagcagcagacgcaccactgaggctgcgcagcatccacgagtaccccaagtatcaggccgagtttccaatgtctccagtgaccagcgcccacgcaggcacatacaggtgttatggcagcctgaacagcgacccctacctgctgagccacccttccgagccactggagctggtggtgagcggaggaggaggctccggaggaggaggctctggcggcggcggcagcgagcctaagagctccgacaagacccacacatgcccaccttgtccagcacctgaggcagcaggaggaccatccgtgttcctgtttccacccaagcctaaggataccctgatgatctctcgcacccctgaggtgacatgcgtggtggtggacgtgagccacgaggaccccgaggtgaagtttaactggtacgtggacggcgtggaggtgcacaatgccaagacaaagccccgggaggagcagtacaacagcacctatagagtggtgtccgtgctgacagtgctgcaccaggattggctgaacggcaaggagtacaagtgtaaggtgtccaataaggccctgccagcccccatcgagaagaccatctctaaggcaaagggacagcccagggagcctcaggtgtgcaccctgcctccaagccgcgacgagctgacaaagaaccaggtgtctctgagctgtgccgtgaagggcttctacccatctgacatcgccgtggagtgggagagcaatggccagcccgagaacaattataagaccacaccccctgtgctggactctgatggcagcttctttctggtgtccaagctgaccgtggataagtctaggtggcagcagggcaacgtgttttcctgttctgtgatgcacgaggccctgcacaatcactacacacagaagagcctgtccctgtctcccggcaagtgagatatc

SEQ ID NO: 39 - protein, 645 aa, synthetic construct
  REGION 1..645: note = designed LILRB2 variants
QTGTIPKPTLWAEPDSVITQGSPVTLSCQGSLEAQEYRLYREKKSASWIFRIRPELVKNGQFHIPSITWEHTGRYGCQYYSRARWSELSDPLVLVMTGAYPKPTLSAQPSPVVTSGGRVTLQCESQVAFGGFILCKEGEEEHPQCLNSQPHARGSSRAIFSVGPVSPNRRWSHRCYGYDLNSPYVWSSPSDLLELLVPGVSKKPSLSVQPGPVVAPGESLTLQCVSDVGYDRFVLYKEGERDLRQLPGRQPQAGLSQANFTLGPVSRSYGGQYRCYGAHNLSSECSAPSDPLDILITGQIRGTPFISVQPGPTVASGENVTLLCQSWRQFHTFLLTKAGAADAPLRLRSIHEYPKYQAEFPMSPVTSAHAGTYRCYGSLNSDPYLLSHPSEPLELVVSGGGGSGGGGSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 40 - DNA, 1944 bp, synthetic construct
  misc_feature 1..1944: note = designed LILRB2 variants
  source 1..1944: mol_type = other DNA
cagaccggcacaatcccaaagcccaccctgtgggccgagcctgattccgtgatcacccagggctctccagtgacactgtcctgccagggctctctggaggcccaggagtaccggctgtatagagagaagaagtctgccagctggatcttccggatcagacctgagctggtgaagaacggccagtttcacatcccaagcatcacctgggagcacacaggccggtacggatgccagtactattcccgggccagatggagcgagctgtccgaccctctggtgctggtcatgaccggcgcctatcctaagccaacactgagcgcccagccatcccctgtggtgaccagcggcggcagagtgacactgcagtgtgagtcccaggtggccttcggcggctttatcctgtgcaaggagggcgaggaggagcacccacagtgtctgaacagccagccacacgcccggggcagctccagagccatcttctccgtgggacccgtgagcccaaaccggagatggagccaccggtgctacggctatgacctgaatagcccttacgtgtggtctagcccatccgatctgctggagctgctggtgcccggcgtgtccaagaagccttccctgtctgtgcagccaggaccagtggtggcaccaggagagtctctgaccctgcagtgcgtgagcgacgtgggctacgatcggttcgtgctgtataaggagggagagagggatctgaggcagctgccaggcagacagccacaggccggcctgagccaggccaactttacactgggcccagtgagcaggtcctatggcggacagtacaggtgctatggagcacacaatctgtcctctgagtgttctgcccccagcgaccccctggacatcctgatcaccggccagatcaggggcacacccttcatctccgtgcagcctggaccaaccgtggcctctggcgagaacgtgacactgctgtgccagtcttggcgccagttccacacctttctgctgacaaaggcaggagcagcagacgcaccactgaggctgcgcagcatccacgagtaccccaagtatcaggccgagtttccaatgtctccagtgaccagcgcccacgcaggcacatacaggtgttatggcagcctgaacagcgacccctacctgctgagccacccttccgagccactggagctggtggtgagcggaggaggaggctccggaggaggaggctctggcggcggcggcagcgagcctaagagctccgacaagacccacacatgcccaccttgtccagcacctgaggcagcaggaggaccatccgtgttcctgtttccacccaagcctaaggataccctgatgatctctcgcacccctgaggtgacatgcgtggtggtggacgtgagccacgaggaccccgaggtgaagtttaactggtacgtggacggcgtggaggtgcacaatgccaagacaaagccccgggaggagcagtacaacagcacctatagagtggtgtccgtgctgacagtgctgcaccaggattggctgaacggcaaggagtacaagtgtaaggtgtccaataaggccctgccagcccccatcgagaagaccatctctaaggcaaagggacagcccagggagcctcaggtgtgcaccctgcctccaagccgcgacgagctgacaaagaaccaggtgtctctgagctgtgccgtgaagggcttctacccatctgacatcgccgtggagtgggagagcaatggccagcccgagaacaattataagaccacaccccctgtgctggactctgatggcagcttctttctggtgtccaagctgaccgtggataagtctaggtggcagcagggcaacgtgttttcctgttctgtgatgcacgaggccctgcacaatcactacacacagaagagcctgtccctgtctcccggcaagtgagatatc

SEQ ID NO: 41 - protein, 645 aa, synthetic construct
  REGION 1..645: note = designed LILRB2 variants
QTGTIPKPTLWAEPDSVITQGSPVTLSCQGSLEAQEYRLYREKKSASWITRIRPELRKNGQFHIPSITWEHTGRYGCQYYSRARWSELSDPLVLVMTGAYPKPTLSAQPSPVVTSGGRVTLQCESQVAFGGFILCKEGEEEHPQCLNSQPHARGSSRAIFSVGPVSPNRRWSHRCYGYDLNSPYVWSSPSDLLELLVPGVSKKPSLSVQPGPVVAPGESLTLQCVSDVGYDRFVLYKEGERDLRQLPGRQPQAGLSQANFTLGPVSRSYGGQYRCYGAHNLSSECSAPSDPLDILITGQIRGTPFISVQPGPTVASGENVTLLCQSWRQFHTFLLTKAGAADAPLRLRSIHEYPKYQAEFPMSPVTSAHAGTYRCYGSLNSDPYLLSHPSEPLELVVSGGGGSGGGGSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 42 - DNA, 1944 bp, synthetic construct
  misc_feature 1..1944: note = designed LILRB2 variants
  source 1..1944: mol_type = other DNA
cagaccggcacaatcccaaagcccaccctgtgggccgagcctgattccgtgatcacccagggctctccagtgacactgtcctgccagggctctctggaggcccaggagtaccggctgtatagagagaagaagtctgccagctggatcacccggatcagacctgagctgcggaagaacggccagtttcacatcccaagcatcacctgggagcacacaggccggtacggatgccagtactattcccgggccagatggagcgagctgtccgaccctctggtgctggtcatgaccggcgcctatcctaagccaacactgagcgcccagccatcccctgtggtgaccagcggcggcagagtgacactgcagtgtgagtcccaggtggccttcggcggctttatcctgtgcaaggagggcgaggaggagcacccacagtgtctgaacagccagccacacgcccggggcagctccagagccatcttctccgtgggacccgtgagcccaaaccggagatggagccaccggtgctacggctatgacctgaatagcccttacgtgtggtctagcccatccgatctgctggagctgctggtgcccggcgtgtccaagaagccttccctgtctgtgcagccaggaccagtggtggcaccaggagagtctctgaccctgcagtgcgtgagcgacgtgggctacgatcggttcgtgctgtataaggagggagagagggatctgaggcagctgccaggcagacagccacaggccggcctgagccaggccaactttacactgggcccagtgagcaggtcctatggcggacagtacaggtgctatggagcacacaatctgtcctctgagtgttctgcccccagcgaccccctggacatcctgatcaccggccagatcaggggcacacccttcatctccgtgcagcctggaccaaccgtggcctctggcgagaacgtgacactgctgtgccagtcttggcgccagttccacacctttctgctgacaaaggcaggagcagcagacgcaccactgaggctgcgcagcatccacgagtaccccaagtatcaggccgagtttccaatgtctccagtgaccagcgcccacgcaggcacatacaggtgttatggcagcctgaacagcgacccctacctgctgagccacccttccgagccactggagctggtggtgagcggaggaggaggctccggaggaggaggctctggcggcggcggcagcgagcctaagagctccgacaagacccacacatgcccaccttgtccagcacctgaggcagcaggaggaccatccgtgttcctgtttccacccaagcctaaggataccctgatgatctctcgcacccctgaggtgacatgcgtggtggtggacgtgagccacgaggaccccgaggtgaagtttaactggtacgtggacggcgtggaggtgcacaatgccaagacaaagccccgggaggagcagtacaacagcacctatagagtggtgtccgtgctgacagtgctgcaccaggattggctgaacggcaaggagtacaagtgtaaggtgtccaataaggccctgccagcccccatcgagaagaccatctctaaggcaaagggacagcccagggagcctcaggtgtgcaccctgcctccaagccgcgacgagctgacaaagaaccaggtgtctctgagctgtgccgtgaagggcttctacccatctgacatcgccgtggagtgggagagcaatggccagcccgagaacaattataagaccacaccccctgtgctggactctgatggcagcttctttctggtgtccaagctgaccgtggataagtctaggtggcagcagggcaacgtgttttcctgttctgtgatgcacgaggccctgcacaatcactacacacagaagagcctgtccctgtctcccggcaagtgagatatc

SEQ ID NO: 43 - protein, 645 aa, synthetic construct
  REGION 1..645: note = designed LILRB2 variants
QTGTIPKPTLWAEPDSVITQGSPVTLSCQGSLEAQEYRLYREKKQASWITRIRPELVKNGQFHIPSITWEHTGRYGCQYYSRARWSELSDPLVLVMTGAYPKPTLSAQPSPVVTSGGRVTLQCESQVAFGGFILCKEGEEEHPQCLNSQPHARGSSRAIFSVGPVSPNRRWSHRCYGYDLNSPYVWSSPSDLLELLVPGVSKKPSLSVQPGPVVAPGESLTLQCVSDVGYDRFVLYKEGERDLRQLPGRQPQAGLSQANFTLGPVSRSYGGQYRCYGAHNLSSECSAPSDPLDILITGQIRGTPFISVQPGPTVASGENVTLLCQSWRQFHTFLLTKAGAADAPLRLRSIHEYPKYQAEFPMSPVTSAHAGTYRCYGSLNSDPYLLSHPSEPLELVVSGGGGSGGGGSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 44 - DNA, 1944 bp, synthetic construct
  misc_feature 1..1944: note = designed LILRB2 variants
  source 1..1944: mol_type = other DNA
cagaccggcacaatcccaaagcccaccctgtgggccgagcctgattccgtgatcacccagggctctccagtgacactgtcctgccagggctctctggaggcccaggagtaccggctgtatagagagaagaagcaggccagctggatcacccggatcagacctgagctggtgaagaacggccagtttcacatcccaagcatcacctgggagcacacaggccggtacggatgccagtactattcccgggccagatggagcgagctgtccgaccctctggtgctggtcatgaccggcgcctatcctaagccaacactgagcgcccagccatcccctgtggtgaccagcggcggcagagtgacactgcagtgtgagtcccaggtggccttcggcggctttatcctgtgcaaggagggcgaggaggagcacccacagtgtctgaacagccagccacacgcccggggcagctccagagccatcttctccgtgggacccgtgagcccaaaccggagatggagccaccggtgctacggctatgacctgaatagcccttacgtgtggtctagcccatccgatctgctggagctgctggtgcccggcgtgtccaagaagccttccctgtctgtgcagccaggaccagtggtggcaccaggagagtctctgaccctgcagtgcgtgagcgacgtgggctacgatcggttcgtgctgtataaggagggagagagggatctgaggcagctgccaggcagacagccacaggccggcctgagccaggccaactttacactgggcccagtgagcaggtcctatggcggacagtacaggtgctatggagcacacaatctgtcctctgagtgttctgcccccagcgaccccctggacatcctgatcaccggccagatcaggggcacacccttcatctccgtgcagcctggaccaaccgtggcctctggcgagaacgtgacactgctgtgccagtcttggcgccagttccacacctttctgctgacaaaggcaggagcagcagacgcaccactgaggctgcgcagcatccacgagtaccccaagtatcaggccgagtttccaatgtctccagtgaccagcgcccacgcaggcacatacaggtgttatggcagcctgaacagcgacccctacctgctgagccacccttccgagccactggagctggtggtgagcggaggaggaggctccggaggaggaggctctggcggcggcggcagcgagcctaagagctccgacaagacccacacatgcccaccttgtccagcacctgaggcagcaggaggaccatccgtgttcctgtttccacccaagcctaaggataccctgatgatctctcgcacccctgaggtgacatgcgtggtggtggacgtgagccacgaggaccccgaggtgaagtttaactggtacgtggacggcgtggaggtgcacaatgccaagacaaagccccgggaggagcagtacaacagcacctatagagtggtgtccgtgctgacagtgctgcaccaggattggctgaacggcaaggagtacaagtgtaaggtgtccaataaggccctgccagcccccatcgagaagaccatctctaaggcaaagggacagcccagggagcctcaggtgtgcaccctgcctccaagccgcgacgagctgacaaagaaccaggtgtctctgagctgtgccgtgaagggcttctacccatctgacatcgccgtggagtgggagagcaatggccagcccgagaacaattataagaccacaccccctgtgctggactctgatggcagcttctttctggtgtccaagctgaccgtggataagtctaggtggcagcagggcaacgtgttttcctgttctgtgatgcacgaggccctgcacaatcactacacacagaagagcctgtccctgtctcccggcaagtgagatatc

SEQ ID NO: 45 - protein, 645 aa, synthetic construct
  REGION 1..645: note = designed LILRB2 variants
QTGTIPKPTLWAEPDSVITQGSPVTLSCQGSLEAQEYRLYREKKSASWKTRIRPELVKNGQFHIPSITWEHTGRYGCQYYSRARWSELSDPLVLVMTGAYPKPTLSAQPSPVVTSGGRVTLQCESQVAFGGFILCKEGEEEHPQCLNSQPHARGSSRAIFSVGPVSPNRRWSHRCYGYDLNSPYVWSSPSDLLELLVPGVSKKPSLSVQPGPVVAPGESLTLQCVSDVGYDRFVLYKEGERDLRQLPGRQPQAGLSQANFTLGPVSRSYGGQYRCYGAHNLSSECSAPSDPLDILITGQIRGTPFISVQPGPTVASGENVTLLCQSWRQFHTFLLTKAGAADAPLRLRSIHEYPKYQAEFPMSPVTSAHAGTYRCYGSLNSDPYLLSHPSEPLELVVSGGGGSGGGGSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 46 - DNA, 1944 bp, synthetic construct
  misc_feature 1..1944: note = designed LILRB2 variants
  source 1..1944: mol_type = other DNA
cagaccggcacaatcccaaagcccaccctgtgggccgagcctgattccgtgatcacccagggctctccagtgacactgtcctgccagggctctctggaggcccaggagtaccggctgtatagagagaagaagtctgccagctggaagacccggatcagacctgagctggtgaagaacggccagtttcacatcccaagcatcacctgggagcacacaggccggtacggatgccagtactattcccgggccagatggagcgagctgtccgaccctctggtgctggtcatgaccggcgcctatcctaagccaacactgagcgcccagccatcccctgtggtgaccagcggcggcagagtgacactgcagtgtgagtcccaggtggccttcggcggctttatcctgtgcaaggagggcgaggaggagcacccacagtgtctgaacagccagccacacgcccggggcagctccagagccatcttctccgtgggacccgtgagcccaaaccggagatggagccaccggtgctacggctatgacctgaatagcccttacgtgtggtctagcccatccgatctgctggagctgctggtgcccggcgtgtccaagaagccttccctgtctgtgcagccaggaccagtggtggcaccaggagagtctctgaccctgcagtgcgtgagcgacgtgggctacgatcggttcgtgctgtataaggagggagagagggatctgaggcagctgccaggcagacagccacaggccggcctgagccaggccaactttacactgggcccagtgagcaggtcctatggcggacagtacaggtgctatggagcacacaatctgtcctctgagtgttctgcccccagcgaccccctggacatcctgatcaccggccagatcaggggcacacccttcatctccgtgcagcctggaccaaccgtggcctctggcgagaacgtgacactgctgtgccagtcttggcgccagttccacacctttctgctgacaaaggcaggagcagcagacgcaccactgaggctgcgcagcatccacgagtaccccaagtatcaggccgagtttccaatgtctccagtgaccagcgcccacgcaggcacatacaggtgttatggcagcctgaacagcgacccctacctgctgagccacccttccgagccactggagctggtggtgagcggaggaggaggctccggaggaggaggctctggcggcggcggcagcgagcctaagagctccgacaagacccacacatgcccaccttgtccagcacctgaggcagcaggaggaccatccgtgttcctgtttccacccaagcctaaggataccctgatgatctctcgcacccctgaggtgacatgcgtggtggtggacgtgagccacgaggaccccgaggtgaagtttaactggtacgtggacggcgtggaggtgcacaatgccaagacaaagccccgggaggagcagtacaacagcacctatagagtggtgtccgtgctgacagtgctgcaccaggattggctgaacggcaaggagtacaagtgtaaggtgtccaataaggccctgccagcccccatcgagaagaccatctctaaggcaaagggacagcccagggagcctcaggtgtgcaccctgcctccaagccgcgacgagctgacaaagaaccaggtgtctctgagctgtgccgtgaagggcttctacccatctgacatcgccgtggagtgggagagcaatggccagcccgagaacaattataagaccacaccccctgtgctggactctgatggcagcttctttctggtgtccaagctgaccgtggataagtctaggtggcagcagggcaacgtgttttcctgttctgtgatgcacgaggccctgcacaatcactacacacagaagagcctgtccctgtctcccggcaagtgagatatc

SEQ ID NO: 47 - protein, 645 aa, synthetic construct
  REGION 1..645: note = designed LILRB2 variants
QTGTIPKPTLWAEPDSVITQGSPVTLSCQGSLEAQEYRLYREKKQASWKTRIRPELVKNGQFHIPSITWEHTGRYGCQYYSRARWSELSDPLVLVMTGAYPKPTLSAQPSPVVTSGGRVTLQCESQVAFGGFILCKEGEEEHPQCLNSQPHARGSSRAIFSVGPVSPNRRWSHRCYGYDLNSPYVWSSPSDLLELLVPGVSKKPSLSVQPGPVVAPGESLTLQCVSDVGYDRFVLYKEGERDLRQLPGRQPQAGLSQANFTLGPVSRSYGGQYRCYGAHNLSSECSAPSDPLDILITGQIRGTPFISVQPGPTVASGENVTLLCQSWRQFHTFLLTKAGAADAPLRLRSIHEYPKYQAEFPMSPVTSAHAGTYRCYGSLNSDPYLLSHPSEPLELVVSGGGGSGGGGSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 48 - DNA, 1944 bp, synthetic construct
  misc_feature 1..1944: note = designed LILRB2 variants
  source 1..1944: mol_type = other DNA
cagaccggcacaatcccaaagcccaccctgtgggccgagcctgattccgtgatcacccagggctctccagtgacactgtcctgccagggctctctggaggcccaggagtaccggctgtatagagagaagaagcaggccagctggaagacccggatcagacctgagctggtgaagaacggccagtttcacatcccaagcatcacctgggagcacacaggccggtacggatgccagtactattcccgggccagatggagcgagctgtccgaccctctggtgctggtcatgaccggcgcctatcctaagccaacactgagcgcccagccatcccctgtggtgaccagcggcggcagagtgacactgcagtgtgagtcccaggtggccttcggcggctttatcctgtgcaaggagggcgaggaggagcacccacagtgtctgaacagccagccacacgcccggggcagctccagagccatcttctccgtgggacccgtgagcccaaaccggagatggagccaccggtgctacggctatgacctgaatagcccttacgtgtggtctagcccatccgatctgctggagctgctggtgcccggcgtgtccaagaagccttccctgtctgtgcagccaggaccagtggtggcaccaggagagtctctgaccctgcagtgcgtgagcgacgtgggctacgatcggttcgtgctgtataaggagggagagagggatctgaggcagctgccaggcagacagccacaggccggcctgagccaggccaactttacactgggcccagtgagcaggtcctatggcggacagtacaggtgctatggagcacacaatctgtcctctgagtgttctgcccccagcgaccccctggacatcctgatcaccggccagatcaggggcacacccttcatctccgtgcagcctggaccaaccgtggcctctggcgagaacgtgacactgctgtgccagtcttggcgccagttccacacctttctgctgacaaaggcaggagcagcagacgcaccactgaggctgcgcagcatccacgagtaccccaagtatcaggccgagtttccaatgtctccagtgaccagcgcccacgcaggcacatacaggtgttatggcagcctgaacagcgacccctacctgctgagccacccttccgagccactggagctggtggtgagcggaggaggaggctccggaggaggaggctctggcggcggcggcagcgagcctaagagctccgacaagacccacacatgcccaccttgtccagcacctgaggcagcaggaggaccatccgtgttcctgtttccacccaagcctaaggataccctgatgatctctcgcacccctgaggtgacatgcgtggtggtggacgtgagccacgaggaccccgaggtgaagtttaactggtacgtggacggcgtggaggtgcacaatgccaagacaaagccccgggaggagcagtacaacagcacctatagagtggtgtccgtgctgacagtgctgcaccaggattggctgaacggcaaggagtacaagtgtaaggtgtccaataaggccctgccagcccccatcgagaagaccatctctaaggcaaagggacagcccagggagcctcaggtgtgcaccctgcctccaagccgcgacgagctgacaaagaaccaggtgtctctgagctgtgccgtgaagggcttctacccatctgacatcgccgtggagtgggagagcaatggccagcccgagaacaattataagaccacaccccctgtgctggactctgatggcagcttctttctggtgtccaagctgaccgtggataagtctaggtggcagcagggcaacgtgttttcctgttctgtgatgcacgaggccctgcacaatcactacacacagaagagcctgtccctgtctcccggcaagtgagatatc

SEQ ID NO: 49 - protein, 645 aa, synthetic construct
  REGION 1..645: note = designed LILRB2 variants
QTGTIPKPTLWAEPDSVITQGSPVTLSCQGSLEAQEYRLYREKKQASWITRIRPELRKNGQFHIPSITWEHTGRYGCQYYSRARWSELSDPLVLVMTGAYPKPTLSAQPSPVVTSGGRVTLQCESQVAFGGFILCKEGEEEHPQCLNSQPHARGSSRAIFSVGPVSPNRRWSHRCYGYDLNSPYVWSSPSDLLELLVPGVSKKPSLSVQPGPVVAPGESLTLQCVSDVGYDRFVLYKEGERDLRQLPGRQPQAGLSQANFTLGPVSRSYGGQYRCYGAHNLSSECSAPSDPLDILITGQIRGTPFISVQPGPTVASGENVTLLCQSWRQFHTFLLTKAGAADAPLRLRSIHEYPKYQAEFPMSPVTSAHAGTYRCYGSLNSDPYLLSHPSEPLELVVSGGGGSGGGGSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 50 - DNA, 1944 bp, synthetic construct
  misc_feature 1..1944: note = designed LILRB2 variants
  source 1..1944: mol_type = other DNA
cagaccggcacaatcccaaagcccaccctgtgggccgagcctgattccgtgatcacccagggctctccagtgacactgtcctgccagggctctctggaggcccaggagtaccggctgtatagagagaagaagcaggccagctggatcacccggatcagacctgagctgagaaagaacggccagtttcacatcccaagcatcacctgggagcacacaggccggtacggatgccagtactattcccgggccagatggagcgagctgtccgaccctctggtgctggtcatgaccggcgcctatcctaagccaacactgagcgcccagccatcccctgtggtgaccagcggcggcagagtgacactgcagtgtgagtcccaggtggccttcggcggctttatcctgtgcaaggagggcgaggaggagcacccacagtgtctgaacagccagccacacgcccggggcagctccagagccatcttctccgtgggacccgtgagcccaaaccggagatggagccaccggtgctacggctatgacctgaatagcccttacgtgtggtctagcccatccgatctgctggagctgctggtgcccggcgtgtccaagaagccttccctgtctgtgcagccaggaccagtggtggcaccaggagagtctctgaccctgcagtgcgtgagcgacgtgggctacgatcggttcgtgctgtataaggagggagagagggatctgaggcagctgccaggcagacagccacaggccggcctgagccaggccaactttacactgggcccagtgagcaggtcctatggcggacagtacaggtgctatggagcacacaatctgtcctctgagtgttctgcccccagcgaccccctggacatcctgatcaccggccagatcaggggcacacccttcatctccgtgcagcctggaccaaccgtggcctctggcgagaacgtgacactgctgtgccagtcttggcgccagttccacacctttctgctgacaaaggcaggagcagcagacgcaccactgaggctgcgcagcatccacgagtaccccaagtatcaggccgagtttccaatgtctccagtgaccagcgcccacgcaggcacatacaggtgttatggcagcctgaacagcgacccctacctgctgagccacccttccgagccactggagctggtggtgagcggaggaggaggctccggaggaggaggctctggcggcggcggcagcgagcctaagagctccgacaagacccacacatgcccaccttgtccagcacctgaggcagcaggaggaccatccgtgttcctgtttccacccaagcctaaggataccctgatgatctctcgcacccctgaggtgacatgcgtggtggtggacgtgagccacgaggaccccgaggtgaagtttaactggtacgtggacggcgtggaggtgcacaatgccaagacaaagccccgggaggagcagtacaacagcacctatagagtggtgtccgtgctgacagtgctgcaccaggattggctgaacggcaaggagtacaagtgtaaggtgtccaataaggccctgccagcccccatcgagaagaccatctctaaggcaaagggacagcccagggagcctcaggtgtgcaccctgcctccaagccgcgacgagctgacaaagaaccaggtgtctctgagctgtgccgtgaagggcttctacccatctgacatcgccgtggagtgggagagcaatggccagcccgagaacaattataagaccacaccccctgtgctggactctgatggcagcttctttctggtgtccaagctgaccgtggataagtctaggtggcagcagggcaacgtgttttcctgttctgtgatgcacgaggccctgcacaatcactacacacagaagagcctgtccctgtctcccggcaagtgagatatc

SEQ ID NO: 51 - protein, 645 aa, synthetic construct
  REGION 1..645: note = designed LILRB2 variants
QTGTIPKPTLWAEPDSVITQGSPVTLSCQGSLEAQEYRLYREKKRASWFTRIRPELVKNGQFHIPSITWEHTGRYGCQYYSRARWSELSDPLVLVMTGAYPKPTLSAQPSPVVTSGGRVTLQCESQVAFGGFILCKEGEEEHPQCLNSQPHARGSSRAIFSVGPVSPNRRWSHRCYGYDLNSPYVWSSPSDLLELLVPGVSKKPSLSVQPGPVVAPGESLTLQCVSDVGYDRFVLYKEGERDLRQLPGRQPQAGLSQANFTLGPVSRSYGGQYRCYGAHNLSSECSAPSDPLDILITGQIRGTPFISVQPGPTVASGENVTLLCQSWRQFHTFLLTKAGAADAPLRLRSIHEYPKYQAEFPMSPVTSAHAGTYRCYGSLNSDPYLLSHPSEPLELVVSGGGGSGGGGSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 52 - DNA, 1944 bp, synthetic construct
  misc_feature 1..1944: note = designed LILRB2 variants
  source 1..1944: mol_type = other DNA
cagaccggcacaatcccaaagcccaccctgtgggccgagcctgattccgtgatcacccagggctctccagtgacactgtcctgccagggctctctggaggcccaggagtaccggctgtatagagagaagaagagagccagctggttcacccggatcagacctgagctggtgaagaacggccagtttcacatcccaagcatcacctgggagcacacaggccggtacggatgccagtactattcccgggccagatggagcgagctgtccgaccctctggtgctggtcatgaccggcgcctatcctaagccaacactgagcgcccagccatcccctgtggtgaccagcggcggcagagtgacactgcagtgtgagtcccaggtggccttcggcggctttatcctgtgcaaggagggcgaggaggagcacccacagtgtctgaacagccagccacacgcccggggcagctccagagccatcttctccgtgggacccgtgagcccaaaccggagatggagccaccggtgctacggctatgacctgaatagcccttacgtgtggtctagcccatccgatctgctggagctgctggtgcccggcgtgtccaagaagccttccctgtctgtgcagccaggaccagtggtggcaccaggagagtctctgaccctgcagtgcgtgagcgacgtgggctacgatcggttcgtgctgtataaggagggagagagggatctgaggcagctgccaggcagacagccacaggccggcctgagccaggccaactttacactgggcccagtgagcaggtcctatggcggacagtacaggtgctatggagcacacaatctgtcctctgagtgttctgcccccagcgaccccctggacatcctgatcaccggccagatcaggggcacacccttcatctccgtgcagcctggaccaaccgtggcctctggcgagaacgtgacactgctgtgccagtcttggcgccagttccacacctttctgctgacaaaggcaggagcagcagacgcaccactgaggctgcgcagcatccacgagtaccccaagtatcaggccgagtttccaatgtctccagtgaccagcgcccacgcaggcacatacaggtgttatggcagcctgaacagcgacccctacctgctgagccacccttccgagccactggagctggtggtgagcggaggaggaggctccggaggaggaggctctggcggcggcggcagcgagcctaagagctccgacaagacccacacatgcccaccttgtccagcacctgaggcagcaggaggaccatccgtgttcctgtttccacccaagcctaaggataccctgatgatctctcgcacccctgaggtgacatgcgtggtggtggacgtgagccacgaggaccccgaggtgaagtttaactggtacgtggacggcgtggaggtgcacaatgccaagacaaagccccgggaggagcagtacaacagcacctatagagtggtgtccgtgctgacagtgctgcaccaggattggctgaacggcaaggagtacaagtgtaaggtgtccaataaggccctgccagcccccatcgagaagaccatctctaaggcaaagggacagcccagggagcctcaggtgtgcaccctgcctccaagccgcgacgagctgacaaagaaccaggtgtctctgagctgtgccgtgaagggcttctacccatctgacatcgccgtggagtgggagagcaatggccagcccgagaacaattataagaccacaccccctgtgctggactctgatggcagcttctttctggtgtccaagctgaccgtggataagtctaggtggcagcagggcaacgtgttttcctgttctgtgatgcacgaggccctgcacaatcactacacacagaagagcctgtccctgtctcccggcaagtgagatatc

SEQ ID NO: 53 - protein, 645 aa, synthetic construct
  REGION 1..645: note = designed LILRB2 variants
QTGTIPKPTLWAEPDSVITQGSPVTLSCQGSLEAQEYRLYREKKYASWIKRIRPELVKNGQFHIPSITWEHTGRYGCQYYSRARWSELSDPLVLVMTGAYPKPTLSAQPSPVVTSGGRVTLQCESQVAFGGFILCKEGEEEHPQCLNSQPHARGSSRAIFSVGPVSPNRRWSHRCYGYDLNSPYVWSSPSDLLELLVPGVSKKPSLSVQPGPVVAPGESLTLQCVSDVGYDRFVLYKEGERDLRQLPGRQPQAGLSQANFTLGPVSRSYGGQYRCYGAHNLSSECSAPSDPLDILITGQIRGTPFISVQPGPTVASGENVTLLCQSWRQFHTFLLTKAGAADAPLRLRSIHEYPKYQAEFPMSPVTSAHAGTYRCYGSLNSDPYLLSHPSEPLELVVSGGGGSGGGGSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 54 - DNA, 1944 bp, synthetic construct
  misc_feature 1..1944: note = designed LILRB2 variants
  source 1..1944: mol_type = other DNA
cagaccggcacaatcccaaagcccaccctgtgggccgagcctgattccgtgatcacccagggctctccagtgacactgtcctgccagggctctctggaggcccaggagtaccggctgtatagagagaagaagtacgccagctggatcaagcggatcagacctgagctggtgaagaacggccagtttcacatcccaagcatcacctgggagcacacaggccggtacggatgccagtactattcccgggccagatggagcgagctgtccgaccctctggtgctggtcatgaccggcgcctatcctaagccaacactgagcgcccagccatcccctgtggtgaccagcggcggcagagtgacactgcagtgtgagtcccaggtggccttcggcggctttatcctgtgcaaggagggcgaggaggagcacccacagtgtctgaacagccagccacacgcccggggcagctccagagccatcttctccgtgggacccgtgagcccaaaccggagatggagccaccggtgctacggctatgacctgaatagcccttacgtgtggtctagcccatccgatctgctggagctgctggtgcccggcgtgtccaagaagccttccctgtctgtgcagccaggaccagtggtggcaccaggagagtctctgaccctgcagtgcgtgagcgacgtgggctacgatcggttcgtgctgtataaggagggagagagggatctgaggcagctgccaggcagacagccacaggccggcctgagccaggccaactttacactgggcccagtgagcaggtcctatggcggacagtacaggtgctatggagcacacaatctgtcctctgagtgttctgcccccagcgaccccctggacatcctgatcaccggccagatcaggggcacacccttcatctccgtgcagcctggaccaaccgtggcctctggcgagaacgtgacactgctgtgccagtcttggcgccagttccacacctttctgctgacaaaggcaggagcagcagacgcaccactgaggctgcgcagcatccacgagtaccccaagtatcaggccgagtttccaatgtctccagtgaccagcgcccacgcaggcacatacaggtgttatggcagcctgaacagcgacccctacctgctgagccacccttccgagccactggagctggtggtgagcggaggaggaggctccggaggaggaggctctggcggcggcggcagcgagcctaagagctccgacaagacccacacatgcccaccttgtccagcacctgaggcagcaggaggaccatccgtgttcctgtttccacccaagcctaaggataccctgatgatctctcgcacccctgaggtgacatgcgtggtggtggacgtgagccacgaggaccccgaggtgaagtttaactggtacgtggacggcgtggaggtgcacaatgccaagacaaagccccgggaggagcagtacaacagcacctatagagtggtgtccgtgctgacagtgctgcaccaggattggctgaacggcaaggagtacaagtgtaaggtgtccaataaggccctgccagcccccatcgagaagaccatctctaaggcaaagggacagcccagggagcctcaggtgtgcaccctgcctccaagccgcgacgagctgacaaagaaccaggtgtctctgagctgtgccgtgaagggcttctacccatctgacatcgccgtggagtgggagagcaatggccagcccgagaacaattataagaccacaccccctgtgctggactctgatggcagcttctttctggtgtccaagctgaccgtggataagtctaggtggcagcagggcaacgtgttttcctgttctgtgatgcacgaggccctgcacaatcactacacacagaagagcctgtccctgtctcccggcaagtgagatatc

SEQ ID NO: 55 - protein, 645 aa, synthetic construct
  REGION 1..645: note = designed LILRB2 variants
QTGTIPKPTLWAEPDSVITQGSPVTLSCQGSLEAQEYRLYREKKYASWINRIRPELVKNGQFHIPSITWEHTGRYGCQYYSRARWSELSDPLVLVMTGAYPKPTLSAQPSPVVTSGGRVTLQCESQVAFGGFILCKEGEEEHPQCLNSQPHARGSSRAIFSVGPVSPNRRWSHRCYGYDLNSPYVWSSPSDLLELLVPGVSKKPSLSVQPGPVVAPGESLTLQCVSDVGYDRFVLYKEGERDLRQLPGRQPQAGLSQANFTLGPVSRSYGGQYRCYGAHNLSSECSAPSDPLDILITGQIRGTPFISVQPGPTVASGENVTLLCQSWRQFHTFLLTKAGAADAPLRLRSIHEYPKYQAEFPMSPVTSAHAGTYRCYGSLNSDPYLLSHPSEPLELVVSGGGGSGGGGSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 56 - DNA, 1944 bp, synthetic construct
  misc_feature 1..1944: note = designed LILRB2 variants
  source 1..1944: mol_type = other DNA
cagaccggcacaatcccaaagcccaccctgtgggccgagcctgattccgtgatcacccagggctctccagtgacactgtcctgccagggctctctggaggcccaggagtaccggctgtatagagagaagaagtacgccagctggatcaaccggatcagacctgagctggtgaagaacggccagtttcacatcccaagcatcacctgggagcacacaggccggtacggatgccagtactattcccgggccagatggagcgagctgtccgaccctctggtgctggtcatgaccggcgcctatcctaagccaacactgagcgcccagccatcccctgtggtgaccagcggcggcagagtgacactgcagtgtgagtcccaggtggccttcggcggctttatcctgtgcaaggagggcgaggaggagcacccacagtgtctgaacagccagccacacgcccggggcagctccagagccatcttctccgtgggacccgtgagcccaaaccggagatggagccaccggtgctacggctatgacctgaatagcccttacgtgtggtctagcccatccgatctgctggagctgctggtgcccggcgtgtccaagaagccttccctgtctgtgcagccaggaccagtggtggcaccaggagagtctctgaccctgcagtgcgtgagcgacgtgggctacgatcggttcgtgctgtataaggagggagagagggatctgaggcagctgccaggcagacagccacaggccggcctgagccaggccaactttacactgggcccagtgagcaggtcctatggcggacagtacaggtgctatggagcacacaatctgtcctctgagtgttctgcccccagcgaccccctggacatcctgatcaccggccagatcaggggcacacccttcatctccgtgcagcctggaccaaccgtggcctctggcgagaacgtgacactgctgtgccagtcttggcgccagttccacacctttctgctgacaaaggcaggagcagcagacgcaccactgaggctgcgcagcatccacgagtaccccaagtatcaggccgagtttccaatgtctccagtgaccagcgcccacgcaggcacatacaggtgttatggcagcctgaacagcgacccctacctgctgagccacccttccgagccactggagctggtggtgagcggaggaggaggctccggaggaggaggctctggcggcggcggcagcgagcctaagagctccgacaagacccacacatgcccaccttgtccagcacctgaggcagcaggaggaccatccgtgttcctgtttccacccaagcctaaggataccctgatgatctctcgcacccctgaggtgacatgcgtggtggtggacgtgagccacgaggaccccgaggtgaagtttaactggtacgtggacggcgtggaggtgcacaatgccaagacaaagccccgggaggagcagtacaacagcacctatagagtggtgtccgtgctgacagtgctgcaccaggattggctgaacggcaaggagtacaagtgtaaggtgtccaataaggccctgccagcccccatcgagaagaccatctctaaggcaaagggacagcccagggagcctcaggtgtgcaccctgcctccaagccgcgacgagctgacaaagaaccaggtgtctctgagctgtgccgtgaagggcttctacccatctgacatcgccgtggagtgggagagcaatggccagcccgagaacaattataagaccacaccccctgtgctggactctgatggcagcttctttctggtgtccaagctgaccgtggataagtctaggtggcagcagggcaacgtgttttcctgttctgtgatgcacgaggccctgcacaatcactacacacagaagagcctgtccctgtctcccggcaagtgagatatc

SEQ ID NO: 57 - protein, 645 aa, synthetic construct
  REGION 1..645: note = designed LILRB2 variants
QTGTIPKPTLWAEPDSVITQGSPVTLSCQGSLEAQEYRLYREKKNASWIRRIRPELVKNGQFHIPSITWEHTGRYGCQYYSRARWSELSDPLVLVMTGAYPKPTLSAQPSPVVTSGGRVTLQCESQVAFGGFILCKEGEEEHPQCLNSQPHARGSSRAIFSVGPVSPNRRWSHRCYGYDLNSPYVWSSPSDLLELLVPGVSKKPSLSVQPGPVVAPGESLTLQCVSDVGYDRFVLYKEGERDLRQLPGRQPQAGLSQANFTLGPVSRSYGGQYRCYGAHNLSSECSAPSDPLDILITGQIRGTPFISVQPGPTVASGENVTLLCQSWRQFHTFLLTKAGAADAPLRLRSIHEYPKYQAEFPMSPVTSAHAGTYRCYGSLNSDPYLLSHPSEPLELVVSGGGGSGGGGSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 58 - DNA, 1944 bp, synthetic construct
  misc_feature 1..1944: note = designed LILRB2 variants
  source 1..1944: mol_type = other DNA
cagaccggcacaatcccaaagcccaccctgtgggccgagcctgattccgtgatcacccagggctctccagtgacactgtcctgccagggctctctggaggcccaggagtaccggctgtatagagagaagaagaacgccagctggatcagacggatcagacctgagctggtgaagaacggccagtttcacatcccaagcatcacctgggagcacacaggccggtacggatgccagtactattcccgggccagatggagcgagctgtccgaccctctggtgctggtcatgaccggcgcctatcctaagccaacactgagcgcccagccatcccctgtggtgaccagcggcggcagagtgacactgcagtgtgagtcccaggtggccttcggcggctttatcctgtgcaaggagggcgaggaggagcacccacagtgtctgaacagccagccacacgcccggggcagctccagagccatcttctccgtgggacccgtgagcccaaaccggagatggagccaccggtgctacggctatgacctgaatagcccttacgtgtggtctagcccatccgatctgctggagctgctggtgcccggcgtgtccaagaagccttccctgtctgtgcagccaggaccagtggtggcaccaggagagtctctgaccctgcagtgcgtgagcgacgtgggctacgatcggttcgtgctgtataaggagggagagagggatctgaggcagctgccaggcagacagccacaggccggcctgagccaggccaactttacactgggcccagtgagcaggtcctatggcggacagtacaggtgctatggagcacacaatctgtcctctgagtgttctgcccccagcgaccccctggacatcctgatcaccggccagatcaggggcacacccttcatctccgtgcagcctggaccaaccgtggcctctggcgagaacgtgacactgctgtgccagtcttggcgccagttccacacctttctgctgacaaaggcaggagcagcagacgcaccactgaggctgcgcagcatccacgagtaccccaagtatcaggccgagtttccaatgtctccagtgaccagcgcccacgcaggcacatacaggtgttatggcagcctgaacagcgacccctacctgctgagccacccttccgagccactggagctggtggtgagcggaggaggaggctccggaggaggaggctctggcggcggcggcagcgagcctaagagctccgacaagacccacacatgcccaccttgtccagcacctgaggcagcaggaggaccatccgtgttcctgtttccacccaagcctaaggataccctgatgatctctcgcacccctgaggtgacatgcgtggtggtggacgtgagccacgaggaccccgaggtgaagtttaactggtacgtggacggcgtggaggtgcacaatgccaagacaaagccccgggaggagcagtacaacagcacctatagagtggtgtccgtgctgacagtgctgcaccaggattggctgaacggcaaggagtacaagtgtaaggtgtccaataaggccctgccagcccccatcgagaagaccatctctaaggcaaagggacagcccagggagcctcaggtgtgcaccctgcctccaagccgcgacgagctgacaaagaaccaggtgtctctgagctgtgccgtgaagggcttctacccatctgacatcgccgtggagtgggagagcaatggccagcccgagaacaattataagaccacaccccctgtgctggactctgatggcagcttctttctggtgtccaagctgaccgtggataagtctaggtggcagcagggcaacgtgttttcctgttctgtgatgcacgaggccctgcacaatcactacacacagaagagcctgtccctgtctcccggcaagtgagatatc

SEQ ID NO: 59 - protein, 229 aa, synthetic construct
  REGION 1..229: note = hIgG4-SPLE- Fc knob
ESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLWCLVKGFYPSDIAVEWESNGQPENNYKTTPPVLDSDGSFFLYSRLTVDKSRWQEGNVFSCSVMHEALHNHYTQKSLSLSLGK

SEQ ID NO: 60 - protein, 229 aa, synthetic construct
  REGION 1..229: note = Fc (AA 99-327) IgG4 UniProtKB - P01861 S228P(108) L235E (115)plus "Knob" substitutions T366W (246) and Y349C ( for S-Sstabilization).
ESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVCTLPPSQEEMTKNQVSLWCLVKGFYPSDIAVEWESNGQPENNYKTTPPVLDSDGSFFLYSRLTVDKSRWQEGNVFSCSVMHEALHNHYTQKSLSLSLGK

SEQ ID NO: 61 - protein, 229 aa, synthetic construct
  REGION 1..229: note = hIgG4-SPLE- Fc hole
ESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQCEMTKNQVSLSCAVKGFYPSDIAVEWESNGQPENNYKTTPPVLDSDGSFFLVSRLTVDKSRWQEGNVFSCSVMHEALHNHYTQKSLSLSLGK

SEQ ID NO: 62 - protein, 229 aa, synthetic construct; identical to SEQ ID NO: 61
  REGION 1..229: note = Fc (AA 99-327) IgG4 UniProtKB - P01861 S228P (108) L235E (115)plus "Hole" substitutions T366S (246), L368A (248) and Y407V(287), E356C (for S-S stabilization),

SEQ ID NO: 63 - protein, 229 aa, synthetic construct
  REGION 1..229: note = hIgG4 Fc
ESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 64 - protein, 232 aa, synthetic construct
  REGION 1..232: note = hIgG1 Fc
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 65 - protein, 229 aa, synthetic construct
  REGION 1..229: note = hIgG4 Fc knob
ESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVCTLPPSQEEMTKNQVSLYCLVKGFYPSDIAVEWESNGQPENNYKTTPPVLDSDGSFFLYSRLTVDKSRWQEGNVFSCSVMHEALHNHYTQKSLSLSLGK

SEQ ID NO: 66 - protein, 229 aa, synthetic construct
  REGION 1..229: note = hIgG4 Fc hole
ESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSLGK

SEQ ID NO: 67 - protein, 232 aa, synthetic construct
  REGION 1..232: note = hIgG1 Fc knob
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 68 - protein, 232 aa, synthetic construct
  REGION 1..232: note = hIgG1 Fc hole
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLYSKLTVDKSRWQQGNVFSCSVMHEALHNHYTQKSLSLSPGK

SEQ ID NO: 69 - protein, 5 aa, synthetic construct
  REGION 1..5: note = linker amino acid sequence
GGGGS

SEQ ID NO: 70 - protein, 20 aa, synthetic construct
  REGION 1..20: note = linker amino acid sequence
  REPEAT 6..10: note = may be absent
  REPEAT 11..15: note = may be absent
  REPEAT 16..20: note = may be absent
GGGGSGGGGSGGGGSGGGGS

SEQ ID NO: 71 - protein, 9 aa, synthetic construct
  REGION 1..9: note = linker amino acid sequence
GGGGSGGGG

SEQ ID NO: 72 - protein, 14 aa, synthetic construct
  REGION 1..14: note = linker amino acid sequence
GGGGSGGGGSGGGG

SEQ ID NO: 73 - protein, 15 aa, synthetic construct
  REGION 1..15: note = LILRB2-Fc linker
GGGGSGGGGSGGGGS

SEQ ID NO: 74 - protein, 20 aa, synthetic construct
  REGION 1..20: note = linker amino acid sequence
GGGGSGGGGSGGGGSGGGGS

SEQ ID NO: 75 - protein, 8 aa, synthetic construct
  REGION 1..8: note = linker amino acid sequence
GGGGGGGG

SEQ ID NO: 76 - protein, 8 aa, synthetic construct
  REGION 1..8: note = linker amino acid sequence
GGGGGGGG

SEQ ID NO: 77 - protein, 15 aa, synthetic construct
  REGION 1..15: note = linker amino acid sequence
  REPEAT 6..10: note = may be absent
  REPEAT 11..15: note = may be absent
EAAAKEAAAKEAAAK

SEQ ID NO: 78 - protein, 27 aa, synthetic construct
  REGION 1..27: note = linker amino acid sequence
  REPEAT 2..6
  REPEAT 7..11
  REPEAT 12..16: note = may be absent
  REPEAT 17..21: note = may be absent
  REPEAT 22..26: note = may be absent
AEAAAKEAAAKEAAAKEAAAKEAAAKA

SEQ ID NO: 79 - protein, 12 aa, synthetic construct
  REGION 1..12: note = linker amino acid sequence
AEAAAKEAAAKA

SEQ ID NO: 80 - protein, 46 aa, synthetic construct
  REGION 1..46: note = linker amino acid sequence
AEAAAKEAAAKEAAAKEAAAKALEAEAAAKEAAAKEAAAKEAAAKA

SEQ ID NO: 81 - protein, 5 aa, synthetic construct
  REGION 1..5: note = linker amino acid sequence
PAPAP

SEQ ID NO: 82 - protein, 18 aa, synthetic construct
  REGION 1..18: note = linker amino acid sequence
KESGSVSSEQLAQFRSLD

SEQ ID NO: 83 - protein, 14 aa, synthetic construct
  REGION 1..14: note = linker amino acid sequence
EGKSSGSGSESKST

SEQ ID NO: 84 - protein, 12 aa, synthetic construct
  REGION 1..12: note = linker amino acid sequence
GSAGSAAGSGEF

SEQ ID NO: 85 - protein, 232 aa, synthetic construct
  REGION 1..232: note = hIgG1 C220S Fc Hole plus S-S
EPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 86 - protein, 232 aa, synthetic construct
  REGION 1..232: note = hIgG1 C220S Fc knob plus S-S
EPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 87 - DNA, 45 bp, synthetic construct
  misc_feature 1..45: note = LILRB2-Fc linker (GGGGS)x3 NA of 73
  source 1..45: mol_type = other DNA
ggaggaggaggctccggaggaggaggctctggcggcggcggcagc

SEQ ID NO: 88 - protein, 117 aa, synthetic construct
  REGION 1..117: note = SIRP short (AA 31-147) (UniProt ID P78324)
EEELQVIQPDKSVLVAAGETATLRCTATSLIPVGPIQWFRGAGPGRELIYNQKEGHFPRVTTVSDLTKRNNMDFSIRIGNITPADAGTYYCVKFRKGSPDDVEFKSGAGTELSVRAK

SEQ ID NO: 89 - DNA, 351 bp, synthetic construct
  misc_feature 1..351: note = NA of 88 SIRP short (AA 31-147) (UniProt ID P78324)
  source 1..351: mol_type = other DNA
gaggaggagctgcaggtcatccagcccgataagtctgtgctggtggcagcaggagagaccgccacactgaggtgcaccgccacaagcctgatcccagtgggaccaatccagtggtttaggggagcaggccctggcagagagctgatctacaaccagaaggagggccacttcccaagagtgaccacagtgagcgacctgaccaagcggaacaatatggatttttccatcagaatcggcaatatcacacctgccgacgccggcacctactattgcgtgaagttcaggaagggctccccagacgatgtggagtttaagagcggagcaggcaccgagctgtccgtgcgggcaaag

SEQ ID NO: 90 - protein, 359 aa, synthetic construct
  REGION 1..359: note = SIRP short Fc-IgG1 knob LALA
EEELQVIQPDKSVLVAAGETATLRCTATSLIPVGPIQWFRGAGPGRELIYNQKEGHFPRVTTVSDLTKRNNMDFSIRIGNITPADAGTYYCVKFRKGSPDDVEFKSGAGTELSVRAKGGGGSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 91 - DNA, 1077 bp, synthetic construct
  misc_feature 1..1077: note = NA of 90 SIRP short Fc-IgG1 knob LALA
  source 1..1077: mol_type = other DNA
gaggaggagctgcaggtcatccagcccgataagtctgtgctggtggcagcaggagagaccgccacactgaggtgcaccgccacaagcctgatcccagtgggaccaatccagtggtttaggggagcaggccctggcagagagctgatctacaaccagaaggagggccacttcccaagagtgaccacagtgagcgacctgaccaagcggaacaatatggatttttccatcagaatcggcaatatcacacctgccgacgccggcacctactattgcgtgaagttcaggaagggctccccagacgatgtggagtttaagagcggagcaggcaccgagctgtccgtgcgggcaaagggaggaggaggcagcggaggaggaggctccgagcctaagagctccgacaagacccacacatgcccaccatgtcctgcaccagaggcagcaggaggaccttccgtgttcctgtttcctccaaagccaaaggatacactgatgatctccagaacaccagaggtgacctgcgtggtggtggacgtgtctcacgaggaccccgaggtgaagtttaactggtacgtggacggcgtggaggtgcacaatgccaagaccaagccaagggaggagcagtacaactccacatatcgcgtggtgtctgtgctgaccgtgctgcaccaggattggctgaacggcaaggagtataagtgtaaggtgagcaataaggccctgcccgcccctatcgagaagaccatctccaaggcaaagggacagcccagggagcctcaggtgtacacactgcccccttgccgcgacgagctgaccaagaaccaggtgtctctgtggtgtctggtgaagggcttctacccatctgacatcgccgtggagtgggagagcaatggccagcccgagaacaattacaagaccacaccacccgtgctggacagcgatggctccttctttctgtattccaagctgacagtggacaagtctcggtggcagcagggcaacgtgttttcctgttctgtgatgcacgaggccctgcacaatcactatacccagaagagcctgtccctgtctcccggcaag

SEQ ID NO: 92 - DNA, 696 bp, synthetic construct
  misc_feature 1..696: note = NA of 85 hIgG1 C220S Fc NO LALA Hole
  source 1..696: mol_type = other DNA
gagcctaagagctccgacaagacccacacatgcccaccttgtccagcacctgagctgctgggaggaccatccgtgttcctgtttccacccaagcctaaggataccctgatgatctctcgcacccctgaggtgacatgcgtggtggtggacgtgagccacgaggaccccgaggtgaagtttaactggtacgtggacggcgtggaggtgcacaatgccaagacaaagccccgggaggagcagtacaacagcacctatagagtggtgtccgtgctgacagtgctgcaccaggattggctgaacggcaaggagtacaagtgtaaggtgtccaataaggccctgccagcccccatcgagaagaccatctctaaggcaaagggacagcccagggagcctcaggtgtgcaccctgcctccaagccgcgacgagctgacaaagaaccaggtgtctctgagctgtgccgtgaagggcttctacccatctgacatcgccgtggagtgggagagcaatggccagcccgagaacaattataagaccacaccccctgtgctggactctgatggcagcttctttctggtgtccaagctgaccgtggataagtctaggtggcagcagggcaacgtgttttcctgttctgtgatgcacgaggccctgcacaatcactacacacagaagagcctgtccctgtctcccggcaag

SEQ ID NO: 93 - DNA, 695 bp, synthetic construct
  misc_feature 1..695: note = NA of 86 hIgG1 C220S Fc NO LALA knob
  source 1..695: mol_type = other DNA
gagcctaagagctccgacaagacccacacatgcccaccatgtcctgcaccagagctgctgggaggaccttccgtgttcctgtttcctccaaagccaaaggatacactgatgatctccagaacaccagaggtgacctgcgtggtggtggacgtgtctcacgaggaccccgaggtgaagtttaactggtacgtggacggcgtggaggtgcacaatgccaagaccaagccaagggaggagcagtacaactccacatatcgcgtggtgtctgtgctgaccgtgctgcaccaggattggctgaacggcaaggagtataagtgtaaggtgagcaataaggccctgcccgcccctatcgagaagaccatctccaaggcaaagggacagcccagggagcctcaggtgtacacactgcccccttgccgcgacgagctgaccaagaaccaggtgtctctgtggtgtctggtgaagggcttctacccatctgacatcgccgtggagtgggagagcaatggccagcccgagaacaattacaagaccacaccacccgtgctggacagcgatggctccttctttctgtattccaagctgacagtggacaagtctcggtggcagcagggcaacgtgttttcctgttctgtgatgcacgaggccctgcacaatcactatacccagaagagcctgtccctgtctcccggcaa

SEQ ID NO: 94 - protein, 585 aa, synthetic construct
  REGION 1..585: note = SIRP linker-IgG1 NO LALA knob Fc
EEELQVIQPDKSVLVAAGETATLRCTATSLIPVGPIQWFRGAGPGRELIYNQKEGHFPRVTTVSDLTKRNNMDFSIRIGNITPADAGTYYCVKFRKGSPDDVEFKSGAGTELSVRAKPSAPVVSGPAARATPQHTVSFTCESHGFSPRDITLKWFKNGNELSDFQTNVDPVGESVSYSIHSTAKVVLTREDVHSQVICEVAHVTLQGDPLRGTANLSETIRVPPTLEVTQQPVRAENQVNVTCQVRKFYPQRLQLTWLENGNVSRTETASTVTENKDGTYNWMSWLLVNVSAHRDDVKLTCQVEHDGQPAVSKSHDLKVSAHPKEQGSNTAAENTGSNERNIYGGGGSGGGGSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 95 - DNA, 1755 bp, synthetic construct
  misc_feature 1..1755: note = NA of 94 SIRP linker-IgG1 NO LALA knob Fc
  source 1..1755: mol_type = other DNA
gaggaggagctgcaggtcatccagcccgataagtctgtgctggtggcagcaggagagaccgccacactgaggtgcaccgccacaagcctgatcccagtgggaccaatccagtggtttaggggagcaggccctggcagagagctgatctacaaccagaaggagggccacttcccaagagtgaccacagtgagcgacctgaccaagcggaacaatatggatttttccatcagaatcggcaatatcacacctgccgacgccggcacctactattgcgtgaagttcaggaagggctccccagacgatgtggagtttaagagcggagcaggcaccgagctgtccgtgcgggcaaagccttccgccccagtggtgtctggaccagcagccagagccaccccacagcacacagtgtccttcacctgtgagtctcacggctttagcccccgggacatcaccctgaagtggttcaagaacggcaatgagctgtctgactttcagaccaacgtggaccccgtgggcgagtctgtgagctattccatccactctacagccaaggtggtgctgacccgcgaggacgtgcacagccaggtcatctgcgaggtggcacacgtgaccctgcagggcgatcctctgaggggcacagccaatctgagcgagaccatcagagtgccccctacactggaggtgacccagcagcccgtgcgcgcagagaaccaagtgaatgtgacatgtcaggtgaggaagttctaccctcagcgcctgcagctgacctggctggagaacggcaacgtgagccggaccgagacagccagcaccgtgacagagaacaaggacggcacatataattggatgtcttggctgctggtgaacgtgagcgcccacagggacgatgtgaagctgacctgccaggtggagcacgacggacagccagccgtgtctaagagccacgatctgaaggtgagcgcccaccctaaggagcagggctccaacacagccgccgagaataccggcagcaacgagcggaatatctacggaggaggaggcagcggaggaggaggctccgagcctaagagctccgacaagacccacacatgcccaccatgtcctgcaccagagctgctgggaggaccttccgtgttcctgtttcctccaaagccaaaggatacactgatgatctccagaacaccagaggtgacctgcgtggtggtggacgtgtctcacgaggaccccgaggtgaagtttaactggtacgtggacggcgtggaggtgcacaatgccaagaccaagccaagggaggagcagtacaactccacatatcgcgtggtgtctgtgctgaccgtgctgcaccaggattggctgaacggcaaggagtataagtgtaaggtgagcaataaggccctgcccgcccctatcgagaagaccatctccaaggcaaagggacagcccagggagcctcaggtgtacacactgcccccttgccgcgacgagctgaccaagaaccaggtgtctctgtggtgtctggtgaagggcttctacccatctgacatcgccgtggagtgggagagcaatggccagcccgagaacaattacaagaccacaccacccgtgctggacagcgatggctccttctttctgtattccaagctgacagtggacaagtctcggtggcagcagggcaacgtgttttcctgttctgtgatgcacgaggccctgcacaatcactatacccagaagagcctgtccctgtctcccggcaag

SEQ ID NO: 96 - protein, 645 aa, synthetic construct
  REGION 1..645: note = LILRB2 - V57R -linker-IgG1 NO LALA hole Fc
QTGTIPKPTLWAEPDSVITQGSPVTLSCQGSLEAQEYRLYREKKSASWITRIRPELRKNGQFHIPSITWEHTGRYGCQYYSRARWSELSDPLVLVMTGAYPKPTLSAQPSPVVTSGGRVTLQCESQVAFGGFILCKEGEEEHPQCLNSQPHARGSSRAIFSVGPVSPNRRWSHRCYGYDLNSPYVWSSPSDLLELLVPGVSKKPSLSVQPGPVVAPGESLTLQCVSDVGYDRFVLYKEGERDLRQLPGRQPQAGLSQANFTLGPVSRSYGGQYRCYGAHNLSSECSAPSDPLDILITGQIRGTPFISVQPGPTVASGENVTLLCQSWRQFHTFLLTKAGAADAPLRLRSIHEYPKYQAEFPMSPVTSAHAGTYRCYGSLNSDPYLLSHPSEPLELVVSGGGGSGGGGSGGGGSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 97 - DNA, 1935 bp, synthetic construct
  misc_feature 1..1935: note = 97 NA of 96 LILRB2 - V57R -linker-IgG1 NO LALA hole Fc
  source 1..1935: mol_type = other DNA
cagaccggcacaatcccaaagcccaccctgtgggccgagcctgattccgtgatcacccagggctctccagtgacactgtcctgccagggctctctggaggcccaggagtaccggctgtatagagagaagaagtctgccagctggatcacccggatcagacctgagctgagaaagaacggccagtttcacatcccaagcatcacctgggagcacacaggccggtacggatgccagtactattcccgggccagatggagcgagctgtccgaccctctggtgctggtcatgaccggcgcctatcctaagccaacactgagcgcccagccatcccctgtggtgaccagcggcggcagagtgacactgcagtgtgagtcccaggtggccttcggcggctttatcctgtgcaaggagggcgaggaggagcacccacagtgtctgaacagccagccacacgcccggggcagctccagagccatcttctccgtgggacccgtgagcccaaaccggagatggagccaccggtgctacggctatgacctgaatagcccttacgtgtggtctagcccatccgatctgctggagctgctggtgcccggcgtgtccaagaagccttccctgtctgtgcagccaggaccagtggtggcaccaggagagtctctgaccctgcagtgcgtgagcgacgtgggctacgatcggttcgtgctgtataaggagggagagagggatctgaggcagctgccaggcagacagccacaggccggcctgagccaggccaactttacactgggcccagtgagcaggtcctatggcggacagtacaggtgctatggagcacacaatctgtcctctgagtgttctgcccccagcgaccccctggacatcctgatcaccggccagatcaggggcacacccttcatctccgtgcagcctggaccaaccgtggcctctggcgagaacgtgacactgctgtgccagtcttggcgccagttccacacctttctgctgacaaaggcaggagcagcagacgcaccactgaggctgcgcagcatccacgagtaccccaagtatcaggccgagtttccaatgtctccagtgaccagcgcccacgcaggcacatacaggtgttatggcagcctgaacagcgacccctacctgctgagccacccttccgagccactggagctggtggtgagcggaggaggaggctccggaggaggaggctctggcggcggcggcagcgagcctaagagctccgacaagacccacacatgcccaccttgtccagcacctgagctgctgggaggaccatccgtgttcctgtttccacccaagcctaaggataccctgatgatctctcgcacccctgaggtgacatgcgtggtggtggacgtgagccacgaggaccccgaggtgaagtttaactggtacgtggacggcgtggaggtgcacaatgccaagacaaagccccgggaggagcagtacaacagcacctatagagtggtgtccgtgctgacagtgctgcaccaggattggctgaacggcaaggagtacaagtgtaaggtgtccaataaggccctgccagcccccatcgagaagaccatctctaaggcaaagggacagcccagggagcctcaggtgtgcaccctgcctccaagccgcgacgagctgacaaagaaccaggtgtctctgagctgtgccgtgaagggcttctacccatctgacatcgccgtggagtgggagagcaatggccagcccgagaacaattataagaccacaccccctgtgctggactctgatggcagcttctttctggtgtccaagctgaccgtggataagtctaggtggcagcagggcaacgtgttttcctgttctgtgatgcacgaggccctgcacaatcactacacacagaagagcctgtccctgtctcccggcaag

SEQ ID NO: 98 - protein, 359 aa, synthetic construct
  REGION 1..359: note = SIRP short linker-IgG1 NO LALA knob Fc
EEELQVIQPDKSVLVAAGETATLRCTATSLIPVGPIQWFRGAGPGRELIYNQKEGHFPRVTTVSDLTKRNNMDFSIRIGNITPADAGTYYCVKFRKGSPDDVEFKSGAGTELSVRAKGGGGSGGGGSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 99 - DNA, 1077 bp, synthetic construct
  misc_feature 1..1077: note = NA of 98 SIRP short linker-IgG1 NO LALA knob Fc
  source 1..1077: mol_type = other DNA
gaggaggagctgcaggtcatccagcccgataagtctgtgctggtggcagcaggagagaccgccacactgaggtgcaccgccacaagcctgatcccagtgggaccaatccagtggtttaggggagcaggccctggcagagagctgatctacaaccagaaggagggccacttcccaagagtgaccacagtgagcgacctgaccaagcggaacaatatggatttttccatcagaatcggcaatatcacacctgccgacgccggcacctactattgcgtgaagttcaggaagggctccccagacgatgtggagtttaagagcggagcaggcaccgagctgtccgtgcgggcaaagggaggaggaggcagcggaggaggaggctccgagcctaagagctccgacaagacccacacatgcccaccatgtcctgcaccagagctgctgggaggaccttccgtgttcctgtttcctccaaagccaaaggatacactgatgatctccagaacaccagaggtgacctgcgtggtggtggacgtgtctcacgaggaccccgaggtgaagtttaactggtacgtggacggcgtggaggtgcacaatgccaagaccaagccaagggaggagcagtacaactccacatatcgcgtggtgtctgtgctgaccgtgctgcaccaggattggctgaacggcaaggagtataagtgtaaggtgagcaataaggccctgcccgcccctatcgagaagaccatctccaaggcaaagggacagcccagggagcctcaggtgtacacactgcccccttgccgcgacgagctgaccaagaaccaggtgtctctgtggtgtctggtgaagggcttctacccatctgacatcgccgtggagtgggagagcaatggccagcccgagaacaattacaagaccacaccacccgtgctggacagcgatggctccttctttctgtattccaagctgacagtggacaagtctcggtggcagcagggcaacgtgttttcctgttctgtgatgcacgaggccctgcacaatcactatacccagaagagcctgtccctgtctcccggcaag

SEQ ID NO: 100 - protein, 577 aa, synthetic construct
  REGION 1..577: note = LILRB2 ECD Q8N423-2 D161 M235
QTGTIPKPTLWAEPDSVITQGSPVTLSCQGSLEAQEYRLYREKKSASWITRIRPELVKNGQFHIPSITWEHTGRYGCQYYSRARWSELSDPLVLVMTGAYPKPTLSAQPSPVVTSGGRVTLQCESQVAFGGFILCKEGEDEHPQCLNSQPHARGSSRAIFSVGPVSPNRRWSHRCYGYDLNSPYVWSSPSDLLELLVPGVSKKPSLSVQPGPVMAPGESLTLQCVSDVGYDRFVLYKEGERDLRQLPGRQPQAGLSQANFTLGPVSRSYGGQYRCYGAHNLSSECSAPSDPLDILITGQIRGTPFISVQPGPTVASGENVTLLCQSWRQFHTFLLTKAGAADAPLRLRSIHEYPKYQAEFPMSPVTSAHAGTYRCYGSLNSDPYLLSHPSEPLELVVSGPSMGSSPPPTGPISTPAGPEDQPLTPTGSDPQSGLGRHLGVVIGILVAVVLLLLLLLLLFLILRHRRQGKHWTSTQRKADFQHPAGAVGPEPTDRGLQWRSSPAADAQEENLYAAVKDTQPEDGVEMDTRAAASEAPQDVTYAQLHSLTLRRKATEPPPSQEREPPAEPSIYATLAIH

SEQ ID NO: 101 - protein, 645 aa, synthetic construct
  REGION 1..645: note = LILRB2 -linker-IgG1 Fc NO LALA hole
QTGTIPKPTLWAEPDSVITQGSPVTLSCQGSLEAQEYRLYREKKSASWITRIRPELVKNGQFHIPSITWEHTGRYGCQYYSRARWSELSDPLVLVMTGAYPKPTLSAQPSPVVTSGGRVTLQCESQVAFGGFILCKEGEEEHPQCLNSQPHARGSSRAIFSVGPVSPNRRWSHRCYGYDLNSPYVWSSPSDLLELLVPGVSKKPSLSVQPGPVVAPGESLTLQCVSDVGYDRFVLYKEGERDLRQLPGRQPQAGLSQANFTLGPVSRSYGGQYRCYGAHNLSSECSAPSDPLDILITGQIRGTPFISVQPGPTVASGENVTLLCQSWRQFHTFLLTKAGAADAPLRLRSIHEYPKYQAEFPMSPVTSAHAGTYRCYGSLNSDPYLLSHPSEPLELVVSGGGGSGGGGSGGGGSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 102 - protein, 438 aa, synthetic construct
  REGION 1..438: note = LILRB1 ECD (AA 24-461)
GHLPKPTLWAEPGSVITQGSPVTLRCQGGQETQEYRLYREKKTALWITRIPQELVKKGQFPIPSITWEHAGRYRCYYGSDTAGRSESSDPLELVVTGAYIKPTLSAQPSPVVNSGGNVILQCDSQVAFDGFSLCKEGEDEHPQCLNSQPHARGSSRAIFSVGPVSPSRRWWYRCYAYDSNSPYEWSLPSDLLELLVLGVSKKPSLSVQPGPIVAPEETLTLQCGSDAGYNRFVLYKDGERDFLQLAGAQPQAGLSQANFTLGPVSRSYGGQYRCYGAHNLSSEWSAPSDPLDILIAGQFYDRVSLSVQPGPTVASGENVTLLCQSQGWMQTFLLTKEGAADDPWRLRSTYQSQKYQAEFPMGPVTSAHAGTYRCYGSQSSKPYLLTHPSDPLELVVSGPSGGPSSPTTGPTSTSGPEDQPLTPTGSDPQSGLGRHLGV

SEQ ID NO: 103 - protein, 438 aa, synthetic construct
  REGION 1..438: note = LILRB1 ECD (AA 24-461) plus V12 analog (V55 to R)
GHLPKPTLWAEPGSVITQGSPVTLRCQGGQETQEYRLYREKKTALWITRIPQELRKKGQFPIPSITWEHAGRYRCYYGSDTAGRSESSDPLELVVTGAYIKPTLSAQPSPVVNSGGNVILQCDSQVAFDGFSLCKEGEDEHPQCLNSQPHARGSSRAIFSVGPVSPSRRWWYRCYAYDSNSPYEWSLPSDLLELLVLGVSKKPSLSVQPGPIVAPEETLTLQCGSDAGYNRFVLYKDGERDFLQLAGAQPQAGLSQANFTLGPVSRSYGGQYRCYGAHNLSSEWSAPSDPLDILIAGQFYDRVSLSVQPGPTVASGENVTLLCQSQGWMQTFLLTKEGAADDPWRLRSTYQSQKYQAEFPMGPVTSAHAGTYRCYGSQSSKPYLLTHPSDPLELVVSGPSGGPSSPTTGPTSTSGPEDQPLTPTGSDPQSGLGRHLGV

SEQ ID NO: 104 - protein, 84 aa, synthetic construct
  REGION 1..84: note = D1 domain of LILRB2
PKPTLWAEPDSVITQGSPVTLSCQGSLEAQEYRLYREKKSASWITRIRPELVKNGQFHIPSITWEHTGRYGCQYYSRARWSELS

SEQ ID NO: 105 - protein, 89 aa, synthetic construct
  REGION 1..89: note = D1 domain of LILRB1
PKPTLWAEPGSVITQGSPVTLRCQGGQETQEYRLYREKKTALWITRIPQELVKKGQFPIPSITWEHAGRYRCYYGSDTAGRSESSDPLE

SEQ ID NO: 106 - DNA, 1935 bp, synthetic construct
  misc_feature 1..1935: note = NA of 101 LILRB2-linker-IgG1 Fc NO LALA hole
  source 1..1935: mol_type = other DNA
cagaccggcacaatcccaaagcccaccctgtgggccgagcctgattccgtgatcacccagggctctccagtgacactgtcctgccagggctctctggaggcccaggagtaccggctgtatagagagaagaagtctgccagctggatcacccggatcagacctgagctggtgaagaacggccagtttcacatcccaagcatcacctgggagcacacaggccggtacggatgccagtactattcccgggccagatggagcgagctgtccgaccctctggtgctggtcatgaccggcgcctatcctaagccaacactgagcgcccagccatcccctgtggtgaccagcggcggcagagtgacactgcagtgtgagtcccaggtggccttcggcggctttatcctgtgcaaggagggcgaggaggagcacccacagtgtctgaacagccagccacacgcccggggcagctccagagccatcttctccgtgggacccgtgagcccaaaccggagatggagccaccggtgctacggctatgacctgaatagcccttacgtgtggtctagcccatccgatctgctggagctgctggtgcccggcgtgtccaagaagccttccctgtctgtgcagccaggaccagtggtggcaccaggagagtctctgaccctgcagtgcgtgagcgacgtgggctacgatcggttcgtgctgtataaggagggagagagggatctgaggcagctgccaggcagacagccacaggccggcctgagccaggccaactttacactgggcccagtgagcaggtcctatggcggacagtacaggtgctatggagcacacaatctgtcctctgagtgttctgcccccagcgaccccctggacatcctgatcaccggccagatcaggggcacacccttcatctccgtgcagcctggaccaaccgtggcctctggcgagaacgtgacactgctgtgccagtcttggcgccagttccacacctttctgctgacaaaggcaggagcagcagacgcaccactgaggctgcgcagcatccacgagtaccccaagtatcaggccgagtttccaatgtctccagtgaccagcgcccacgcaggcacatacaggtgttatggcagcctgaacagcgacccctacctgctgagccacccttccgagccactggagctggtggtgagcggaggaggaggctccggaggaggaggctctggcggcggcggcagcgagcctaagagctccgacaagacccacacatgcccaccttgtccagcacctgagctgctgggaggaccatccgtgttcctgtttccacccaagcctaaggataccctgatgatctctcgcacccctgaggtgacatgcgtggtggtggacgtgagccacgaggaccccgaggtgaagtttaactggtacgtggacggcgtggaggtgcacaatgccaagacaaagccccgggaggagcagtacaacagcacctatagagtggtgtccgtgctgacagtgctgcaccaggattggctgaacggcaaggagtacaagtgtaaggtgtccaataaggccctgccagcccccatcgagaagaccatctctaaggcaaagggacagcccagggagcctcaggtgtgcaccctgcctccaagccgcgacgagctgacaaagaaccaggtgtctctgagctgtgccgtgaagggcttctacccatctgacatcgccgtggagtgggagagcaatggccagcccgagaacaattataagaccacaccccctgtgctggactctgatggcagcttctttctggtgtccaagctgaccgtggataagtctaggtggcagcagggcaacgtgttttcctgttctgtgatgcacgaggccctgcacaatcactacacacagaagagcctgtccctgtctcccggcaag